(19) United States
(12) Patent Application Publication
(10) Pub. No.: US 2004/0092524 A1
(43) Pub. Date: May 13, 2004

(54) Novel aminophenyl piperazine or aminophenyl piperidine derivatives inhibiting prenyl transferase proteins and method for preparing same

(76) Inventors: Michel Perez, Castres (FR); Marie Lamothe, Castres (FR); Anna Kruczynski, Castres (FR); Bridget Hill, Castres (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/399,069

(22) PCT Filed: Oct. 10, 2001
(86) PCT No.: PCT/FR01/03121

(30) Foreign Application Priority Data
Oct. 10, 2000 (FR) 00/12919

(51) Int. Cl. A61K 31/496; A61K 31/517; C07D 417/02; C07D 413/02; C 07D 4 3/02

(52) U.S. Cl. 514/249; 514/252.17; 514/253.01; 514/254.02; 514/254.03; 514/254.05; 514/254.06; 514/266.22; 514/326; 544/284; 544/353; 544/360; 544/368; 544/369; 544/370

(57) ABSTRACT

The invention concerns compounds corresponding to general formula (I), wherein, in particular: W represents hydrogen, COR₆, CSR₆, SO₂R₆, CO(CH₂)ₙR₆, (CH₂)ₙR₇; X represents CH or N; Y represents (CH₂)ₙ, CO, CH₂CO, CH═CHCO, CH₂CH₂CO; Z represents a heterocycle. When Z=pyridine, then Y is other than CO. R₁ represents hydrogen, C₁-C₆ alkyl, halogen OCH₃, CF₃; R₂ and R₃, identical or different, represent hydrogen, C₁-C₆ alkyl; R₄ represents a) hydrogen, b) C₁-C₆ alkyl, c) an aryl, d) a heterocycle; R₅ represents hydrogen, COR₇R₈, SO₂R₇, CO(CH₂)ₙSR₇, CO(CH₂)ₙOR₇, CONR₇R₈, CO(CH₂)ₘCOR₇; R₆ represents a) a phenyl or a naphthyl, b) a C₁-C₆ alkyl, a cycloalkyl, c) a heterocycle, d) NR₇R₈; R₇ and R₈, identical or different, represent a) hydrogen, C₁-C₁₅ alkyl, b) a heterocycle, c) an aryl; n represents 0 to 10; m represents 2 to 10; provided that when Z represents a quinozaline or benzimidazole group, then R₅ is other than CH₂Ph or methyl and n is other than zero.

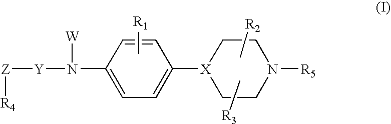

The present invention relates to novel aminophenyl piperazine or aminophenyl piperidine derivatives, to a process for manufacturing them, to pharmaceutical compositions containing them and to their use as medicinal products.

The ras oncogenes (Ha-ras, Ki4a-ras, Ki4b-ras and N-ras) are present in many human cancers, for instance cancer of the pancreas and of the colon, and also in certain types of leukemia (Barbacid M. Ann. Rev. Biochem., 1987, 56:779-827; Bos J.-L. Cancer Res., 1989, 49: 46824689). The Ras proteins are involved in the signaling process that links the growth factors, of the cell surface, to cell proliferation.

In normal cells, biochemical studies have shown that the Ras proteins in inactive form are linked to GDP. After activation of the growth factor receptors, the Ras proteins exchange the GDP for GTP and undergo a conformational change. This activated form of the Ras protein propagates the growth signal until the Ras protein returns to its inactive form by hydrolysis of the GTP to GDP. Mutated Ras proteins, derived from the ras oncogenes, remain in the activated form and as a result transmit a permanent growth signal (Polakis P. and McCormick F. J. Biol. Chem, 1993, 268:13, 9157-9160; Glomset J. A. and Farnsworth C C. Annu. Rev. Cell. Biol., 1994, 10:181-205).

In all cases, the Ras proteins must be associated with the cell membrane in order to be active. This process especially involves the addition of an isoprenoid unit (C15 or C20) to the cysteine of terminal tetrapeptide of the Ras proteins known as the “CAAX box” (in which C represents a cysteine, A an aliphatic amino acid, and X any amino acid).

This alkylation is catalyzed, depending on the nature of the sequence, by the enzyme Protein Farnesyl Transferase (PFTase) or by the enzyme Protein Geranyl Geranyl Transferase (PGGTase I) which respectively transfer a farnesyl (C15) or geranyl geranyl (C20) group.

Blockage of the function of the Ras proteins should result in inhibition of the growth of the tumoral cells which depend on the activation of Ras or which express mutated Ras proteins (Perrin D., Halazy S. and Hill B. T. J. Enzyme Inhi., 1996; 11:77-95; Levy R. Presse Med., 1995, 24:725-729 ; Sebolt-Leopold J. S. Emerging Drugs, 1996, 1:219-239 ; Hamilton A. D. and Sebti S. M. Drugs News Perspect, 1995, 8:138-145; Der C. J., Cox A. D., Sebti S. M. and Hamilton A. D. Anti-CancerDrugs, 1996, 7:165-172 ; Halazy S., Gotteland J.-P., Lamothe M., Perrin D. and Hill B. T. Drugs of the Future, 1997, 22:1133-1146 ; Rowinsky E. K., Windle J. J, Von Hoff D. D. J. Clin. Oncol., 1999, 17:3631-3652).

The inhibition of PFIase and/or of PGGTase I and thus of the prenylation of the Ras proteins makes it possible to control the proliferation of the ras-mutated cancer cells. This has been demonstrated using PFTase inhibitors such as BZA-5B (James G. L., Goldstein J.-L., Brown M. S. et al Science, 1993, 260:1937-1942) or L-731,734 (Kohl N. E., Mosser S. D., De Solms S. J. et al. Science, 1993, 260:1934-1937) on cell proliferation, and also with ras-dependent grafted tumors in mice (Kohl N. E., Wilson F. R., Mosser S. D. et al. Proc. Natl. Acad. Sci. USA, 1994, 91:9141-9145; Kohl N. E., Omer C. A., Conner M. W. et al. Nature Med., 1995, 1:792-797). This has also been demonstrated using PGGTase I inhibitors on cell differentiation and proliferation (Lerner E. C. Hamilton A. D. and Sebti S. M. Anti-Cancer Drug Design, 1997, 12:229-238 ; Sun J. et al Cancer Research, 1999, 59:4919-4926). PFTase and/or PGGTase I inhibitors may thus be useful as anticancer agents since they can serve to control cell proliferation in tumors in which the farnesylation of proteins plays a determining role. These inhibitors may also be useful in controlling the proliferation of smooth muscle cells (Indolfi et al. Nature Med, 1995, 1:541-545) and are therefore potentially useful for treating or preventing atherosclerosis and restenosis (JP H7-112930).

One subject of the present invention is a novel class of protein prenylation inhibitors and more particularly of PFTase and PGGTase I inhibitors, which are distinguished from the prior art by their different chemical structure and their noteworthy biological property.

A subject of the present invention is piperazines or piperidines derived from anilines, which have the capacity of inhibiting PFTase or PGGTase I not only at the enzymatic level but also at the cellular level.

The prior art in this field is illustrated especially by:

tricyclic compounds possibly containing a piperazine or a piperidine and described as PFTase inhibitors (WO 96/31477, WO 95/10514, WO 95/10515, WO 95/10516, WO 97/23478)

carbonyl-piperazinyl or carbonyl-piperidine compounds described as PFTase and squalene synthase inhibitors (WO 96/31501).

The compounds of the present invention have the general formula (I):

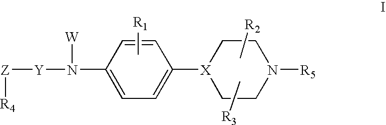

in which

W represents: hydrogen, COR₆, CSR₆, SO₂R₆, CO(CH₂)ₙR₆, (CH₂)ₙR₇

X represents: CH or N

Y represents: (CH₂)ₙ, CO, CH₂CO, CH═CHCO, CH₂CH₂CO.

when Y=CO, CH₂CO, CH═CH—CO or CH₂CH₂—CO then W represents only hydrogen,

Z represents: imidazole, benzimidazole, isoxazole, tetrazole, oxadiazole, thiazole, thiadiazole, pyridine, quinazoline, quinoxaline. These heterocycles may be unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH, CONHOH, SO₂NH₂, CONH₂.

When Z=pyridine, then Y is other than CO

R₁ represents: hydrogen, C₁-C₆ alkyl, halogen, OCH₃, CF₃

R₂ and R₃, which may be identical or different, represent: hydrogen, C₁-C₆ alkyl

R₄ represents:

a) hydrogen,

b) C₁-C₆ alkyl which is unsubstituted or substituted with an aryl, a cyanophenyl, a nitrophenyl, an aminophenyl, a methoxyphenyl, a hydroxyphenyl, a heterocycle, a halogen, CN, NO₂, OR₂, SR₂, NR₂R₃ COOR₂;

c) an aryl,

d) a heterocycle.

R₅ represents: hydrogen, COR₇, SO₂R₇, CO(CH₂)ₙSR₇, CO(CH₂)ₙOR₇, CONR₇R₈, CSNR₇R₈, CO(CH₂)ₘCOR₇

R₆ represents:

a) a phenyl or a naphthyl which is unsubstituted or substituted with one or more residues chosen from C₁-C₆ alkyl, halogen, phenyl, naphthyl, NO₂, CN, CF₃, OR₇, SR₇, NR₇R₈, COOR₇, CONR₇R₈, COR₇,

b) a C₁-C₆ alkyl, a cycloalkyl,

c) a heterocycle,

d) NR₇R₈

R₇ and R₈, which may be identical or different, represent:

a) hydrogen; C₁-C₁₅ alkyl, which is unsubstituted or substituted with a halogen, COOMe, COOH, OMe, OH, CF₃, CN, SMe; a cycloalkyl which is unsubstituted or substituted with a halogen, OMe, OH, CF₃, CN, SMe; an alkylcycloalkyl which is unsubstituted or substituted with a halogen, OMe, OH, CF₃, CN, SMe; or R₇ and R₈ when they are adjacent, taken together, can form a 4- to 6-membered ring with the nitrogen atom to which they are attached,

b) a heterocycle, an alkylheterocycle,

c) an aryl, an alkylaryl, n represents: 0 to 10 m represents: 2 to 10 and also the therapeutically acceptable salts and solvates thereof.

In the preceding definitions:

all the combinations of substituents or of variables are possible provided that they lead to stable compounds.

The term “alkyl” represents linear or branched, saturated or unsaturated aliphatic hydrocarbon-based chains, unsubstituted or substituted with an NH₂, OH or phenyl and containing the specified number of carbon atoms.

The term “cycloalkyl” represents cyclic hydrocarbon-based chains containing from 3 to 10 carbon atoms.

The term “halogen” represents a fluorine, chlorine, bromine or iodione.

The term “aryl” represents any monocyclic or bicyclic carbon-based ring possibly containing up to 7 atoms per ring and in which at least one of the rings is aromatic. Examples that may be mentioned include a phenyl, biphenyl, naphthyl, tetrahydronaphthyl or indanyl. These aromatic nuclei may be unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH.

The term “heterocycle” represents either a stable monocycle containing from 5 to 7 atoms or a stable bicycle containing from 8 to 11 atoms, which may be either saturated or unsaturated, and may consist of carbon atoms and of one to four hetero atoms chosen from N, O and S. Monocyclic heterocycles fused to a benzene nucleus are also included in the definition of bicycles. Examples that may be mentioned include a residue chosen from furan, pyrrole, thiophene, thiazole, isothiazole, oxadiazole, imidazole, oxazole, isoxazole, pyridine, pyrimidine, quinazoline, quinoline, quinoxaline, tetrahydroquinoline, benzofurane, benzothiophene, indole, indoline, benzothiazole, benzothienyl, benzopyran, benzoxazole, benzo[1,3]dioxole, benzisoxazole, benzimidazole, chroman, dihydrobenzofuran, dihydrobenzothienyl, isoquinoline, morpholine, piperazine and piperidine. These heterocycles may be unsubstituted or substituted with one or more groups chosen from C₁-C₁₅ alkyl, halogen, OMe, CN, NO₂, OH, CF₃, OCF₃, OCH₂Ph, SMe, COOMe, COOEt and COOH.

In the terms “alkylcycloalkyl”, “alkylaryl” and “alkylheterocycle”, the prefix “alkyl” represents linear or branched, saturated or unsaturated aliphatic hydrocarbon-based chains comprising the specified number of carbon atoms and preceding the groups mentioned, the definition of which has been given previously.

The therapeutically acceptable salts of the compounds of the present invention comprise the conventional nontoxic salts of the compounds of the invention, such as those formed from organic or mineral acids. Examples that may be mentioned include the salts derived from mineral acids, for instance hydrochloric acid, hydrobromic acid, phosphoric acid or sulfuric acid, and those derived from organic acids, for instance acetic acid, trifluoroacetic acid, propionic acid, succinic acid, fumaric acid, malic acid, tartaric acid, citric acid, ascorbic acid, maleic acid, glutamic acid, benzoic acid, salicylic acid, toluenesulfonic acid, methanesulfonic acid, stearic acid or lactic acid.

These salts may be synthesized from the compounds according to the invention containing a basic portion and the corresponding acids according to the conventional chemical methods.

The therapeutically acceptable solvates of the compounds of the present invention comprise conventional solvates such as those formed during the final step of preparation of the compounds of the invention due to the presence of solvents. Examples that may be mentioned include the solvates due to the presence of water or ethanol.

All the stereoisomers, including all the optical isomers, of the compounds of general formula (I) also form part of the present invention, as does the mixture thereof in racemic form.

Among the compounds of general formula (I) forming part of the present invention, one category of compounds that is particularly suitable corresponds to the compounds of general formula (I) in which R₁, R₂, R₃ and R₄ each represent a hydrogen and Y represents a methylene (CH₂).

Another category of compounds forming part of the present invention that is particularly satisfactory corresponds to the compounds of general formula (I) in which Z represents an imidazolyl or pyridyl residue.

A third category of compounds forming part of the present invention that is particularly satisfactory corresponds to the compounds of general formula (I) in which Z represents an imidazolyl residue and R₄ represents a benzyl group substituted with a nitrile, nitro or methoxy group in position 4.

A fourth category of compounds forming part of the present invention that is particularly satisfactory corresponds to the compounds of general formula (I) in which X represents a nitrogen atom.

The present invention is also directed toward the category of compounds of general formula (I) in which R₅ represents a 2-thiophenecarbonyl, 5-chloro-2-thiophenecarbonyl or CSNR₇R₈ group.

The present invention also relates to the preparation of the compounds of general formula (I) by the general processes described in the synthetic schemes below, completed, where appropriate, by any standard manipulation described in the literature or well known to those skilled in the art, or else given as an example in the experimental section.

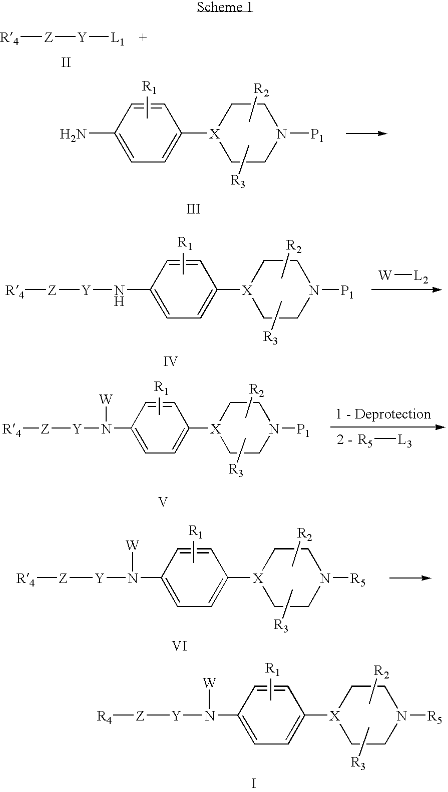

Scheme 1 illustrates the first general process that may be used for preparing the compounds of general formula (I). In the above general formulae, Z, Y, X, R₁, R₂, R₃, R₄, W and R₅ are defined as in the description preceding the general formula (I). R′₄ corresponds either to R₄ (defined above) or to a precursor of R₄, or to a protecting group of Z, or alternatively to a resin in the case of a synthesis on a solid support. This group R′₄ may be removed or converted at the end of the synthesis to allow the introduction of R₄. P₁ represents a protecting group. L₁ represents either a leaving group such as, for example, Cl, Br, I, OSO₂CH₃, OSO₂CF₃ or O-tosyl. In this case, the reaction with the amine of general formula (III) will be performed in the presence of an organic or mineral base, such as, for example, Et₃N, iPr₂NEt, NaH, Cs₂CO₃ or K₂CO₃, in a polar anhydrous solvent such as THF, DMF or DMSO at a temperature of between −20° C. and 100° C. In the case where Y represents CH₂CO, CH═CHCO or CH₂CH₂CO, L₁ may also represent a hydroxyl. In this case, the reaction with the amine of general formula (III) amounts to the formation of an amide by condensation between this amine and a carboxylic acid derivative. This reaction may be performed by the methods and techniques that are well known to those skilled in the art. One method that is particularly satisfactory consists in condensing a carboxylic acid of general formula (II) with an amine of general formula (III) in the presence of 1-(3-dimethylaminopropyl)-3-ethylcarbodiimide (EDC), 3-hydroxy-1,2,3-benzotriazin-4(3H)-one or a tertiary amine such as diisopropylethylamine, in a polar aprotic solvent such as dichloromethane, at a temperature of between −15° C. and 40° C. In the particular case of the intermediates of formula (IV) in which Y represents (CH₂)ₙ, one preparation method consists in performing a reductive amination using an aldehyde of formula R′₄—Z—(CH₂)ₙ₋₁—CHO in which R′₄ and Z are defined as above, an amine of general formula (III) and a reducing agent such as NaBH₄, NaBH₃CN or NaBH(OAc)₃ in a polar solvent such as 1,2-dichloroethane, THF, DMF or MeOH, at a pH that may be controlled by the addition of an acid, for instance acetic acid, at a temperature of between −20° C. and 100° C.

The intermediate of general formula (IV) is converted into an intermediate of general formula (V) by reaction with W—L₂ in which L₂ may represent a leaving group such as, for example, Cl, Br, I, OSO₂CH₃, OSO₂CF₃ or O-tosyl. In this case, the reaction with the amine of general formula (IV) will be performed in the presence of an organic or mineral base such as, for example, Et₃N, iPr₂NEt, NaH, Cs₂CO₃ or K₂CO₃, in a polar anhydrous solvent such as THF, DMF or DMSO, at a temperature of between −20° and 100° C. The species W—L₂ may also represent an isocyanate or an isothiocyanate. In this case, the reaction with the amine of general formula (IV) will be performed in an apolar solvent such as toluene or benzene, at a temperature of between 40° C. and 100° C. In the particular case of the intermediates of formula (V) in which W represents (CH₂)ₙR₇, one preparation method consists in performing a reductive amination using an aldehyde of formula R₇—(CH₂)ₙ₋₁ECHO in which R₇ is defined as above, an amine of general formula (IV) and a reducing agent such as NaBH₄, NaBH₃CN or NaBH(OAc)₃, in a polar solvent such as 1,2-dichloroethane, THF, DMF or MeOH, at a pH that may be controlled by adding an acid, for instance acetic acid, at a temperature of between −20° C. and 100° C. After deprotection of the intermediate (V) via methods and techniques that are well known to those skilled in the art (“Protective Groups in Organic Synthesis”, T. W. Greene, John Wiley & Sons, 1981 and “Protecting Groups”, P. J. Kocienski, Thieme Verlag, 1994) the intermediate obtained can react with R₅—L₃. L₃ may represent a leaving group such as, for example, Cl, Br, I, OSO₂CH₃, OSO₂CF₃ or O-tosyl. In this case, the reaction with the intermediate secondary amine will be performed in the presence of an organic or mineral base such as, for example, Et₃N, iPr₂NEt, NaH, Cs₂CO₃ or K₂CO₃, in a polar anhydrous solvent such as THF, DMF or DMSO, at a temperature of between −20° and 100° C. In the case where R₅ represents an aromatic radical such as phenyl, L₃ may represent a fluorine. In this case, the reaction with the secondary amine may take place in the presence of a mineral base such as, for example, Cs₂CO₃ or K₂CO₃, in a polar anhydrous solvent such as DMF or DMSO, at a temperature of between 60° C. and 100° C. L₃ may also represent a hydroxyl group. In this case, the reaction with the secondary amine amounts to the formation of an amide by condensation between this amine and a carboxylic acid derivative. This reaction may be performed by the methods and techniques that are well known to those skilled in the art. One method that is particularly satisfactory consists in condensing the carboxylic acid of general formula R₅—L₃ with an amine of general formula (V) in the presence of diisopropylcarbodiimide (DIC) or 3-hydroxy-1,2,3-benzotriazin-4(3H)-one, in a polar aprotic solvent such as DMF, at a temperature of between −15° C. and 40° C. Alternatively, for example, using benzotriazol-1-yloxytris(dimethylamino)phosphonium hexafluorophosphate (BOP) in the presence of 1-hydroxybenzotriazole, a tertiary amine such as diisopropylethylamine, in a polar solvent (DMF or DMSO), at a temperature of between −10° and 35° C. The species R₅—L₃ may also represent an isocyanate or an isothiocyanate. In this case, the reaction with a secondary amine will be performed in an apolar solvent such as toluene or benzene, at a temperature of between 40° and 100° C. The species R₅—L₃ may also represent an anhydride. In this case, the reaction with the secondary amine will be performed in the presence of a tertiary amine such as triethylamine, in a polar solvent (DMF or DMSO) at a temperature between 25° and 100° C. The conversion of R′₄ in the intermediate (VI) into R₄ in the compounds of general formula (I) will be dependent on the nature of R′₄. In the case where R′₄ represents a protecting group, the methods and techniques that are well known to those skilled in the art will be used (“Protective Groups in Organic Synthesis”, T. W. Greene, John Wiley & Sons, 1981 and “Protecting Groups”, P. J. Kocienski, Thieme Verlag, 1994). In the case where R′₄ represents a solid support such as, for example, a trityl resin, cleavage from this solid support may be performed so as to recover the final product. One cleavage method that is particularly suitable consists in treating the intermediate (VI) with trifluoroacetic acid (TFA) in a polar solvent such as dichloromethane, in the presence of triethylsilane, at a temperature of between 0° and 40° C.

Scheme ₂ illustrates the second general process that may be used to prepare the compounds of general formula (I). In the general formulae below, Z, Y, X, R₁, R₂, R₃, R₄, W, R₅, L₁ and L₃ are defined as in the above description. The reaction between the intermediates of general formula (VII) and R₅—L₃ may be performed according to the same procedures as those described in the first process above. The reduction of the nitro function to an amine may be performed by methods and techniques that are well known to those skilled in the art. One method that is particularly satisfactory consists in hydrogenating the compound in the presence of a supported metal catalyst such as palladium-on-charcoal in a polar solvent such as methanol or ethyl acetate, at a temperature of between 20° C. and 35° C. The conversion of the intermediate of formula (VIII) into an intermediate of formula (VI) and then into a compound of general formula (I) may be performed according to the procedures described in the first process above.

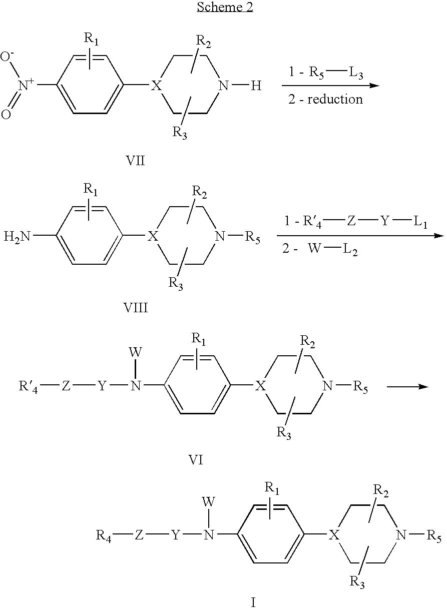

Any method for preparing a compound of general formula (I) starting with another derivative of general formula (I) in which at least one of the substituents is different should also be considered as forming part of the present invention. Thus, for example, a compound of general formula (I) in which Z represents an imidazole and R₄ represents H may be converted into a compound of general formula (I) in which Z represents an imidazole and R₄ represents a benzyl, by selective protection of the imidazole by reaction with trityl chloride followed by a reaction with a benzyl halide according to a method that is well known to those skilled in the art.

It will be understood that in certain chemical reactions or sequences of chemical reactions leading to the preparation of compounds of general formula (I), it is necessary or desirable to protect any sensitive groups in the synthetic intermediates so as to avoid undesirable side reactions. This may be performed by using (introducing and deprotecting) conventional protecting groups such as those described in “Protective Groups in Organic Synthesis”, T. W. Greene, John Wiley & Sons, 1981 and “Protecting Groups”, P. J. Kocienski, Thieme Verlag, 1994. The suitable protecting groups will thus be introduced and removed during the step that is most appropriate to do so and using the methods and techniques described in the references mentioned previously.

When it is desired to isolate a compound of general formula (I) containing at least one basic function in salt form by addition with an acid, this may be achieved by treating the free base of general formula (I) [in which there is at least one basic function] with a suitable acid, preferably in equivalent amount.

When the processes described above for preparing the compounds of the invention give mixtures of diastereoisomers, these isomers may be separated by conventional methods such as preparative chromatography.

When the novel compounds of general formula (I) contain one or more asymmetric centers, they may be prepared in the form of a racemic mixture or in the form of enantiomers, whether by enantioselective synthesis or by resolution.

The examples that follow illustrate the invention without, however, limiting its scope.

### EXAMPLE 1

### (4-{4-[(3H-Imidazol-4-ylmethyl)amino]phenyl}-1-piperazinyl)thiophen-2-ylmethanone

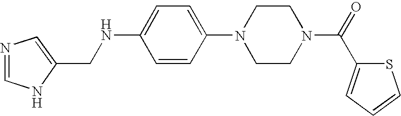

### EXAMPLE 1A

### [4-(4-Nitrophenyl)-1-piperazinyl]thiophen-2-ylmethanone

2-Thiophenecarboxylic acid (6.15 g; 48.0 mmol) dissolved in dichloromethane (200 ml) in the presence of 3-hydroxy-1,2,3-benzotriazin-4(3H)-one (HOOBT) (8.6 g; 52.8 ml) is added to a solution of 1-(4-nitrophenyl)piperazine (10 g; 48 mmol) in dichloromethane (120 ml) in the presence of diisopropylethylamine (DIEA) (8.6 ml; 48 mmol). The mixture is stirred at room temperature for 4 hours and then washed with 2N sodium hydroxide and water. The organic phase is dried over sodium sulfate (Na₂SO₄), filtered and then evaporated to dryness to give a yellow solid (16 g), which is used without further purification for the following step.

¹H NMR, d₆-DMSO (ppm): 3.61 t, 4H; 3.82 s, 4H ; 7.00 dd, 2H; 7.16 dd, 1H ; 7.50 dd, 1H; 7.80 dd, 1H; 8.09 d, 2H

### EXAMPLE 1B

### [4-(4-Aminophenyl)-1-piperazinyl]thiophen-2-ylmethanone

Compound 1A (11.5 g; 36.2 mmol) dissolved in THF (230 ml) in the presence of a catalytic amount of palladium-on-charcoal (at 5%) (505 mg; 0.47 mmol) is hydrogenated under an atmospheric pressure of hydrogen using a balloon. After stirring for 12 hours at room temperature, the medium is filtered through Celite, which is washed with THF. The filtrate is evaporated to dryness to give a beige-colored syrup (10.1 g), which is used without further purification for the following step.

¹H NMR, d₆-DMSO (ppm): 2.94 t, 4H; 3.75 t, 4H; 4.64 s, 2H; 6.50 d, 2H; 6.72 d, 2H; 7.13 dd, 1H; 7.44 dd, 1H; 7.77 dd, 1H.

### EXAMPLE 1C

### 1-Trityl-1H-imidazole-4-carboxaldehyde resin

Trityl chloride resin (2.1 mmol/g) (30 g; 63 mmol) is swollen with CH₂Cl₂ (2×80 ml), followed by addition of a solution of 4(5)-imidazolecarboxaldehyde (18.2 g; 189 mmol) in DMF (134 ml), and then DIEA (134 ml). The mixture is stirred for 36 hours at room temperature and the resin is then filtered off and washed successively with DMF (2×), CH₂Cl₂ (2×), H₂O (2×), MeOH (1×), CH₂Cl₂ (2×) and MeOH (2×).

A sample of this resin (80 mg) is cleaved by treatment with 1/4 TFA/CH₂Cl₂ solution (2 ml) for 10 minutes. After evaporating off the solvents, the product obtained is monitored by HPLC (C18, λ 230 nM, 100% H₂O to 100% CH₃CN (+0.1% TFA) over 25 min) and has a purity of 99%.

### EXAMPLE 1D

### Thiophen-2-yl-(4-{4-[(1-trityl-1H-imidazol-4-ylmethyl)amino]-phenyl}-1-piperazinyl)methanone resin

Resin 1C (75 mg; 0.14 mmol) in dichloromethane (1.5 ml) is treated with compound 1B (68 mg; 0.21 mmol) in the presence of acetic acid (29 μl; 0.56 mmol) and NaBH(OAc)₃ (119 mg; 0.56 mmol). The mixture is stirred at room temperature for 12 hours and the resin is then filtered off and washed with MeOH (2×), H₂O (2×), MeOH (1×), DCM (2×), MeOH (1×) and DCM (1×).

### EXAMPLE 1

### (4-{4-[(3H-Imidazol-4-ylmethyl)amino]phenyl}-1-piperazinyl)thiophen-2-ylmethanon

Resin 1D is cleaved by treatment with 50/50/1 TFA/CH₂Cl₂/Et₃SiH solution (3 ml) for 2 hours. The resin is filtered off and washed with CH₂Cl₂ (2×), and the filtrate is evaporated to dryness to give a colorless syrup (120 mg).

HPLC (C18, λ 220 nM, 100% H₂O to 100% CH₂CN (+0.1% TFA) over 25 min): purity 94%;

¹H NMR, d₆-DMSO (ppm): 3.20 s, 4H; 3.85 s, 4H; 4.31 s, 2H; 6.66 d, 2H; 6.99 d, 2H; 7.15 t, 1H; 7.48 d, 1H; 7.54 s, 1H; 7.79 d, 1H; 9.00 s, 1H; 14,2 broads, 2H.

Mass spectrum (ESI): m/z 368 (MH+).

### EXAMPLE 2

### N-(3H-Imidazol-4-ylmethyl)-N-{4-[4-(2-thiophenecarbonyl)-1-piperazinyl]phenyl}-benzenesulfonamide

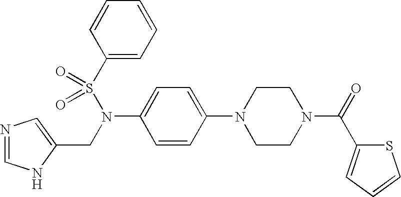

Method A:

Resin 1D (150 mg; 0.266 mmol) is treated with phenylsulfonyl chloride (272 μl; 2.12 mmol) in a 1/1 pyridine/CH₂Cl₂ mixture (6 ml). The mixture is stirred for 6 hours at room temperature and the resin is then filtered off and washed with DMF (3×), MeOH (1×), CH₂Cl₂ (2×), MeOH (1×), H₂O (2×) and MeOH (2×). The resin obtained is then cleaved by treatment with a 50/50/10 TFA/CH₂Cl₂/Et₃SiH mixture (3 ml) for 2.5 hours. The resin is filtered off and washed with CH₂Cl₂ (2×), and the filtrate is then evaporated to dryness to give the desired product (148 mg) in the form of a beige-colored syrup.

Method B:

### EXAMPLE 2A

### 4-(4-Nitrophenyl)piperazine-1-N-9H-fluoren-9-ylmethyloxycarbonyl

1-(4-Nitrophenyl)piperazine (1 g; 4.82 mmol) in aqueous 9% Na₂CO₃ solution (10 ml) is treated at 0° C. with a solution of N-(9-fluorenyl-methoxycarbonyloxy)succinimide (1.79 g; 5.30 mmol) in DMF (12 ml). After stirring for 20 minutes, the mixture is taken up in CH₂Cl₂ and the organic phase is washed with water (2×) and then dried over Na₂SO₄ and evaporated to dryness. The syrup obtained is purified on a column of silica eluted with 3/1 petroleum ether (PE)/ethyl acetate (EtOAc) mixture. The pure product is obtained in the form of a yellow powder (2.04 g; 98%).

¹H NMR, CDCl₃ (ppm): 3.35 broad s, 4H; 3.60 broad d, 4H; 4.26 t, 1H; 4.53 d, 2H; 6.80 d, 2H; 7.32 t, 2H; 7.41 t, 2H; 7.57 d, 2H; 7.77 d, 2H; 8.12 d, 2H;

| | | | | |
|---|---|---|---|---|
| Elemental analysis | % calculated: | C 69.92; | H 5.40; | N 9.78 |
| (C₂₅H₂₃N₃O₄) | % found: | C 69.58; | H 5.30; | N 9.73 |

### EXAMPLE 2B

### 4-(4-Aminophenyl)piperazine-1-N-9H-fluoren-9-ylmethloxycarbonyl

Compound 2A (2.04 g; 4.75 mmol) dissolved in a 4/1 MeOH/THF mixture (80 ml) in the presence of a catalytic amount of palladium-on-charcoal (at 5%) (25 mg; 0.03 mmol) is hydrogenated under an atmospheric pressure of hydrogen using a balloon. After stirring for 12 hours at room temperature, the medium is filtered through Celite, which is washed with THF. The filtrate is evaporated to dryness to give a beige-colored syrup (1.89 g; 100%), which is used without purification for the following step.

¹H NMR, CDCl₃ (ppm): 2.96 broad s, 4H; 3.61 broad s, 4H; 4.26 t, 1H; 4.46 d, 2H; 6.65 d, 2H; 6.80 d, 2H; 7.30 t, 2H; 7.38 t, 2H; 7.58 d, 2H; 7.76 d, 2H.

### EXAMPLE 2C

### 4-{4-[(1-Trityl-1H-imidazol-4-ylmethyl)amino]phenyl}piperazine-1-N-9H-fluoren-9-ylmethyloxycarbonyl resin

Compound 2C is prepared from resin 1C (600 mg; 1.87 mmol/g; 1.12 mmol) and compound 2B (1.34 g; 3.36 mmol) according to the conditions used for the preparation of Example 1D. The resin obtained (974 mg) is monitored by HPLC analysis of a sample after cleavage 1/4 TFA/CH₂Cl₂) and has a purity of 96%.

### EXAMPLE 2D

### 4-{4-[Benzenesulfonyl-(1-trityl-1H-imidazol-4-ylméthyl)amino]phenyl}piperazine-1-N-9H-fluoren-9-ylmethyloxycarbonyl resin

Resin 2C (11.16 g; 1.09 mmol/g; 12.16 mmol) is swollen with CH₂Cl₂ (2×50 ml) and is then suspended in pyridine (200 ml) and treated with phenylsulfonyl chloride (17.3 ml; 97.3 mmol) at room temperature for 6 hours. The resin is then filtered off and washed successively with DMF (3×); MeOH (1×); CH₂Cl₂ (1×), MeOH (1×), H₂O (2×) and MeOH (2×). The resin obtained is monitored by HPLC analysis of a sample after cleavage (1/4 TFA/CH₂Cl₂) and has a purity of 94%.

¹H NMR, d₆-DMSO (ppm) of the cleaved product: 3.04 broad s, 4H; 3.40 broad s, 4H; 4.28 t, 1H; 4.41 d, 2H; 4.82 s, 2H; 6.78 q, 4H; 7.35 t, 2H; 7.41 m, 3H; 7.63 m, 5H; 7.74 m, 1H; 7.89 d, 2H; 8.97 s, 1H;

Mass spectrum (ESI): m/z 620 (MH⁺).

### EXAMPLE 2E

### N-(4-1-piperazinyl-phenyl)-N-(1-trityl-1H-imidazol-4-ylmethyl)-benzenesulfonamide resin

Resin 2D (4.4 g, 0.95 mmol/g, 4.18 mmol) is deprotected by treatment with a 1/4 piperidine/DMF solution (50 ml) for 2 hours. The resin is then filtered off and washed successively with DMF (3×), MeOH (2×), CH₂Cl₂ (2×), DMF (1×) and MeOH (2×).

### EXAMPLE 2

### N-(3H-imidazol-4-ylmethyl)-N-{4-[4-(₂-thiophenecarbonyl)-1-piperazinyl]pheny}benzenesulfonamide

Resin 2E (75 mg; 1.11 mmol/g; 0.08 mmol) suspended in CH₂Cl₂ (3 ml) in the presence of diisopropylethylamine (DIEA) (64 μl; 0.32 mmol) is treated at room temperature with 2-thiophenecarbonyl chloride (34 μl; 0.32 mmol). After stirring for 4 hours, the resin is filtered off and then washed successively with DMF (3×), MeOH (1×), CH₂Cl₂ (2×), MeOH (1×), H₂O (2×) and MeOH (2×). The resin is then cleaved by treatment with 5/5/1 TFA/CH₂Cl₂/Et₃SiH mixture (3 ml) for _{2.5} hours to give the expected product after evaporation of the filtrate.

¹H NMR, d₆-DMSO (ppm): 3.21 broad s, 4H; 3.74 broad s, 4H; 4.82 s, 2H; 6.82 m, 4H; 7.14 t, 1H; 7.41 s, 1H; 7.44 d, 1H; 7.62 m, 4H; 7.74 m, 2H; 8.97 s, 1H Mass spectrum (ESI): m/z 508 (MH⁺).

### EXAMPLE 3

### N-(3H-Imidazol-4-ylmethyl)-N-(4-1-piperazinylphenyl)benzenesulfonamide

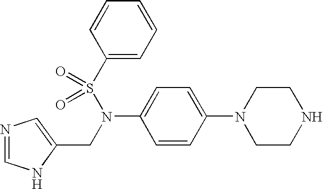

Resin 2E (50 mg; 1.11 mmol/g; 0.06 mmol) is cleaved by treatment with a 5/5/1 TFA/CH₂Cl₂/Et₃SiH mixture (3 ml) for 2.5 hours to give compound 3 (37 mg; 91%).

¹H NMR, d₆-DMSO (ppm): 3.19, broad s, 4H; 3.31 m, 4H; 4.83 s, ₂H; 6.85 m, 4H; 7.40 s, 1H; 7.65 m, 4H; 7.74 m, 1H; 8.88 broad s, 1H; 8.94 s, 1H Mass spectrum (ESI): m/z 398 (MH⁺).

### EXAMPLES 4 TO 11

Compounds 4 to 11 were synthesized under the same conditions as those described for the preparation of Example 2, starting with resin 2E (Method B).

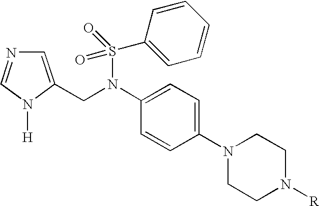

| | | | Mass spectrum |
|---|---|---|---|
| Example | R | Compound name | (M⁺H)⁺ |
| 4 | 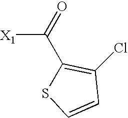 | N-{4-[4-(3-Chloro-2- thiophenecarbonyl)-1- piperazinyl]phenyl}-N-(3H-imidazol- 4-ylmethyl)-benzenesulfonamide | 543 |
| 5 | 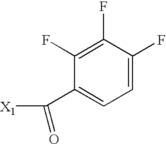 | N-(3H-Imidazol-4-ylmethyl)-N-{4- [4-(2,3,4-trifluorobenzoyl)-1- piperazinyl]phenyl}benzenesulfonamide | 556 |
| 6 | 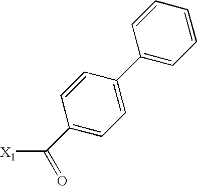 | N-{4-[4-(Biphenyl-4-carbonyl)-1- piperazinyl]phenyl}-N-(3H-imidazol- 4-ylmethyl)-benzenesulfonamide | 578 |
| 7 | 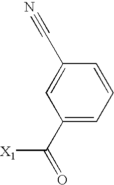 | N-{4-[4-(2,6-Dimethoxy-benzoyl)-1- piperazinyl]phenyl}-N-(3H-imidazol- 4-ylmethyl)-benzenesulfonamide | 527 |
| 8 | 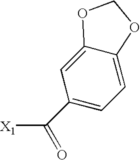 | N-{4-[4-(Benzo[1,3]dioxole-5- carbonyl)-1-piperazinyl]phenyl}-N- (3H-imidazol-4-ylmethyl)- benzenesulfonamide | 546 |
| 9 | 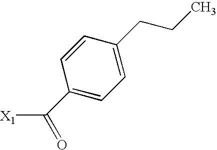 | N-(3H-Imidazol-4-ylmethyl)-N-{4- [4-(4-propylbenzoyl)-1- piperazinyl]phenyl}benzenesulfonamide | 544 |
| 10 | 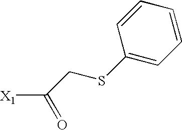 | N-(3H-Imidazol-4-ylmethyl)-N-{4- [4-(2-phenylsulfanylacetyl)-1- piperazinyl]phenyl}benzenesulfonamide | 548 |
| 11 | 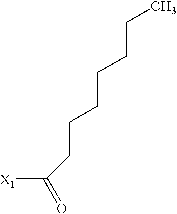 | N-(3H-Imidazol-4-ylmethyl)-N-[4-(4- octanoyl-1-piperazinyl)phenyl]- benzenesulfonamide | 524 |

### EXAMPLES 12 TO 25

Compounds 12 to 25 were synthesized according to the following general procedure:

Resin 2E (75 mg; 1.11 mmol/g; 0.08 mmol) suspended in DMF (3 ml) in the presnce of carboxylic acid (0.32 mmol) is treated with benzotriaol-1-yloxytris(dimethylamino)phosphonium hexafluorophosphate (BOP) (177 mg; 0.4 mmol), N-hydroxybenzotriazole (HOBt) (54 mg; 0.4 mmol) and DIEA (69 μl; 0.4 mmol) at room temperature for 7 hours. The resin is filtered off and then washed successively with DMF (3×), MeOH (1×), CH₂Cl₂ (2×), MeOH (1×), H₂O (2×) and MeOH (2×). The resin is then cleaved by treatment with a 5/5/1 TFA/CH₂Cl₂/Et₃SiH mixture (3 ml) for 2.5 hours, to give the expected product after evaporation of the filtrate.

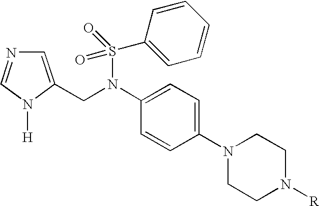

| | | | Mass spectrum |
|---|---|---|---|
| Example | R | Compound name | (M⁺H)⁺ |
| 12 | 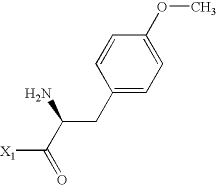 | N-(4-{4-[2-Amino-3-(4-methoxy-phenyl)- propionyl]-1-piperazinyl}phenyl)-N-(3H- imidazol-4-ylmethyl)benzenesulfonamide | 575 |
| 13 | 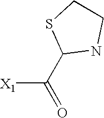 | N-(3H-Imidazol-4-ylmethyl)-N-{4-[4- (thiazolidine-2-carbonyl)-1- piperazinyl]phenyl}benzenesulfonamide | 513 |
| 14 | 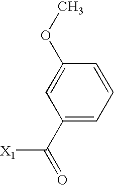 | N-(3H-Imidazol-4-ylmethyl)-N-{4-[4-(3- methoxybenzoyl)-1-piperazinyl]phenyl}- benzenesulfonamide | 532 |
| 15 | 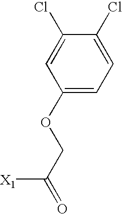 | N-(4-{4-[2-(3,4-Dichlorophenoxy)acetyl]- 1-piperazinyl}phenyl)-N-(3H-imidazol-4- ylmethyl)benzenesulfonamide | 601 |
| 16 | 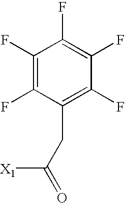 | N-(3H-Imidazol-4-ylmethyl)-N-{4-[4-(2- pentafluorophenylacetyl)-1- piperazinyl]phenyl}benzenesulfonamide | 606 |
| 17 | 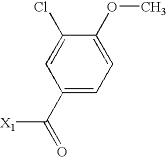 | N-{4-[4-(3-Chloro-4-methoxy-benzoyl)-1- piperazinyl]phenyl}-N-(3H-imidazol-4- ylmethyl)benzenesulfonamide | 567 |
| 18 | 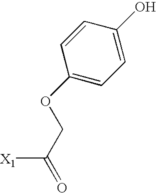 | N-(4-{4-[2-(4-Hydroxyphenoxy)acetyl]-1- piperazinyl}phenyl)-N-(3H-imidazol-4- ylmethyl)benzenesulfonamide | 548 |
| 19 | 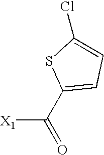 | N-{4-[4-(5-Chloro-2-thiophenecarbonyl)- 1-piperazinyl]phenyl}-N-(3H-imidazol-4- ylmethyl)benzenesulfonamide | 543 |
| 20 | 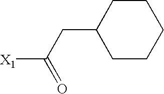 | N-{4-[4-(2-Cyclohexylacetyl)-1- piperazinyl]phenyl}-N-(3H-imidazol-4- ylmethyl)-benzenesulfonamide | 522 |
| 21 | 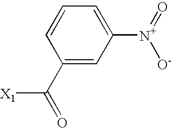 | N-(3H-Imidazol-4-ylmethyl)-N-{4-[4-(3- nitrobenzoyl)-1-piperazinyl]phenyl}- benzenesulfonamide | 547 |
| 22 | 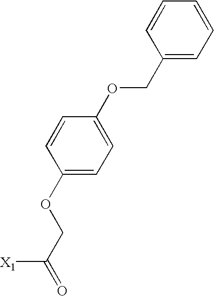 | N-(4-{4-[2-(4-Benzyloxyphenoxy)-acetyl]- 1-piperazinyl}phenyl)-N-(3H-imidazol-4- ylmethyl)benzenesulfonamide | 638 |
| 23 | 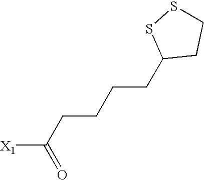 | N-{4-[4-(5-[1,2]Dithiolan-3-yl-pentanoyl)- 1-piperazinyl]phenyl}-N-(3H-imidazol-4- ylmethyl)benzenesulfonamide | 586 |
| 24 | 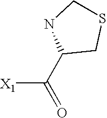 | N-(3H-Imidazol-4-ylmethyl)-N-{4-[4- (thiazolidine-4-carbonyl)-1- piperazinyl]phenyl}benzenesulfonamide | 513 |
| 25 | 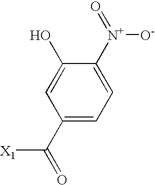 | N-{4-[4-(3-Hydroxy-4-nitrobenzoyl)-1- piperazinyl]phenyl}-N-(3H-imidazol-4- ylmethyl)-benzenesulfonamide | 563 |

### EXAMPLES 26 TO 39

Compounds 26 to 39 were synthesized according to the following general procedure:

Resin 2E (75 mg; 1.11 mmo1′g, 0.08 mmol) suspended in toluene (4 ml) is treated with an isocyanate (0.32 mmol) at 50° C. for 4 hours. The resin is filtered off and then washed successively with DMF (3×), MeOH (1×), CH₂Cl₂ (2×), MeOH (1×), H₂O (2×) and MeOH (2×). The resin is then cleaved by treatment with a 5/5/1 TFA/CH₂Cl₂/Et₃SiH mixture (3 ml) for 2.5 hours, to give the expected product after evaporation of the filtrate.

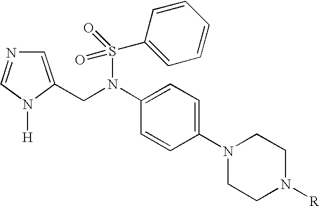

| | | | Mass spectrum |
|---|---|---|---|
| Example | R | Compound name | (M⁺H)⁺ |
| 26 | 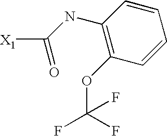 | 4-{4-[Benzenesulfonyl-(3H- imidazol-4-ylmethyl)amino]phenyl}- piperazine-1-carboxylic acid (2-trifluoromethoxyphenyl)amide | 601 |
| 27 | 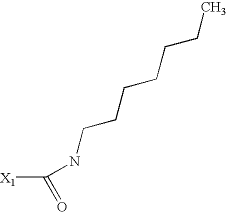 | 4-{4-[Benzenesulfonyl-(3H- imidazol-4-ylmethyl)amino]phenyl}- piperazine-1-carboxylic acid heptylamide | 539 |
| 28 | 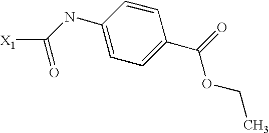 | 4-[(4-{4-[Benzenesulfonyl-(3H- imidazol-4-ylmethyl)amino]- phenyl}piperazine-1- carbonyl)amino]benzoic acid ethyl ester | 589 |
| 29 | 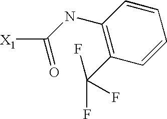 | 4-{4-[Benzenesulfonyl-(3H- imidazol-4-ylmethyl)amino]- phenyl}piperazine-1-carboxylic acid (2-trifluoromethylphenyl)amide | 585 |
| 30 | 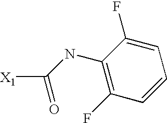 | 4-{4-[Benzenesulfonyl-(3H- imidazol-4-ylmethyl)amino]phenyl}- piperazine-1-carboxylic acid (2,6- difluorophenyl)amide | 553 |
| 31 | 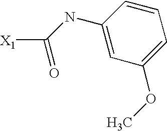 | 4-{4-[Benzenesulfonyl-(3H- imidazol-4-ylmethyl)amino]- phenyl}piperazine-1-carboxylic acid (3-methoxyphenyl)-amide | 547 |
| 32 | 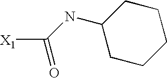 | 4-{4-[Benzenesulfonyl-(3H- imidazol-4-ylmethyl)amino]phenyl}- piperazine-1-carboxylic acid phenylamide | 517 |
| 33 | 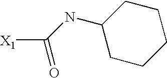 | 4-{4-[Benzenesulfonyl-(3H- imidazol-4-ylmethyl)amino]phenyl}- piperazine-1-carboxylic acid cyclohexylamide | 523 |
| 34 | 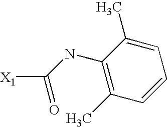 | 4-{4-[Benzenesulfonyl-(3H- imidazol-4-ylmethyl)amino]phenyl}- piperazine-1-carboxylic acid (2,6-dimethylphenyl)amide | 545 |
| 35 | 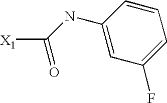 | 4-{4-[Benzenesulfonyl-(3H- imidazol-4-ylmethyl)amino]phenyl}- piperazine-1-carboxylic acid (3-fluorophenyl)amide | 535 |
| 36 | 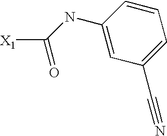 | 4-{4-[Benzenesulfonyl-(3H- imidazol-4-ylmethyl)amino]phenyl}- piperazine-1-carboxylic acid (3-cyanophenyl)amide | 542 |
| 37 | 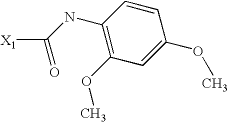 | 4-{4-[Benzenesulfonyl-(3H- imidazol-4-ylmethyl)amino]phenyl}- piperazine-1-carboxylic acid (2,4-dimethoxyphenyl)amide | 577 |
| 38 | 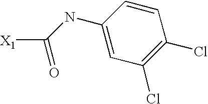 | 4-{4-[Benzenesulfonyl-(3H- imidazol-4-ylmethyl)amino]phenyl}- piperazine-1-carboxylic acid (3,4-dichlorophenyl)amide | 586 |
| 39 | 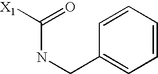 | 4-{4-[Benzenesulfonyl-(3H- imidazol-4-ylmethyl)amino]phenyl}- piperazine-1-carboxylic acid benzylamide | 531 |

### EXAMPLES 40 TO 47

Compounds 40 to 47 were synthesized according to the following general procedure:

Resin 2E (75 mg; 0.8 mmol/g; 0.06 mmol) suspended in toluene (4 ml) is treated with an isothiocyanate (0.24 mmol) at 50° C. for 4 hours. The resin is filtered off and then washed successively with DMF (3×), MeOH (1×), CH₂Cl₂ (2×), MeOH (1×), H₂O (2×) and MeOH (2×). The resin is then cleaved by treatment with a 5/5/1 TFA/CH₂Cl₂/Et₃SiH mixture (3 ml) for 2.5 hours, to give the expected product after evaporation of the filtrate.

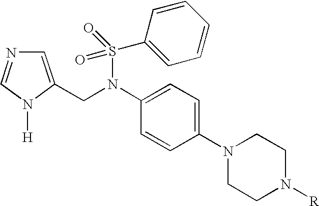

| | | | Mass spectrum |
|---|---|---|---|
| Example | R | Compound name | (M + H)+ |
| 40 | 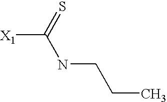 | 4-{4-[Benzenesulfonyl-(3H-imidazol- 4-ylmethyl)amino]phenyl}-1- piperazinecarbothioic acid propylamide | 499 |
| 41 | 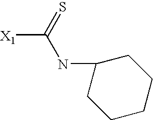 | 4-{4-[Benzenesulfonyl-(3H-imidazol- 4-ylmethyl)amino]phenyl}-1- piperazinecarbothioic acid cyclohexylamide | 539 |
| 42 | 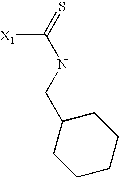 | 4-{4-[Benzenesulfonyl-(3H-imidazol- 4-ylmethyl)amino]phenyl}-1- piperazinecarbothioic acid cyclohexylmethylamide | 553 |
| 43 | 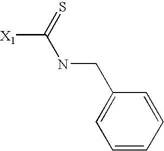 | 4-{4-[Benzenesulfonyl-(3H-imidazol- 4-ylmethyl)amino]phenyl}-1- piperazinecarbothioic acid benzylamide | 547 |
| 44 | 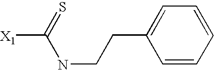 | 4-{4-[Benzenesulfonyl-(3H-imidazol- 4-ylmethyl)amino]phenyl}-1- piperazinecarbothioic acid phenethylamide | 561 |
| 45 | 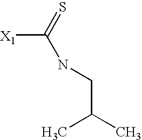 | 4-{4-[Benzenesulfonyl-(3H-imidazol- 4-ylmethyl)amino]phenyl}-1- piperazinecarbothioic acid isobutylamide | 513 |
| 46 | 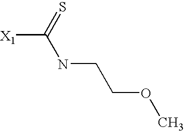 | 4-{4-[Benzenesulfonyl-(3H-imidazol- 4-ylmethyl)amino]phenyl}-1- piperazinecarbothioic acid (2-methoxyethyl)amide | 515 |
| 47 | 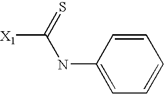 | 4-{4-[Benzenesulfonyl-(3H-imidazol- 4-ylmethyl)amino]phenyl}-1- piperazinecarbothioic acid phenylamide | 533 |

### EXAMPLES 48 TO 56

Compounds 48 to 56 were synthesized according to the following general procedure:

Resin 1D (50 mg; 1.77 mmol/g; 0.084 mmol) suspended in pyridine (3 ml) is treated at room temperature with an acid chloride (0.336 mmol) for 7 hours. The resin is filtered off and then washed successively with DMF (3×), MeOH (1×), CH₂Cl₂ (2×), MeOH (1×), H₂O (2×) and MeOH (2×). The resin is then cleaved by treatment with a 5/5/1 TFA/CH₂Cl₂/Et₃SiH mixture (3 ml) for 2.5 hours, to give the expected product after evaporation of the filtrate.

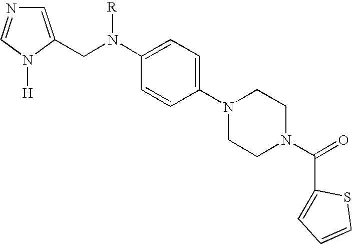

| | | | Mass spectrum |
|---|---|---|---|
| Example | R | Compound name | (M + H)+ |
| 48 | 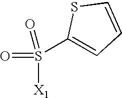 | Thiophene-2-sulfonic acid (3H- imidazol-4-ylmethyl)-{4-[4-(thiophene- 2-carbonyl)-1-piperazinyl]phenyl}amide | 514 |
| 49 | 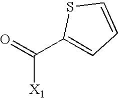 | Thiophene-2-carboxylic acid (3H- imidazol-4-ylmethyl)-{4-[4-(thiophene- 2-carbonyl)-1-piperazinyl]phenyl}amide | 478 |
| 50 | 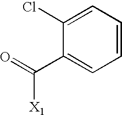 | 2-Chloro-N-(3H-imidazol-4-ylmethyl)- N-{4-[4-(thiophene-2-carbonyl)-1- piperazinyl]phenyl}benzamide | 506 |
| 51 | 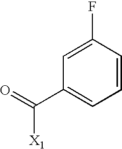 | 3-Fluoro-N-(3H-imidazol-4-ylmethyl)- N-{4-[4-(thiophene-2-carbonyl)-1- piperazinyl]phenyl}-benzamide | 490 |
| 52 | 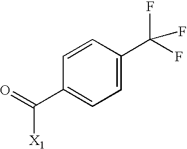 | N-(3H-Imidazol-4-ylmethyl)-N-{4-[4- (thiophene-2-carbonyl)-1- piperazinyl]phenyl}-4- trifluoromethylbenzamide | 540 |
| 53 | 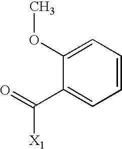 | N-(3H-Imidazol-4-ylmethyl)-2- methoxy-N-{4-[4-(thiophene-2- carbonyl)-1-piperazinyl]phenyl}- benzamide | 502 |
| 54 | 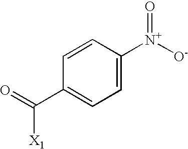 | N-(3H-Imidazol-4-ylmethyl)-4-nitro-N- {4-[4-(thiophene-2-carbonyl)-1- piperazinyl]phenyl}benzamide | 517 |
| 55 | 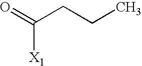 | N-(3H-Imidazol-4-ylmethyl)-N-{4-[4- (thiophene-2-carbonyl)-1- piperazinyl]phenyl}butyramide | 438 |
| 56 | 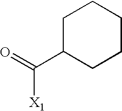 | Cyclohexanecarboxylic acid (3H- imidazol-4-ylmethyl)-{4-[4-(thiophene- 2-carbonyl)-1-piperazinyl]phenyl}amide | 478 |

### EXAMPLE 57

### (4-{4-{(3H-Imidazol-4-ylmethyl)amino]phenyl}-1-piperazinyl)-(5-chloro-2-thiophenyl)methanone

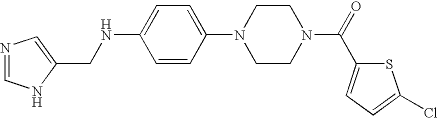

### EXAMPLE 57A

### [4-(4-Aminophenyl)-1-piperazinyl](5-chloro-2-thiophenyl)-methanone

Compound 57A is prepared from 5-chloro-2-thiophenecarboxylic acid (7.8 g; 4.8 mmol) and 1-(4-nitrophenyl)piperazine (10 g; 4.8 mmol) according to the conditions used for the preparation of Example 1B.

¹H NMR, d₆-DMSO (ppm): 2.94 t, 4H; 3.74 broad s, 4H; 4.64 s, 2H; 6.49 d, 2H; 6.49 d, 2H; 6.71 d, 2H; 7.19 d, 1H; 7.35 d, 1H;

Mass spectrum (ESI): m/z 322 (MH⁺).

### EXAMPLE 57B

### 5-Chloro-2-thiophenyl-(4-{4-[(1-trityl-1H-imidazol-4ylmethyl)amino]phenyl}-1-piperazinyl)methanone resin

Resin 57B is prepared from compound 57A (2.1 g; 6.45 mmol) and resin 1C (3 g; 1.075 mmol/g; 3.22 mmol) according to the conditions used for the preparation of Example 1D.

### EXAMPLE 57-5

### Chloro-2-thiophenyl-(4-{4-[(1-trityl-1H-imidazol-4-ylmethyl)-amino]phenyl}1-piperazinyl)methanone resin

Resin 57B (100 mg) is cleaved under the conditions used for the preparation of Example 1, starting with 1D to give a colorless syrup (46 mg).

HPLC (C18, 220 nm, 100% H₂O to 100% CH₃CN (+0.1% TFA) in 10 min): purity 99% ¹H NMR, d₆-DMSO (ppm): 3.16 broad s, 4H; 3.83 broad s, 4H; 4.31 s, 2H; 6.65 d, 2H; 6.99 m, 2H; 7.18 d, 1H; 7.38 d, 1H; 7.54 s, 1H; 9.00 d, 1H;

Mass spectrum (ESI): m/z 402 (MH⁺).

### EXAMPLES 58 TO 77

Compounds 58 to 77 were synthesized from resin 57B (100 mg, 0.62 mmol/g, 0.62 mmol) and from the acid chloride (58 to 73) or sulfonyl chloride (74 to 77) (0.24 mmol) according to the conditions used for the preparation of Examples 48 to 56.

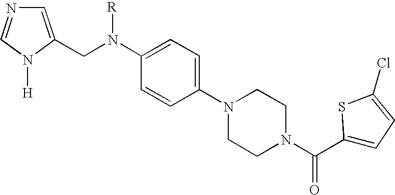

| | | | Mass spectrum |
|---|---|---|---|
| Example | R | Compound name | (M + H)+ |
| 58 | 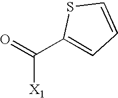 | Thiophene-2-carboxylic acid {4-[4-(5- Chloro-2-thiophenecarbonyl)-1- piperazinyl]phenyl}(3H-imidazol-4- ylmethyl)amide | 512 and 514 |
| 59 | 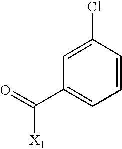 | 3-Chloro-N-{4-[4-(5-Chloro-2- thiophenecarbonyl)-1- piperazinyl]phenyl}-N-(3H-imidazol- 4-ylmethyl)benzamide | 540 and 542 |
| 60 | 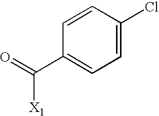 | 4-Chloro-N-{4-[4-(5-Chloro-2- thiophenecarbonyl)-1- piperazinyl]phenyl}-N-(3H-imidazol- 4-ylmethyl)benzamide | 540 and 542 |
| 61 | 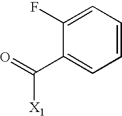 | N-{4-[4-(5-Chloro-2- thiophenecarbonyl)-1- piperazinyl]phenyl}-2-fluoro-N-(3H- imidazol-4-ylmethyl)benzamide | 524 and 526 |
| 62 | 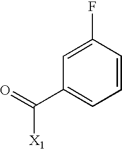 | N-{4-[4-(5-Chloro-2- thiophenecarbonyl)-1- piperazinyl]phenyl}-3-fluoro-N-(3H- imidazol-4-ylmethyl)benzamide | 524 and 526 |
| 63 | 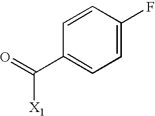 | N-{4-[4-(5-Chloro-2- thiophenecarbonyl)-1- piperazinyl]phenyl}-4-fluoro-N-(3H- imidazol-4-ylmethyl)benzamide | 524 et 526 |
| 64 | 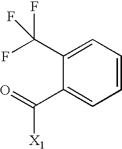 | N-{4-[4-(5-Chloro-2- thiophenecarbonyl)-1- piperazinyl]phenyl}-N-(3H-imidazol- 4-ylmethyl)-2-trifluoromethylbenzamide | 574 and 576 |
| 65 | 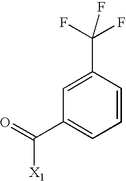 | N-{4-[4-(5-Chloro-2- thiophenecarbonyl)-1- piperazinyl]phenyl}-N-(3H-imidazol- 4-ylmethyl)-3- trifluoromethylbenzamide | 574 and 576 |
| 66 | 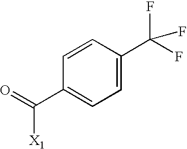 | N-{4-[4-(5-Chloro-2- thiophenecarbonyl)-1- piperazinyl]phenyl}-N-(3H-imidazol- 4-ylmethyl)-4-trifluoromethylbenzamide | 574 and 576 |
| 67 | 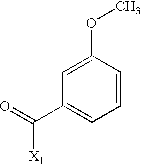 | N-{4-[4-(5-Chloro-2- thiophenecarbonyl)-1- piperazinyl]phenyl}-N-(3H-imidazol- 4-ylmethyl)-3-methoxybenzamide | 536 and 538 |
| 68 | 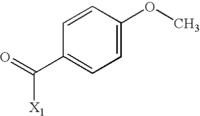 | N-{4-[4-(5-Chloro-2- thiophenecarbonyl)-1- piperazinyl]phenyl}-N-(3H-imidazol- 4-ylmethyl)-4-methoxybenzamide | 536 and 538 |
| 69 | 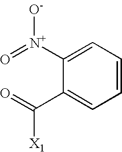 | N-{4-[4-(5-Chloro-2- thiophenecarbonyl)-1- piperazinyl]phenyl}-N-(3H-imidazol- 4-ylmethyl)-2-nitrobenzamide | 551 and 553 |
| 70 | 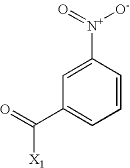 | N-{4-[4-(5-Chloro-2- thiophenecarbonyl)-1- piperazinyl]phenyl}-N-(3H-imidazol- 4-ylmethyl)-3-nitrobenzamide | 551 and 553 |
| 71 | 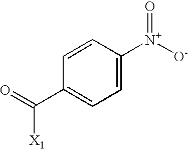 | N-{4-[4-(5-Chloro-2- thiophenecarbonyl)-1- piperazinyl]phenyl}-N-(3H-imidazol- 4-ylmethyl)-4-nitrobenzamide | 551 and 553 |
| 72 | 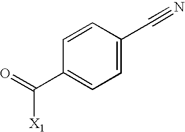 | N-{4-[4-(5-Chloro-2- thiophenecarbonyl)-1- piperazinyl]phenyl}-4-cyano-N-(3H- imidazol-4-ylmethyl)benzamide | 531 and 533 |
| 73 | 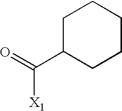 | Cyclohexanecarboxylic acid {4-[4-(5- Chloro-2-thiophenecarbonyl)-1- piperazinyl]phenyl}-(3H-imidazol-4- ylmethyl)amide | 512 and 514 |
| 74 | 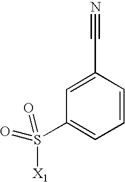 | N-{4-[4-(5-Chloro-2- thiophenecarbonyl)-1- piperazinyl]phenyl}-3-cyano-N-(3H- imidazol-4- ylmethyl)benzenesulfonamide | 567 and 569 |
| 75 | 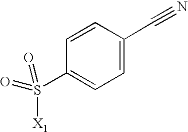 | N-{4-[4-(5-Chloro-2- thiophenecarbonyl)-1- piperazinyl]phenyl}-4-cyano-N-(3H- imidazol-4- ylmethyl)benzenesulfonamide | 567 and 569 |
| 76 | 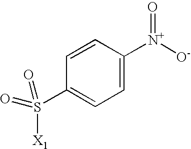 | N-{4-[4-(5-Chloro-2- thiophenecarbonyl)-1- piperazinyl]phenyl}-N-(3H-imidazol- 4-ylmethyl)-4- nitrobenzenesulfonamide | 587 and 589 |
| 77 | 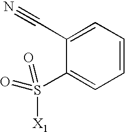 | N-{4-[4-(5-Chloro-2- thiophenecarbonyl)-1- piperazinyl]phenyl}-2-cyano-N-(3H- imidazol-4- ylmethyl)benzenesulfonamide | 567 and 569 |

### EXAMPLES 78 TO 85

Compounds 78 to 85 were synthesized from resin 57B (75 mg; 1.22 mmol/g, 0.092 mmol) and from isocyanates (0.37 mmol) according to the conditions used for the preparation of Examples 26 to 39.

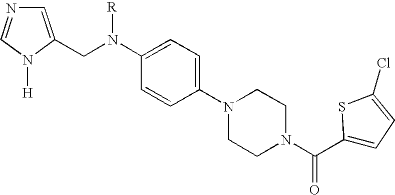

| | | | Mass spectrum |
|---|---|---|---|
| Example | R | Compound name | (M + H)+ |
| 78 | 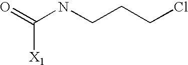 | 3-(3-Chloropropyl)-1-{4-[4-(5-chloro- thiophene-2-carbonyl)-1- piperazinyl]phenyl}-1-(3H-imidazol-4- ylmethyl)urea | 521 and 523 |
| 79 | 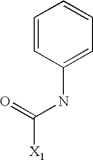 | 1-{4-[4-(5-Chloro-2- thiophenecarbonyl)-1- piperazinyl]phenyl}-1-(3H-imidazol-4- ylmethyl)-3-phenylurea | 521 and 523 |
| 80 | 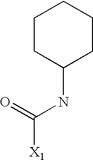 | 1-{4-[4-(5-Chloro-2- thiophenecarbonyl)-1- piperazinyl]phenyl}-3-cyclohexyl-1- (3H-imidazol-4-ylmethyl)urea | 527 and 529 |
| 81 | 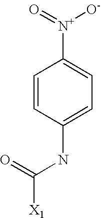 | 1-{4-[4-(5-Chloro-2- thiophenecarbonyl)-1- piperazinyl]phenyl}-1-(3H-imidazol-4- ylmethyl)-3-(4-nitro-phenyl)urea | 566 and 568 |
| 82 | 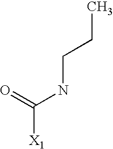 | 1-{4-[4-(5-Chloro-2- thiophenecarbonyl)-1- piperazinyl]phenyl}-1-(3H-imidazol-4- ylmethyl)-3-propyl-urea | 487 and 489 |
| 83 | 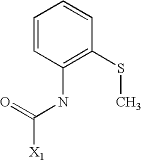 | 1-{4-[4-(5-Chloro-2- thiophenecarbonyl)-1- piperazinyl]phenyl}-1-(3H-imidazol-4- ylmethyl)-3-(2-methylsulfanylphenyl)urea | 567 and 569 |
| 84 | 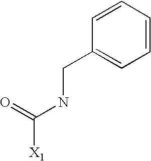 | 3-Benzyl-1-{4-[4-(5-Chloro-2- thiophenecarbonyl)-1- piperazinyl]phenyl}-1-(3H-imidazol-4- ylmethyl)urea | 535 and 537 |
| 85 | 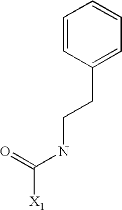 | 1-{4-[4-(5-Chloro-2- thiophenecarbonyl)-1- piperazinyl]phenyl}-1-(3H-imidazol-4- ylmethyl)-3-phenethylurea | 549 and 551 |

### EXAMPLES 86 TO 90

Compounds 86 to 90 were synthesized using resin 57B (75 mg; 1.22 mmol/g; 0.092 mmol) and isothiocyanates (0.37 mmol) according to the conditions used for the preparation of Examples 40 to 47.

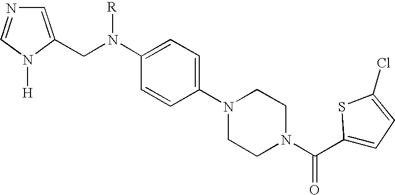

| | | | Mass spectrum |
|---|---|---|---|
| Example | R | Compound name | (M + H)+ |
| 86 | 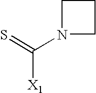 | Azetidine-1-carbothioic acid {4-[4-(5- Chloro-2-thiophenecarbonyl)-1- piperazinyl]phenyl}(3H-imidazol-4- ylmethyl)amide | 501 and 503 |
| 87 | 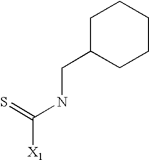 | 1-{4-[4-(5-Chloro-2-thiophenecarbonyl)-1- piperazinyl]phenyl}-3-cyclohexylmethyl- 1-(3H-imidazol-4-ylmethyl)thiourea | 557 and 559 |
| 88 | 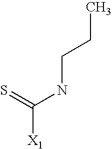 | 1-{4-[4-(5-Chloro-2-thiophenecarbonyl)-1- piperazinyl]phenyl}-1-(3H-imidazol-4- ylmethyl)-3-propylthiourea | 503 and 505 |
| 89 | 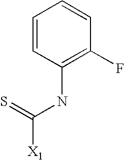 | 1-{4-[4-(5-Chloro-2-thiophenecarbonyl)-1- piperazinyl]phenyl}-3-(2-fluorophenyl)-1- (3H-imidazol-4-ylmethyl)thiourea | 555 and 557 |
| 90 | 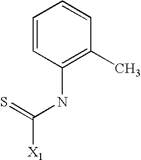 | 1-{4-[4-(5-Chloro-2-thiophenecarbonyl)-1- piperazinyl]phenyl}-1-(3H-imidazol-4- ylmethyl)-3-o-tolylthiourea | 551 and 553 |

### EXAMPLES 91 TO 100

### EXAMPLE 91A

### 4-Nitro-N-(4-1-piperazinylphenyl)-N-(1-trityl-1H-imidazol-4-ylmethyl)benzamide resin

Resin 91A is prepared from resin 2C (2.6 g; 1.03 mmol/g; 2.68 mmol) and 4-nitrobenzoyl chloride (2 g; 2.7 mmol) according to the conditions used for the resin 2E.

### EXAMPLES 91 TO 100

Compounds 91 to 100 were synthesized using resin 91A (80 mg; 1.1 mmol/g; 0.096 mmol) and isothiocyanates (0.38 mmol) according to the conditions used for the preparation of Examples 40 to 47.

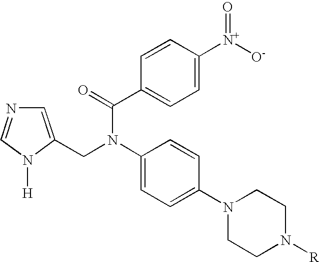

| | | | Mass spectrum |
|---|---|---|---|
| Example | R | Compound name | (M + H)+ |
| 91 | 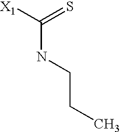 | N-(3H-Imidazol-4-ylmethyl)-4-nitro- N-[4-(4-propylthiocarbamoyl-1- piperazinyl)phenyl]benzamide | 508 |
| 92 | 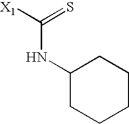 | N-[4-(4-Cyclohexylthiocarbamoyl-1- piperazinyl)-phenyl]-N-(3H- imidazol-4-ylmethyl)-4-nitrobenzamide | 548 |
| 93 | 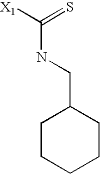 | N-{4-[4-(Cyclohexylmethyl- thiocarbamoyl)-1- piperazinyl]phenyl}-N-(3H- imidazol-4-ylmethyl)-4-nitrobenzamide | 562 |
| 94 | 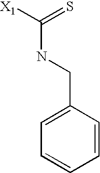 | N-[4-(4-Benzylthiocarbamoyl-1- piperazinyl)phenyl]-N-(3H-imidazol- 4-ylmethyl)-4-nitrobenzamide | 556 |
| 95 | 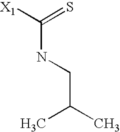 | N-(3H-Imidazol-4-ylmethyl)-N-[4- (4-isobutylthiocarbamoyl-1- piperazinyl)phenyl]-4-nitrobenzamide | 522 |
| 96 | 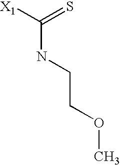 | N-(3H-Imidazol-4-ylmethyl)-N-{4- [4-(2-methoxyethylthiocarbamoyl)-1- piperazinyl]phenyl}-4-nitrobenzamide | 524 |
| 97 | 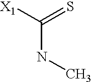 | N-(3H-Imidazol-4-ylmethyl)-N-[4- (4-methylthiocarbamoyl-1- piperazinyl)phenyl]-4-nitrobenzamide | 480 |
| 98 | 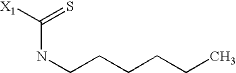 | N-[4-(4-Hexylthiocarbamoyl-1- piperazinyl)phenyl]-N-(3H-imidazol- 4-ylmethyl)-4-nitrobenzamide | 550 |
| 99 | 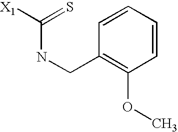 | N-(3H-Imidazol-4-ylmethyl)-N-{4- [4-(2-methoxybenzylthiocarbamoyl)- 1-piperazinyl]phenyl}-4-nitrobenzamide | 586 |
| 100 | 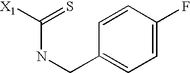 | N-{4-[4-(4-Fluorobenzylthio- carbamoyl)-1-piperazinyl]phenyl}- N-(3H-imidazol-4-ylmethyl)-4- nitrobenzamide | 574 |

### EXAMPLES 101 TO 105

### EXAMPLE 101A

### Cyclohexanecarboxylic acid (4-1-piperazinylphenyl)(1-trityl-1H-imidazol-4-ylmethyl)amide resin

Resin 101A is prepared from resin 2C (2.6 g; 1.03 mmol/g; 2.68 mmol) and from clohexanecarboxylic acid chloride (1.44 ml; 10.7 mmol) according to the conditions used for the preparation of resin 2E.

### EXAMPLES 101 to 105

Compounds 101 to 105 were synthesized using resin 101A (80 mg; 1.1 mmol/g; 0.096 mmol) and isothiocyanates (0.38 mmol) according to the conditions used for the preparation of Examples 40 to 47.

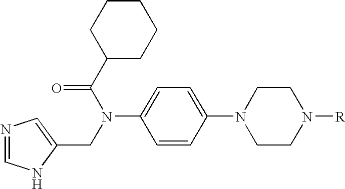

| | | | Mass spectrum |
|---|---|---|---|
| Example | R | Compound name | (M + H)+ |
| 101 | 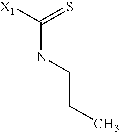 | Cyclohexanecarboxylic acid (3H- imidazol-4-ylmethyl)-[4-(4- propylthiocarbamoyl-1- piperazinyl)phenyl]amide | 469 |
| 102 | 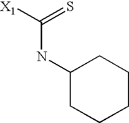 | Cyclohexanecarboxylic acid [4-(4- cyclohexylthiocarbamoyl-1- piperazinyl)phenyl]-(3H-imidazol-4- ylmethyl)amide | 509 |
| 103 | 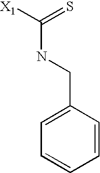 | Cyclohexanecarboxylic acid [4-(4- benzylthiocarbamoyl-1- piperazinyl)phenyl]-(3H-imidazol-4- ylmethyl)amide | 517 |
| 104 | 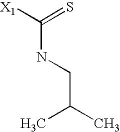 | Cyclohexanecarboxylic acid (3H- imidazol-4-ylmethyl)-[4-(4- isobutylthiocarbamoyl-1- piperazinyl)phenyl]amide | 483 |
| 105 | 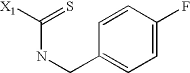 | Cyclohexanecarboxylic acid {4-[4-(4- fluorobenzylthiocarbamoyl)-1- piperazinyl]phenyl}(3H-imidazol-4- ylmethyl)amide | 535 |

### EXAMPLE 106

### 4-[5-({4-[4-(2-Thiophenecarbonyl)-1-piperazinyl]phenylamino}methyl)imidazol-1-ylmethyl]-benzonitrile

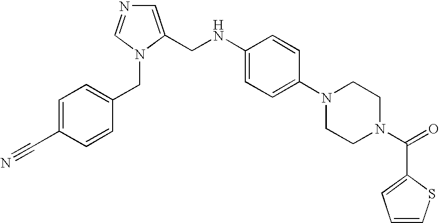

### EXAMPLE 106A

### 4-(5-Formylimidazol-1-ylmethyl)benzonitrile

1-Trityl-1H-imidazole-4-carboxaldehyde (Daninos-Zeghal S. et al., Tetrahedron, 1997, 53(22), 7605-14) (25 g; 74.0 mmol) dissolved in dichloromethane (125 ml) in the presence of sodium iodide (16.6 g; 111.0 mmol) is treated with 4-cyanobenzyl bromide (21.74 ml; 111.0 mmol) at room temperature. The medium is then refluxed, under nitrogen, for 24 hours and then diluted with dichloromethane and washed with saturated NaHCO₃ solution and with water. The organic phase is dried over sodium sulfate, filtered and then evaporated to dryness. The syrup obtained is purified by chromatography on a column of silica eluted with a 9/1 and then 1/1 CH₂Cl₂/acetone mixture to give the pure product in the form of a yellow solid (4.8 g; 27%).

¹H NMR, d₆-DMSO (ppm): 5.62 s, 2H; 7.32 d, 2H; 7.82 d, 2H; 8.01 s, 1H; 8.31 s, 1H; 9.70 s, 1H.

### EXAMPL 106

### 4-[5-({4-[4-(2-Thiophenecarbonyl)-1-piperazinyl]phenylamino}-methyl)imidazol-1-ylmethyl]benzonitrile

A mixture of compound 106A (50 mg; 0.24 mmol) and compound 1B (68 mg; 0.237 mmol) in 1,2-dichloroethane (1.2 ml) in the presence of acetic acid (74 μl; 1.42 mmol) is treated with sodium triacetoxyborohydride (55 mg; 0.26 mmol) at room temperature. After stirring overnight, the medium is diluted with ethyl acetate, washed with saturated NaHCO₃ solution, with water and then with saturated NaCl solution. The organic phase is dried over Na₂SO₄, filtered and evaporated to dryness. The syrup obtained is purified by chromatography on a column of silica eluted with a 97.75/2/0.25 CH₂Cl₂/MeOH/NH₄OH mixture to give the pure compound in the form of a beige-colored syrup (86 mg; 75%).

¹H NMR, d₆-DMSO (ppm): 2.95 t, 4H; 3.75 t, 4H; 3.99 d, 2H; 5.36 s, 2H; 5.47 t, 1H; 6.46 d, 2H; 6.74 d, 2H; 6.89 s, 1H; 7.13 dd, 1H; 7.24 d, 2H; 7.43 dd, 1H; 7.72 d, 1H; 7.75 dd, 1H; 7.80 d, 2H;

Mass spectrum (ESI): m/z 483 (MH⁺).

### EXAMPLE 107

### 4-[5-({4-[4-(5-Chloro-2-thiophenecarbonyl)-1-piperazinyl]phenylamino}methyl)-imidazol-1-ylmethyl]benzonitrile

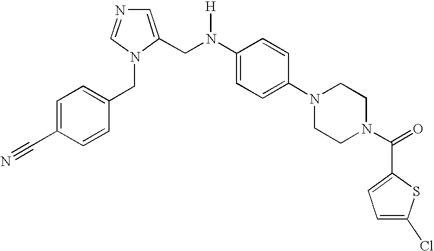

Compound 107 is prepared from compound 106A (2 g; 9.47 mmol) and compound 57A (3.05 g; 9.47 mmol) according to the conditions used for the preparation of Example 106. The product is obtained in the form of a beige-colored syrup (4.38 g; 89%).

¹H NMR, d₆-DMSO (ppm): 2.95 t, 4H; 3.74 broad s, 4H; 3.99 d, 25H; 5.36 s, 2H; 5.47 t, 1H; 6.46 d, 2H; 6.73 d, 2H; 6.88 s, 1H; 7.18 d, 1H; 7.24 d, 2H; 7.34 d, 1H; 7.72d, 1H; 7.80d, 2H;

Mass spectrum (ESI): m/z 517 (MH⁺).

### EXAMPLES 108 TO 124

Compounds 108 to 124 were synthesized according to the following general procedure:

Compound 107 (50 mg; 0.097 mmol) dissolved in dichloromethane (1.5 ml) in the presence of polystyrene-diisopropylethylamine (PS-DIEA) resin (80 mg; 3.67 mmol/g; 0.291 mmol) is treated at room temperature with an acid chloride (0.126 mmol) for 1 hour 20 minutes. The medium is then treated by addition of PS-trisamine resin (106 mg; 3.66 mmol/g; 0.39 mmol) and stirred at room temperature for 5 hours. The medium is filtered and the resins are rinsed with dichloromethane and methanol. The filtrate is evaporated to dryness to give the desired product.

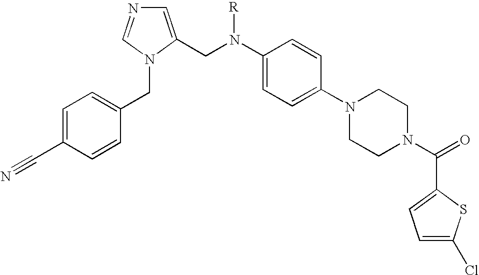

| | | | Mass spectrum |
|---|---|---|---|
| Example | R | Compound name | (M + H)+ |
| 108 | 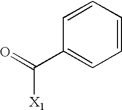 | N-{4-[4-(5-Chloro-2- thiophenecarbonyl)-1- piperazinyl]phenyl}N-[3-(4- cyanobenzyl)-3H-imidazol-4- ylmethyl]benzamide | 621 and 623 |
| 109 | 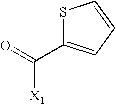 | Thiophene-2-carboxylic acid {4-[4-(5- chloro-2-thiophenecarbonyl)-1- piperazinyl]phenyl}[3-(4- cyanobenzyl)-3H-imidazol-4- ylmethyl]amide | 627 and 629 |
| 110 | 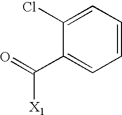 | 2-Chloro-N-{4-[4-(5-chloro-2- thiophenecarbonyl)-1- piperazinyl]phenylk}-N-[3-(4- cyanobenzyl)-3H-imidazol-4- ylmethyl]benzamide | 655 and 657 |
| 111 | 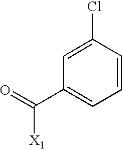 | 3-Chloro-N-{4-[4-(5-chloro-2- thiophenecarbonyl)-1- piperazinyl]phenyl}-N-[3-(4- cyanobenzyl)-3H-imidazol-4- ylmethyl]benzamide | 655 and 657 |
| 112 | 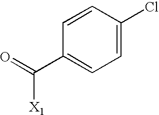 | 4-Chloro-N-{4-[4-(5-chloro-2- thiophenecarbonyl)-1- piperazinyl]phenyl}-N-[3-(4- cyanobenzyl)-3H-imidazol-4- ylmethyl]benzamide | 655 and 657 |
| 113 | 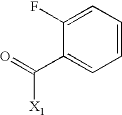 | N-{4-[4-(5-Chloro-2- thiophenecarbonyl)-1- piperazinyl]phenyl}-N-[3-(4- cyanobenzyl)-3H-imidazol-4- ylmethyl]-2-fluorobenzamide | 639 and 641 |
| 114 | 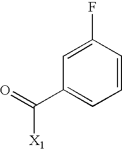 | N-{4-[4-(5-Chloro-2- thiophenecarbonyl)-1- piperazinyl]phenyl}-N-[3-(4- cyanobenzyl)-3H-imidazol-4- ylmethyl]-3-fluorobenzamide | 639 and 641 |
| 115 | 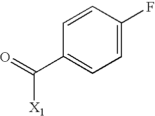 | N-{4-[4-(5-Chloro-2- thiophenecarbonyl)-1- piperazinyl]phenyl}-N-[3-(4- cyanobenzyl)-3H-imidazol-4- ylmethyl]-4-fluorobenzamide | 639 and 641 |
| 116 | 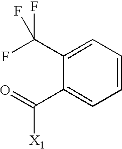 | N-{4-[4-(5-Chloro-2- thiophenecarbonyl)-1- piperazinyl]phenyl}-N-[3-(4- cyanobenzyl)-3H-imidazol-4- ylmethyl]-2-trifluoromethylbenzamide | 689 and 691 |
| 117 | 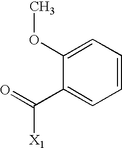 | N-{4-[4-(5-Chloro-2- thiophenecarbonyl)-1- piperazinyl]phenyl}-N-[3-(4- cyanobenzyl)-3H-imidazol-4- ylmethyl]-2-methoxybenzamide | 651 and 653 |
| 118 | 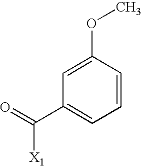 | N-{4-[4-(5-Chloro-2- thiophenecarbonyl)-1- piperazintl]phenyl}-N-[3-(4- cyanobenzyl)-3H-imidazol-4- ylmethyl]-3-methoxybenzamide | 651 and 653 |
| 119 | 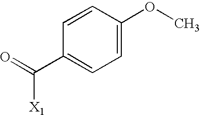 | N-{4-[4-(5-Chloro-2- thiophenecarbonyl)-1- piperazinyl]phenyl}-N-[3-(4- cyanobenzyl)-3H-imidazol-4- ylmethyl]-4-methoxybenzamide | 651 and 653 |
| 120 | 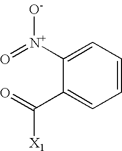 | N-{4-[4-(5-Chloro-2- thiophenecarbonyl)-1- piperazinyl]phenyl}-N-[3-(4- cyanobenzyl)-3H-imidazol-4- ylmethyl]-2-nitrobenzamide | 666 and 668 |
| 121 | 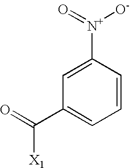 | N-{4-[4-(5-Chloro-2- thiophenecarbonyl)-1- piperazinyl]phenyl}-N-[3-(4- cyanobenzyl)-3H-imidazol-4- ylmethyl]-3-nitrobenzamide | 666 and 668 |
| 122 | 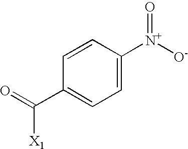 | N-{4-[4-(5-Chloro-2- thiophenecarbonyl)-1- piperazinyl]phenyl}-N-[3-(4- cyanobenzyl)-3H-imidazol-4- ylmethyl]-4-nitrobenzamide | 666 and 668 |
| 123 | 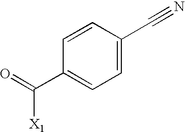 | N-{4-[4-(5-Chloro-2- thiophenecarbonyl)-1- piperazinyl]phenyl}-4-cyano-N-[3-(4- cyanobenzyl)-3H-imidazol-4- ylmethyl]-benzamide | 646 and 648 |
| 124 | 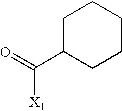 | Cyclohexanecarboxylic acid {4-[4-(5- chloro-2-thiophenecarbonyl)-1- piperazinyl]phenyl}-[3-(4- cyanobenzyl)-3H-imidazol-4- ylmethyl]amide | 627 and 629 |

### EXAMPLES 125 TO 128

Compounds 125 to 128 were synthesized according to the following general procedure:

Compound 107 (50 mg; 0.097 mmol) dissolved in dichloromethane (1.5 ml) in the presence of PS-DIEA resin (80 mg; 3.67 mmol/g; 0.291 mmol) is treated at room temperature with sulfonyl chloride (0.126 mmol) for 1 hour 20 minutes. The medium is then treated by addition of PS-trisamine resin (106 mg; 3.66 mmol/g; 0.39 mmol) and stirred at room temperature for 5 hours. The medium is filtered and the resins are rinsed with dichloromethane and methanol. The filtrate is evaporated to dryness to give the desired product.

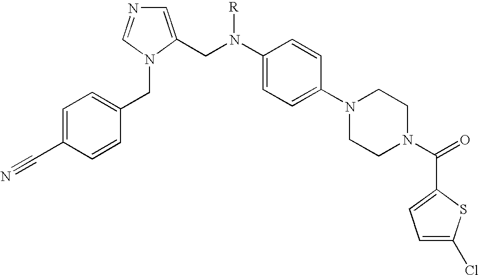

| | | | Mass spectrum |
|---|---|---|---|
| Example | R | Compound name | (M + H)+ |
| 125 | 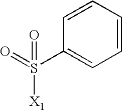 | N-{4-[4-(5-Chloro-2- thiophenecarbonyl)-1- piperazinyl]phenyl}-N-[3-(4- cyanobenzyl)-3H-imidazol-4- ylmethyl]benzenesulfonamide | 657 and 659 |
| 126 | 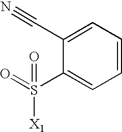 | N-{4-[4-(5-Chloro-2- thiophenecarbonyl)-1- piperazinyl]phenyl}-2-cyano-N-[3-(4- cyanobenzyl)-3H-imidazol-4- ylmethyl]benzenesulfonamide | 682 and 684 |
| 127 | 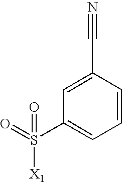 | N-{4-[4-(5-Chloro-2- thiophenecarbonyl)-1- piperazinyl]phenyl}-3-cyano-N-[3-(4- cyanobenzyl)-3H-imidazol-4- ylmethyl]benzenesulfonamide | 682 and 684 |
| 128 | 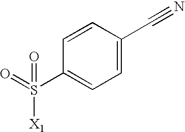 | N-{4-[4-(5-Chloro-2- thiophenecarbonyl)-1- piperazinyl]phenyl}-4-cyano-N-[3-(4- cyanobenzyl)-3H-imidazol-4- ylmethyl]benzenesulfonamide | 682 and 684 |

### EXAMPLE 129

### 3-(4-Cyanobenzyl)-3H-imidazole-4-carboxylic acid {4-[4-(5-chloro-2-thiophenecarbonyl)-1-piperazinyl]phenyl}amide

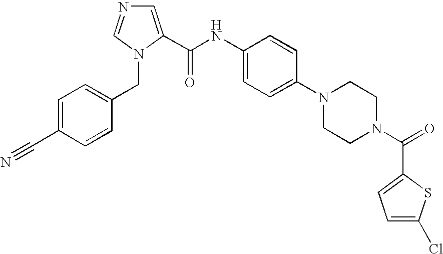

### EXAMPLE 129A

### 1-Trityl-1H-imidazole-4-carboxylic acid {4-[4-(5-chloro-2-thiophenecarbonyl)-1-piperazinyl]phenyl}amide

A mixture of 1-trityl-1H-imidazole-4-carboxylic acid (Hunt, J. J. et al. J. Med. Chem., 1996, 39/2, 353-358) (373 mg; 1.05 mmol) and of compound 57A (308 mg; 0.956 mmol) dissolved in dichloromethane (4 ml) in the presence of DIEA (0.25 ml; 1.43 mmol) is treated with HOOBt (171 mg; 1.05 mmol) and EDC (202 mg; 1.05 mmol) at room temperature for 4 hours. The medium is taken up in ethyl acetate and washed with water. The organic phase is dried over sodium sulfate, filtered and evaporated to dryness. The syrup obtained is purified by chromatography on a column of silica eluted with a 97.75/2/0.25 CH₂Cl₂/MeOH/NH₄OH mixture to give the pure compound (444 mg; 70%).

Mass spectrum (ESI): m/z 658 (MH⁺).

### EXAMPLE 129

### 3-(4-Cyanobenzyl)-3H-imidazole-4-carboxylic acid {4-[4-(5-chloro-2-thiophenecarbonyl)-1-piperazinyl]phenyl}amide

Compound 129A (222 mg; 0.337 mmol) dissolved in ethyl acetate (2.9 ml) is treated with 4-cyanobenzyl bromide (69 mg; 0.353 mmol). The mixture is stirred at reflux for 16 hours, potassium iodide (56 mg; 0.337 mmol) is added and further 4-cyanobenzyl bromide (69 mg; 0.353 mmol) is added, and the mixture is then stirred for a further 12 hours at reflux. The medium is taken up in methanol (2 ml) and then stirred at reflux for 1 hour 30 minutes. The mixture is evaporated to dryness and the syrup obtained is purified by chromatography on a column of silica eluted with a 97.75/2/0.25 Cl₂/MeOH/NH₄OH mixture to give the pure product (18 mg; 11%).

¹H NMR, d₆-DMSO (ppm): 3.15 t, 4H; 3.77 broad s, 4H; 5.67 s, 2H; 6.92 d, 2H; 7.18 d, 1H; 7.30 d, 2H; 7.37 d, 1H; 7.47 d, 2H; 7.79 d, 2H; 7.84 s, 1H; 8.07 s, 1H; 9.95 s, 1H;

Mass spectrum (ESI): m/z 531 and 533 (MH⁺).

### EXAMPLE 130

### N-{4-[4-(5-Chloro-2-thiophenecarbonyl)-1-piperazinyl]phenyl}-3-[3-(4-cyanobenzyl)-3H-imidazol-4-yl]propionamide

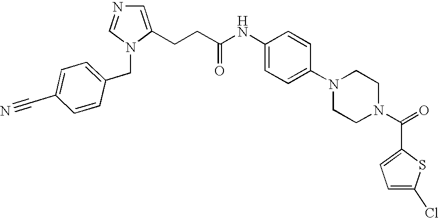

Compound 130 is prepared from 3-(1-trityl-1H-imidazol-4-yl)propionic acid (402 mg; 1.05 mmol) and compound 57A (308 mg; 0.956 mmol) according to the conditions described for the preparation of Example 129. The product is isolated pure in the form of a beige-colored solid (18 mg; 10%).

¹H NMR, d₆-DMSO (ppm): 2.53 t, 2H; 2.64 t, 2H; 3.13 t, 4H; 3.77 broad s, 45H; 5.33 s, 2H; 6.74 s, 1H; 6.89 d, 2H; 7.18 d, 1H; 7.24 d, 2H; 7.38 d, 1H; 7.42 d, 2H; 7.74 s, 1H; 7.84 d, 2H; 9.77s, 1H;

Mass spectrum (ESI): m/z 559 (MH⁺).

### EXAMPLE 131

### N-{4-[4-(5-Chloro-2-thiophenecarbonyl)-1-piperazinyl]phenyl}-3-(3H-imidazol-4-yl)acrylamide

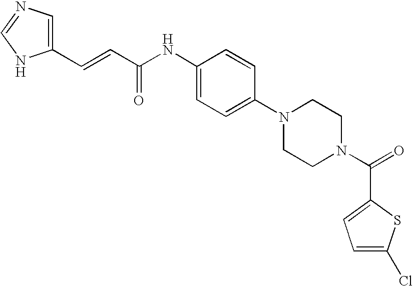

During the reaction for the formation of compound 130, a side product corresponding to the structure of compound 131 is formed, which is isolated during the purification (30 mg; 16%) in the form of a beige-colored syrup.

¹H NMR, d₆-DMSO (ppm): 2.56 t, 2H; 2.76 t, 2H; 3.13 t, 4H; 3.77 broad s, 4H; 6.81 s, 1H; 6.90 d, 2H; 7.17 d, 2H; 7.37 d, 2H; 7.46 d, 2H. 7.51 s, 1H; 9.77s, 1H;

Mass spectrum (ESI): m/z 444 (MH⁺).

### EXAMPLE 132

### N-{4-[4-(5-Chloro-2-thiophenecarbonyl)-1-piperazinyl]phenyl}-3-(3H-imidazol-4-yl)acrylamide

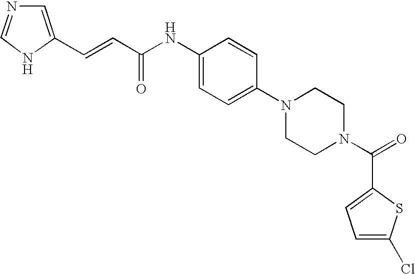

Compound 132 is obtained under the same conditions as for compound 131, except that in this specific case it is the only compound formed during the reaction. It is formed from 3-(1-trityl-1H-imidazol-4-yl)acrylic acid (400 mg; 1.05 mmol) and compound 57A (308 mg; 0.956 mmol) to give the pure product in the form of a yellow solid (70 mg, 33%).

¹H NMR, d₆-DMSO (ppm): 3.15 t, 4H; 3.78 broad s, 3H; 6.70 d, 1H; 6.92 d, 2H; 7.17 d, 1H; 7.37 d, 1H; 7.39 d, 1H; 7.43 s, 1h. 7.57 d, 2H; 7.74 s, 1H; 9.92 s, 1H; 12.36 broad s, 1H;

Mass spectrum (ESI): m/z 442 (MH⁺).

### EXAMPLE 133

### 2-[3-(4-Cyanobenzyl)-3H-imidazol-4-yl]-N-{4-[4-(thiophene-2-carbonyl)-1-piperazinyl]phenyl}acetamide

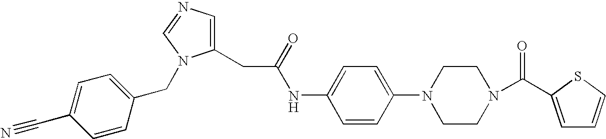

Compound 133 is prepared from [3-(4-cyanobenzyl)-3H-imidazol-4-yl]acetic acid (70 mg; 0.290 mmol) and compound 1B (110 mg; 0.319 mmol) according to the conditions described for the preparation of example 129A.

Mass spectrum (ESI): m/z 511 (MH⁺).

### EXAMPLE 134

### (5-Chloro-2-thiophenyl)-{4-[4-(cyclohexylmethylpyridin-3-ylmethylamino)phenyl]-1-piperazinyl}methanone

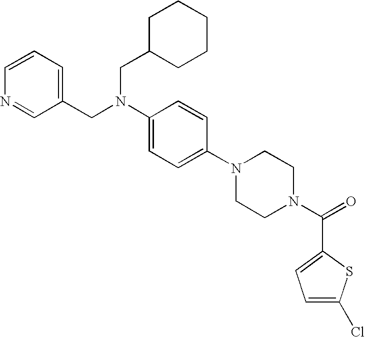

### EXAMPLE 134A

### (5-Chloro-2-thiophenyl)-(4-{4-[(pyridin-3-ylmethyl)amino]phenyl}-1-piperazinyl]methanone

Compound 134A is prepared from 3-pyridinecarboxaldehyde (88 μl; 0.934 mmol) according to the conditions described for the preparation of Example 106. The product is isolated pure in the form of a yellow syrup (366 mg; 95%).

¹H NMR, d₆-DMSO (ppm): 2.94 t, 4H; 3.74 broad s, 4H; 4.24 d, 2H; 5.90 t, 1H; 6.53 d, 2H; 6.75 d, 2H; 7.16 d, 1H; 7.33 m, 2H; 7.73 d, 1H; 8.41 dd, 1H; 8.55 d, 1H;

Mass spectrum (ESI): m/z 4/3 (MH⁺).

### EXAMPLE 135

### {4-[4-(Benzyl-3-pyridylmethylamino)phenyl]-1-piperazinyl}-(5-chloro-2-thiophenyl)methanone

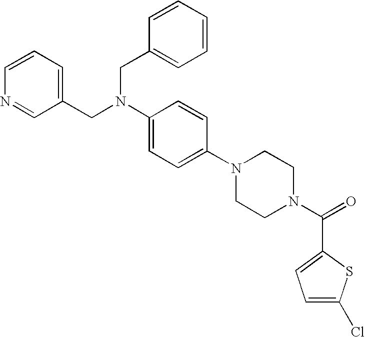

Compound 134A (45 mg; 0.109 mmol) dissolved in DMSO (0.5 ml) is treated, at room temperature, with benzyl bromide (14 μl; 0.120 mmol) and then, 15 minutes later, with 1,8-diazabicyclo[5.4.0]undec-7-ene (DBU) (25 μl; 0.163 mmol). The mixture is stirred overnight at room temperature and is then diluted with ethyl acetate and washed with water and then with saturated NaC1 solution. The organic phase is dried over sodium sulfate, filtered and evaporated to dryness. The crude product is purifed by chromatography on a column eluted with a 5/1 CH₂Cl₂/acetone mixture +0.2% Et₃N. The pure product is isolated in the form of a yellow syrup (28 mg; 51%).

Mass spectrum (ESI): m/z 503 (MH⁺).

### EXAMPLES 136 TO 140

Compounds 136 to 140 were synthesized from compound 134A (45 mg; 0.105 mmol) according to the conditions described for the preparation of Examples 108 to 124 and 125 to 128.

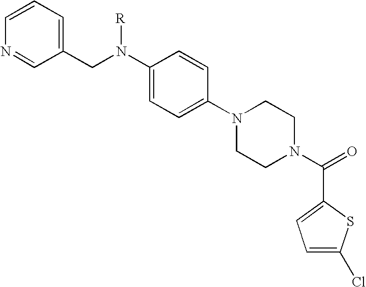

| | | | Mass spectrum |
|---|---|---|---|
| Example | R | Compound name | (M + H)⁺ |
| 136 | 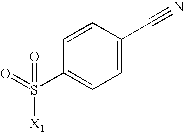 | N-{4-[4-(5-Chloro-2- thiophenecarbonyl)-1- piperazinyl]phenyl}-4-cyano-N-3- pyridylmethylbenzenesulfonamide | 578 and 580 |
| 137 | 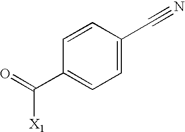 | N-{4-[4-(5-Chloro-2- thiophenecarbonyl)-1- piperazinyl]phenyl}-4-cyano-N-3- pyridylmethyl-benzamide | 542 and 544 |
| 138 | 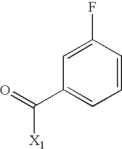 | N-{4-[4-(5-Chloro-2- thiophenylcarbonyl)-1- piperazinyl]phenyl}-3-fluoro-N-3- pyridylmethylbenzamide | 535 and 537 |
| 139 | 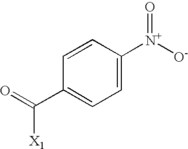 | N-{4-[4-(5-Chloro-2- thiophenecarbonyl)-1- piperazinyl]phenyl}-4-nitro-N-3- pyridylmethylbenzamide | 562 and 564 |
| 140 | 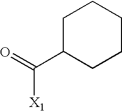 | Cyclohexanecarboxylic acid {4-[4-(5- chloro-2-thiophenecarbonyl)-1- piperazinyl]phenyl}-3- pyridylmethylamide | 523 and 525 |

### EXAMPLE 141

### 4-{4-[Benzenesulfonyl-(3H-imidazol-4-ylmethyl)amino]phenyl}piperidine-1-carbothioic acid isobutylamide

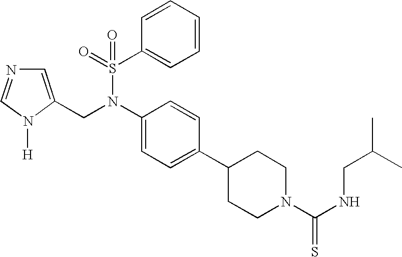

### EXAMPLE 141A

### 4-(4-Nitrophenyl)piperidine

4-Phenyl-1,2,3,6-tetrahydropyridine hydrochloride (10.38 g; 53.0 mmol) dissolved in methanol (160 ml) in the presence of palladium-on-charcoal (5%) (1.04 g) is hydrogenated under 30 psi of hydrogen for 1 hour. The medium is filtered through Celite and the filtrate is evaporated to dryness to give 4-phenylpiperidine hydrochloride (10.17 g; 97%). This intermediate (13 g; 65.7 mmol) is taken up in chloroform (430 ml), cooled to 0° C. and then treated with copper nitrate 2.5-hydrate (15.3 g; 65.7 mmol) for 15 minutes. The mixture is then added dropwise over 45 minutes to a solution of trifluoroacetic anhydride (65 ml; 460 mmol) in chloroform (70 ml). After stirring at 0° C. for 48 hours, the medium is poured onto ice, diluted with dichloromethane and neutralized by addition of concentrated sodium hydroxide (85 ml). The medium is extracted several times with dichloromethane and the organic phases are then combined, dried over magnesium sulfate, filtered and evaporated to dryness. The syrup obtained is purified by chromatography on a column of silica eluted with a 96.5/3/0.5 CH₂Cl₂/MeOH/NH₄OH mixture to give pure compound 141A (8.35 g; 68%).

¹H NMR, d₆-DMSO (ppm): 1.52 m, 2H; 2.59 t, 2H; 2.75 dt, 1H; 3.19 broad s, 2H; 7.52 d, 2H; 8.16 d, 2H;

Mass spectrum (ESI): 207 (MH⁺).

### EXAMPLE 141B

### 4-(4-Nitrophenyl)piperidine-1-carboxylic acid 9H-fluoren-9-ylmethyl ester

Compound 141A (500 mg; 2.42 mmol) dissolved in DMF (18 ml) is treated at 0° C. with Fmoc-succinimide (981 mg; 2.91 mmol) dissolved in DMF (10 ml). After stirring for 30 minutes at 0° C., the medium is diluted with dichloromethane and washed with water and with HCl solution (1 N). The organic phase is dried over MgSO₄, filtered and evaporated to dryness. The syrup obtained is purifed by chromatography on a column of silica eluted with an 85/15 to 70/30 petroleum ether/ethyl acetate mixture. The pure product is obtained in the form of a pale yellow syrup (834 mg; 80%).

¹H NMR, d₆-DMSO (ppm): 1.30 m, 2H; 1.70 broad s, 2H; 2.85 m, 3H; 3.88 broad s, 1H; 4.07 broad s, 1H; 4.29 t, 1H; 4.40 broad s, 1H; 4.50 broad s, 1H; 7.35 t, 2H; 7.42 t, 2H; 7.50 d, 2H; 7.65 d, 2H; 7.86 d, 2H; 8.18 d, 2H.

Mass spectrum (ESI): m/z 429 (MH⁺).

### EXAMPLE 141C

### 4-(4-Aminophenyl)piperidine-1-carboxylic acid 9H-fluoren-9-ylmethyl ester

Compound 141B (5.02 g; 11.7 mmol) dissolved in an MeOH/THF (4/1) mixture in the presence of HCl (2 N) (2.9 ml; 58.5 mmol) and a catalytic amount of palladiumon-charcoal (at 5%) (251 mg) is hydrogenated under an atmospheric pressure of hydrogen for 6 hours. The medium is then filtered through Celite and the filtrate is evaporated to dryness. The syrup obtained is taken up in CH₂Cl₂ and washed with sodium hydroxide (1N) and then with water. The organic phase is dried over MgSO₄, filtered and evaporated to dryness. The syrup obtained is chromatographed on a column of silica eluted with a 9/1 petroleum ether/ethyl acetate mixture. The pure product is obtained in the form of a white solid (4.29 g; 85%).

¹H NMR, d₆-DMSO (ppm): 1.25 broad s, 2H; 1.60 broad s, 2H; 2.78 broad s, 3H; 3.87 broad s, 1H; 4.02 d, 1H; 4.28 t, 1H; 4.36 broad s, 1H; 4.45 broad s, 1H; 4.85 s, 2H; 6.49 d, 2H; 6.83 d, 2H; 7.34 t, 2H; 7.41 t, 2H; 7.64 d, 2H; 7.89 d, 2H.

Mass spectrum (ESI): m/z 399 (MH⁺)

| | | | | |
|---|---|---|---|---|
| Elemental analysis | % calculated: | C 78.36; | H 6.58; | N 7.03 |
| (C₂₆H₂₆N₂O₂) | % found: | C 77.98; | H 6.61; | N 7.05 |

### EXAMPLE 141D

### 4-{4-[(1-Trityl-1H-imidazol-4-ylmethyl)aminophenyl)-piperidine-1-carboxylic acid 9H-fluoren-9-ylmethyl ester resin

Resin 141D is prepared from resin 1C (2.0 g; 1.7 mmol/g; 3.4 mmol) and compound 141C (_{2.44} g; 6.12 mmol) according to the conditions used for the preparation of Example 1D. The resin obtained (3.25 g) is monitored by HPLC analysis of a sample after cleavage (1/4 TFA/CH₂Cl₂) and has a purity of 91% (HPLC C18, 220 nm, 100% H₂O to 100% CH₃CN (+0.1% TFA) over 10 min).

### EXAMPLE 141E

### N-(4-Piperidin-1-yl-phenyl)-N-(1-trityl-1H-imidazol-4-ylmethyl)benzenesulfonamide resin

Resin 141E is prepared from resin 141D (506.9 mg; 1.03 mmol/g; 0.522 mmol) according to the conditions used for the preparation of Example 2E starting with 2C.

### EXAMPLE 141

### 4-{4-[Benzenesulfonyl-(3H-imidazol-4-ylmethyl)amino]phenyl}-piperidine-1-carbothioic acid isobutylamide

Compound 141 is prepared from resin 141E according to the conditions used for the preparation of Examples 40 to 47.

Mass spectrum (ESI): m/z 512 (M+H⁺).

### EXAMPLES 142 TO 157

Compounds 142 to 157 were prepared from resin 141E according to the conditions used for the preparation of Example 141.

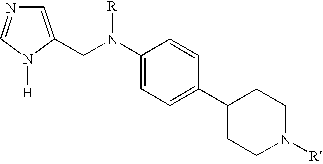

| | | | | Mass |
|---|---|---|---|---|
| | | | | spectrum |
| Example | R | R′ | Compound name | (M + H⁺) |
| 142 | 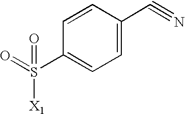 | 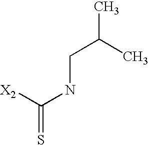 | 4-{4-[(4-Cyano- benzenesulfonyl)-(3H- imidazol-4- ylmethyl)amino]phenyl}piperidine-1-carbothioic acid isobutylamide | 537 |
| 143 | 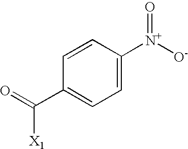 | 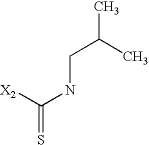 | N-(3H-Imidazol-4- ylmethyl)-N-[4-(1- isobutylthiocarbamoyl- piperidin-4-yl)phenyl]-4- nitrobenzamide | 521 |
| 144 | 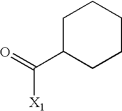 | 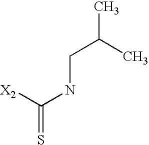 | Cyclohexanecarboxylic acid (3H-imidazol-4- ylmethyl)-[4-(1- isobutylthiocarbamoyl- piperidin-4-yl)phenyl]- amide | 482 |
| 145 | 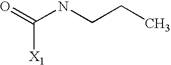 | 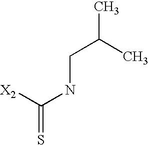 | 4-{4-[1-(3H-Imidazol-4- ylmethyl)-3-propyl- ureido]phenyl}- piperidine-1-carbothioic acid isobutylamide | 457 |
| 146 | 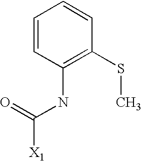 | 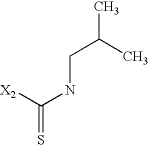 | 4-{4-[1-(3H-Imidazol-4- ylmethyl)-3-(2- methylsulfanylphenyl) ureido]phenyl}- piperidin-1-carbothioic acid isobutylamide | 537 |
| 147 | 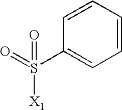 | 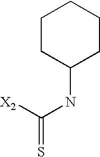 | 4-{4-[Benzenesulfonyl- (3H-imidazol-4- ylmethyl)amino]phenyl}piperidine-1-carbothioic acid cyclohexylamide | 538 |
| 148 | 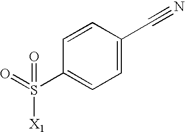 | 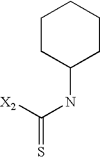 | 4-{4-[(4-Cyanobenzene- sulfonyl)-(3H-imidazol- 4-ylmethyl)amino]- phenyl}piperidine-1- carbothioic acid cyclohexylamide | 563 |
| 149 | 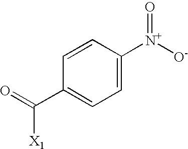 | 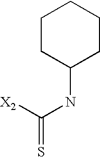 | N-[4-(1-Cyclohexyl- thiocarbamoylpiperidin- 4-yl)-phenyl]-N-(3H- imidazol-4-ylmethyl)-4- nitrobenzamide | 547 |
| 150 | 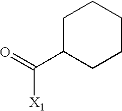 | 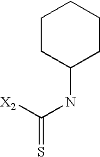 | Cyclohexanecarboxylic acid [4-(1-cyclohexyl- thiocarbamoylpiperidin- 4-yl)-phenyl]-(3H- imidazol-4-ylmethyl)- amide | 508 |
| 151 | 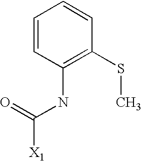 | 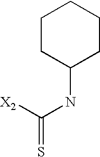 | 4-{4-[1-(3H-Imidazol-4- ylmethyl)-3-(2- methylsulfanylphenyl)- ureido]phenyl}- piperidine-1-carbothioic acid cyclohexylamide | 563 |
| 152 | 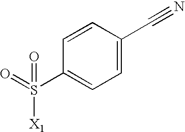 | 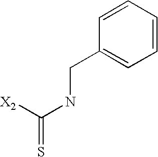 | 4-{4-[(4-Cyano-benzene- sulfonyl)(3H-imidazol-4- ylmethyl)amino]phenyl}- piperidine-1-carbothioic acid benzylamide | 571 |
| 153 | 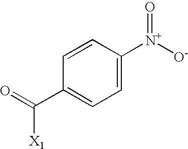 | 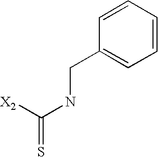 | N-[4-(1- Benzylthiocarbamoyl- piperidin-4-yl)phenyl]-N- (3H-imidazol-4- ylmethyl)-4- nitrobenzamide | 555 |
| 154 | 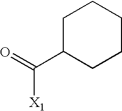 | 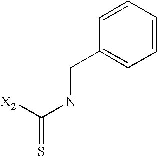 | Cyclohexanecarboxylic acid [4-(1- benzylthiocarbamoyl- piperidin-4-yl)-phenyl]- (3H-imidazol-4- ylmethyl)amide | 516 |
| 155 | 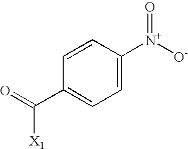 | 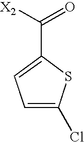 | N-{4-[1-(5-Chloro-2- thiophenecarbonyl)- piperidin-4-yl]phenyl}- N-(3H-imidazol-4- ylmethyl)-4- nitrobenzamide | 550 |
| 156 | 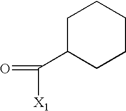 | 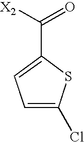 | Cyclohexanecarboxylic acid {4-[1-(5-chloro-2- thiophenecarbonyl)- piperidin-4-yl]phenyl}- (3H-imidazol-4- ylmethyl)amide | 511 |
| 157 | 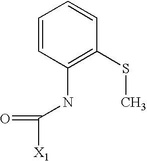 | 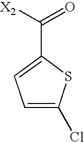 | 1-{4-[1-(5-Chloro-2- thiophenecarbonyl)- piperidin-4-yl]phenyl}-1- (3H-imidazol-4- ylmethyl)-3-(2- methylsulfanylphenyl)- urea | 566 |

### EXAMPLE 158

### 4-{4-[(3H-Imidazol-4-ylmethyl)amino]phenyl}-1-piperazinecarbothioic acid isobutylamide

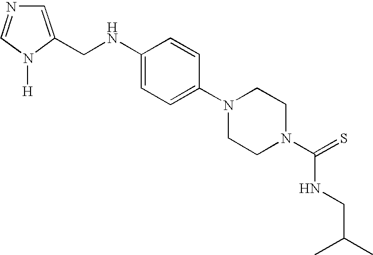

### EXAMPLE 158A

### 4-(4-Nitrophenyl)-1-piperazinecarbothioic acid isobutylamide

1-(4-Nitrophenyl)piperazine (10 g; 48.2 mmol) in toluene (200 ml) is treated at room temperature with isobutyl isocyanate (6.5 ml, 72.3 mmol). After stirring for 2 hours, the mixture is taken up in EtOAc and the organic phase is washed with water (2×) and then dried over Na₂SO₄ and evaporated to dryness. The product is obtained in the form of a yellow powder (15.5 g; 100%), which is used directly in the following reaction.

Mass spectrum (ESI): m/z 323 (MH⁺).

### EXAMPLE 158B

### 4-(4-Aminophenyl)-1-piperazinecarbothioic acid isobutylamide

Intermediate 158A (14.5 g; 45.1 mmol) dissolved in EtOH (300 ml) is treated with tin chloride dihydrate (51 g; _{225.5} mmol) at 72° C. for 19 hours. The mixture is poured onto ice (500 ml), EtOAc (500 ml) is then added and the medium is neutralized by addition of saturated NaHCO₃. The medium is extracted 3 times with EtOAc and the organic phase is then washed with water and with saturated NaCl solution, dried over Na₂SO₄ and evaporated to dryness. The product is obtained in the form of a yellow powder purified on a column of silica eluted with a 2/1 dichloromethane/acetone mixture and then a 97.75/2/0.25 CH₂Cl₂/MeOH/NH₄OH mixture to give the pure product in the form of a yellow powder (9.2 g; 69%).

Mass spectrum (ESI): m/z 293 (MH⁺).

### EXAMPL 158C

### 4-{4-[(1-Trityl-1H-imidazol-4-ylmethyl)amino]phenyl}-1-piperazinecarbothioic acid isobutylamide resin

Resin 158C is prepared from resin 1C (1 g; 1.94 mmol) and intermediate 158B according to the procedure described for the preparation of Example 1D.

### EXAMPLE 158

### 4-{4-[(3H-Imidazol-4-ylmethyl)amino]phenyl}-1-piperazinecarbothioic acid isobutylamide

A fraction of resin 158C (100 mg) is cleaved according to the procedure described for the prepration of Example 1, using 1D, to give compound 158 in the form of a colorless syrup (57 mg).

HPLC (C18, λ 220 nM, 100% H₂O to 100% CH₂CN (+0.1% TFA) over 8 min): purity 96%;

Mass spectrum (ESI): m/z 373 (MH⁺).

### EXAMPLE 59

### 4-{4-[(3H-Imidazol-4-ylmethyl)amino]phenyl}-1-piperazinecarbothioic acid cyclohexylamide

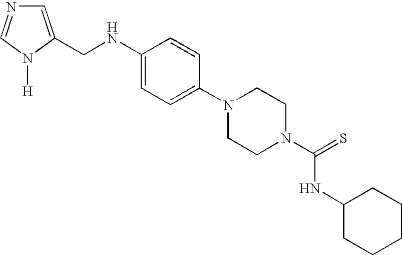

### EXAMPLE 159A

### 4-{4-[(1-Trityl-1H-imidazol-4-ylmethyl)amino]phenyl}-1-piperazinecarbothioic acid cyclohexylamide resin

Resin 159A is prepared from resin 1C (1 g; 1.94 mmol) according to the procedure described for the preparation of Example 158C.

### EXAMPLE 159

### 4-{4-[(3H-Imidazol-4-ylmethyl)amino]phenyl}-1-piperazinecarbothioic acid cyclohexylamide

A fraction of resin 159A (100 mg) is cleaved according to the procedure described for the preparation of Example 1, starting with 1D, to give compound 159 in the form of a colorless syrup (56 mg).

HPLC (C18, λ 220 nM, 100% H₂O to 100% CH₂CN (+0.1% TFA) over 8 mn): purity 88%;

Mass spectrum (ESI): m/z 399 (MH⁺).

### EXAMPLES 160 TO 176

Examples 160 to 176 are prepared from resin 158C or 159A (50 mg; 0.8 mmol/g; 0.040 mmol) according to the procedure described for the preparation of Examples 48 to 56.

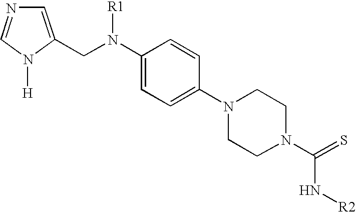

| | | | | Mass |
|---|---|---|---|---|
| | | | | spectrum |
| Example | R1 | R2 | Compound name | (M + H)+ |
| 160 | 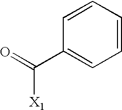 | 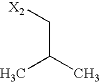 | N-(3H-Imidazol-4-ylmethyl)- N-[4-(4- isobutylthiocarbamoyl-1- piperazinyl)phenyl]- benzamide | 477 |
| 161 | 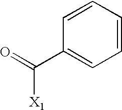 | 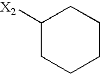 | N-[4-(4- Cyclohexylthiocarbamoyl-1- piperazinyl)phenyl]-N-(3H- imidazol-4-ylmethyl)- benzamide | 503 |
| 162 | 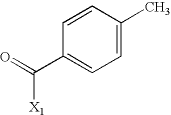 | 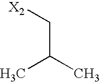 | N-(3H-Imidazol-4-ylmethyl)- N-[4-(4- isobutylthiocarbamoyl-1- piperazinyl)phenyl]-4- methylbenzamide | 491 |
| 163 | 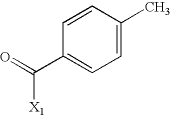 | 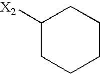 | N-[4-(4- Cyclohexylthiocarbamoyl-1- piperazinyl)phenyl]-N-(3H- imidazol-4-ylmethyl)-4- methylbenzamide | 517 |
| 164 | 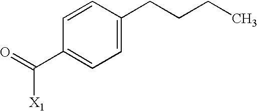 | 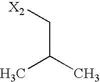 | 4-Butyl-N-(3H-imidazol-4- ylmethyl)-N-[4-(4- isobutylthiocarbamoyl-1- piperazinyl)phenyl]- benzamide | 533 |
| 165 | 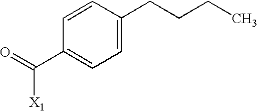 | 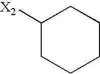 | 4-Butyl-N-[4-(4- cyclohexylthiocarbamoyl-1- piperazinyl)phenyl]-N-(3H- imidazol-4-ylmethyl)- benzamide | 559 |
| 166 | 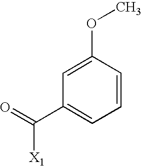 | 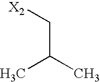 | N-(3H-Imidazol-4-ylmethyl)- N-[4-(4- isobutylthiocarbamoyl-1- piperazinyl)phenyl]-3- methoxybenzamide | 507 |
| 167 | 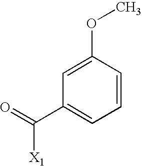 | 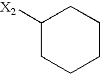 | N-[4-(4- Cyclohexylthiocarbamoyl-1- piperazinyl)phenyl]-N-(3H- imidazol-4-ylmethyl)-3- methoxybenzamide | 533 |
| 168 | 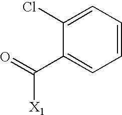 | 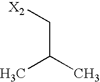 | 2-Chloro-N-(3H-imidazol-4- ylmethyl)-N-[4-(4- isobutylthiocarbamoyl-1- piperazinyl)phenyl]- benzamide | 511 |
| 169 | 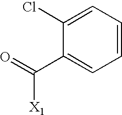 | 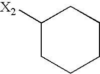 | 2-Chloro-N-[4-(4- cyclohexylthiocarbamoyl-1- piperazinyl)phenyl]-N-(3H- imidazol-4-ylmethyl)- benzamide | 537 |
| 170 | 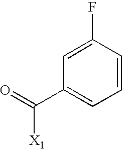 | 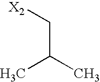 | 3-Fluoro-N-(3H-imidazol-4- ylmethyl)-N-[4-(4- isobutylthiocarbamoyl-1- piperazinyl)phenyl]- benzamide | 495 |
| 171 | 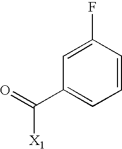 | 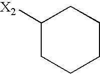 | N-[4-(4- Cyclohexylthiocarbamoyl-1- piperazinyl)phenyl]-3-fluoro- N-(3H-imidazol-4-ylmethyl)- benzamide | 521 |
| 172 | 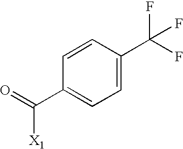 | 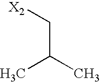 | N-(3H-Imidazol-4-ylmethyl)- N-[4-(4- isobutylthiocarbamoyl-1- piperazinyl)phenyl]-4- trifluoromethylbenzamide | 545 |
| 173 | 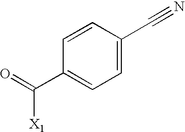 | 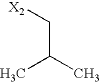 | 4-Cyano-N-(3H-imidazol-4- ylmethyl)-N-[4-(4- isobutylthiocarbamoyl-1- piperazinyl)phenyl]- benzamide | 502 |
| 174 | 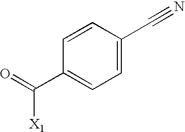 | 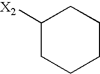 | 4-Cyano-N-[4-(4- cyclohexylthiocarbamoyl-1- piperazinyl)phenyl]-N-(3H- imidazol-4-ylmethyl)- benzamide | 528 |
| 175 | 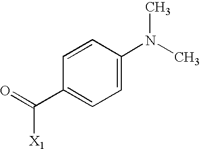 | 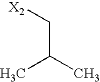 | 4-Dimethylamino-N-(3H- imidazol-4-ylmethyl)-N-[4- (4-isobutylthiocarbamoyl-1- piperazinyl)phenyl]- benzamide | 520 |
| 176 | 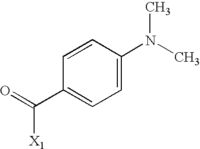 | 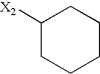 | N-[4-(4- Cyclohexylthiocarbamoyl-1- piperazinyl)phenyl]-4- dimethylamino-N-(3H- imidazol-4-ylmethyl)- benzamide | 546 |

### EXAMPLES 177 to 181

Examples 177 to 181 are prepared from resin 158C or 159A (75 mg; 1.1 mmol/g; 0.083 mmol) according to the procedure described for the preparation of Examples 48 to 56.

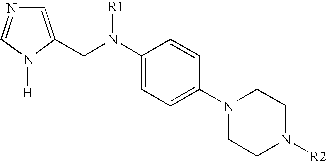

| | | | | Mass |
|---|---|---|---|---|
| | | | | spectrum |
| Example | R1 | R2 | Compound name | (M + H)+ |
| 177 | 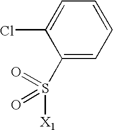 | 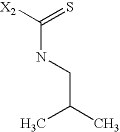 | 4-{4-[(2-Chloro- benzenesulfonyl)-(3H- imidazol-4- ylmethyl)amino]phenyl}-1- piperazinecarbothioic acid isobutylamide | 547 |
| 178 | 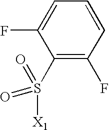 | 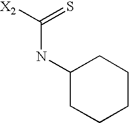 | 4-{4-[(2,6-Difluoro- benzenesulfonyl)(3H-imid- azol-4-ylmethyl)amino]- phenyl}-1- piperazinecarbothioic acid | 575 |
| 179 | 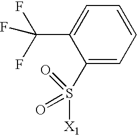 | 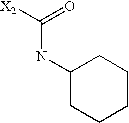 | 4-{4-[(3H-Imidazol-4- ylmethyl)-(2-trifluoro methylbenzenesulfonyl)- amino]phenyl}piperazine-1- carboxylic acid cyclohexylamide | 591 |
| 180 | 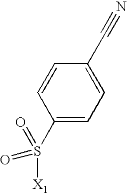 | 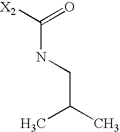 | 4-{4-[(4-Cyano- benzenesulfonyl)-(3H- imidazol-4- ylmethyl)amino]phenyl}- piperazine-1-carboxylic acid isobutylamide | 522 |
| 181 | 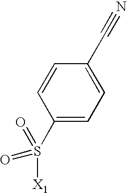 | 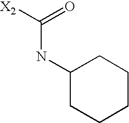 | 4-{4-[(4-Cyano- benzenesulfonyl)-(3H- imidazol-4- ylmethyl)amino]phenyl}- piperazine-1-carboxylic acid cyclohexylamide | 548 |

Following the acidic treatment during the cleavage, a conversion of certain thioureas into ureas is observed (Examples 179, 180 and 181).

### EXAMPLES 182 TO 186

Examples 182 to 186 are prepared according to the following general procedure: Resins 158C (100 mg; 0.8 mmolug; 0.1 mmol) or 159A (130 mg; 0.76 mmol/g; 0.1 mmol) are swollen with dichloromethane (3 ml) and are then treated with an aldehyde (0.5 mmol) at room temperature, in the presence of AcOH (58 μl, 1 mmol) and NaBH(OAc)₃ (0.51 mmol) for 24 hours. The resins are then filtered off and washed successively with DMF (3×); MeOH (1×); CH₂Cl₂ (1×), MeOH (1×), H₂O (2×) and MeOH (2×). The resins obtained are cleaved by treatment with a 5/5/1 TFA/CH₂Cl₂/Et₃SiH mixture (3 ml) for 2.5 hours to give the expected products after evaporation of the filtrate.

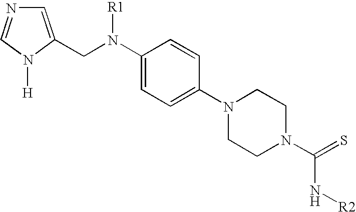

| | | | | Mass spectrum |
|---|---|---|---|---|
| Example | R1 | R2 | Compound name | (M + H)+ |
| 182 | 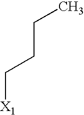 | 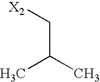 | 4-{4-[Butyl-(3H-imidazol-4- ylmethyl)amino]phenyl}-1- piperazinecarbothioic acid isobutylamide | 429 |
| 183 | 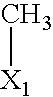 | 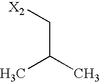 | 4-{4-[(3H-Imidazol-4- ylmethyl)methyl- amino]phenyl}-1- piperazinecarbothioic acid isobutylamide | 387 |
| 184 | 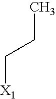 | 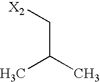 | 4-{4-[(3H-Imidazol-4- ylmethyl)propyl- amino]phenyl}-1- piperazinecarbothioic acid isobutylamide | 415 |
| 185 | 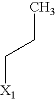 | 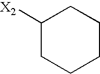 | 4-{4-[(3H-Imidazol-4- ylmethyl)propyl- amino]phenyl}-1- piperazinecarbothioic acid cyclohexylamide | 441 |
| 186 | 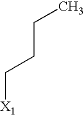 | 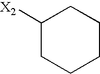 | 4-{4-[Butyl-(3H-imidazol-4- ylmethyl)amino]phenyl}-1- piperazinecarbothioic acid cyclohexylamide | 455 |

### EXAMPLES 187 TO 202

Examples 187 to 202 are prepared from compound 107 (50 mg; 0.097 mmol) according to the procedure described for the preparation of Examples 108 to 124.

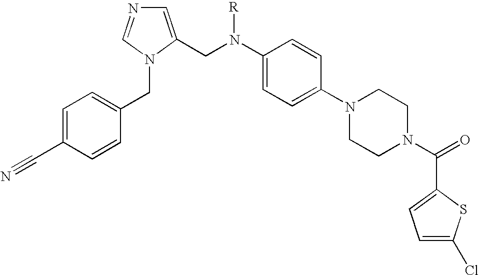

| | | | Mass |
|---|---|---|---|
| | | | spectrum |
| Example | R1 | Compound name | (M + H)+ |
| 187 | 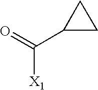 | Cyclopropanecarboxylic acid {4-[4-(5- chloro-2-thiophenecarbonyl)-1- piperazinyl]phenyl}-[3-(4-cyanobenzyl)- 3H-imidazol-4-ylmethyl]amide | 585 |
| 188 | 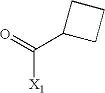 | Cyclobutanecarboxylic acid {4-[4-(5- chloro-2-thiophenecarbonyl)-1- piperazinyl]phenyl}[3-(4-cyanobenzyl)- 3H-imidazol-4-ylmethyl]amide | 599 |
| 189 | 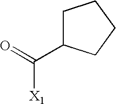 | Cyclopentanecarboxylic acid {4-[4-(5- chloro-2-thiophenecarbonyl)-1- piperazinyl]phenyl}[3-(4-cyanobenzyl)- 3H-imidazol-4-ylmethyl]amide | 613 |
| 190 | 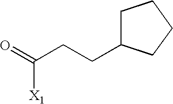 | N-{4-[4-(5-Chloro-thiophenecarbonyl)- 1-piperazinyl]phenyl}-N-[3-(4- cyanobenzyl)-3H-imidazol-4-ylmethyl]-3- cyclopentylpropionamide | 641 |
| 191 | 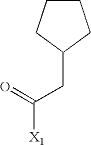 | N-{4-[4-(5-Chloro-2-thiophenecarbonyl)- 1-piperazinyl]phenyl}-N-[3-(4- cyanobenzyl)-3H-imidazol-4-ylmethyl]-2- cuyclopentylacetamide | 627 |
| 192 | 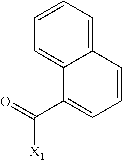 | Naphthalene-1-carboxylic acid {4-[4-(5- chloro-2-thiophenecarbonyl)-1- piperazinyl]phenyl}-[3-(4-cyanobenzyl)- 3H-imidazol-4-ylmethyl]amide | 671 |
| 193 | 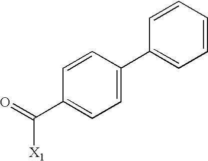 | Biphenyl-4-carboxylic acid {4-[4-(5- chloro-2-thiophenecarbonyl)-1- piperazinyl]phenyl}-[3-(4-cyanobenzyl)- 3H-imidazol-4-ylmethyl]amide | 697 |
| 194 | 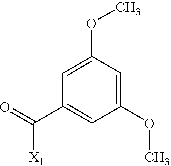 | N-{4-[4-(5-Chloro-2-thiophenecarbonyl)- 1-piperazinyl]phenylk}-N-0[3-(4- cyanobenzyl)-3H-imidazol-4-ylmethyl]- 3,5-dimethoxybenzamide | 681 |
| 195 | 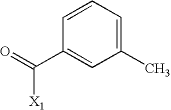 | N-{4-[4-(5-Chloro-2-thiophenecarbonyl)- 1-piperazinyl]phenyl}-N-[3-(4- cyanobenzyl)-3H-imidazol-4-ylmethyl]-3- methylbenzamide | 635 |
| 196 | 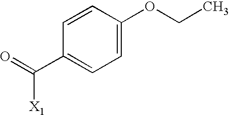 | N-{4-[4-(5-Chloro-2-thiophenecarbonyl)- 1-piperazinyl]phenyl}-N-[3-(4- cyanobenzyl)-3H-imidazol-4-ylmethyl]-4- ethoxybenzamide | 665 |
| 197 | 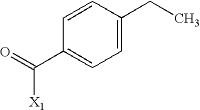 | N-{4-[4-(5-Chloro-2-thiophenecarbonyl)- 1-piperazinyl]phenyl}-N-[3-(4- cyanobenzyl)-3H-imidazol-4-ylmethyl]-4- ethylbenzamide | 649 |
| 198 | 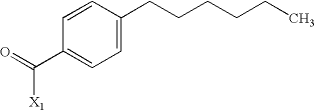 | N-{4-[4-(5-Chloro-2-thiophenecarbonyl)- 1-piperazinyl]phenyl}-N-[3-(4- cyanobenzyl)-3H-imidazol-4-ylmethyl]-4- hexylbenzamide | 705 |
| 199 | 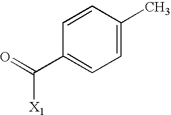 | N-{4-[4-(5-Chloro-2-thiophenecarbonyl)- 1-piperazinyl]phenyl}-N-[3-(4- cyanobenzyl)-3H-imidazol-4-ylmethyl]-4- methylbenzamide | 635 |
| 200 | 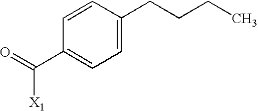 | 4-Butyl-N-{4-[4-(5-chloro-2- thiophenecarbonyl)-1-piperazinyl]phenyl- N-[3-(4-cyanobenzyl)-3H-imidazol-4- ylmethyl]benzamide | 677 |
| 201 | 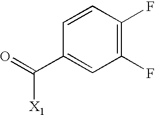 | N-{4-[4-(5-Chloro-2-thiophenecarbonyl)- 1-piperazinyl]phenyl}-N-[3-(4- cyanobenzyl)-3H-imidazol-4-ylmethyl]- 3,4-difluorobenzamide | 657 |
| 202 | 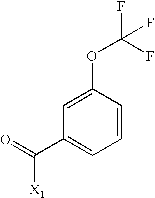 | N-{4-[4-(5-Chloro-2-thiophenecarbonyl)- 1-piperazinyl]phenyl}-N-[3-(4- cyanobenzyl)-3H-imidazol-4-ylmethyl]-3- trifluoromethoxybenzamide | 705 |

### EXAMPLES 203 TO 214

Examples 203 to 214 are prepared from compound 107 (50 mg; 0.097 mmol) according ocedure described for the preparation of Examples 108 to 124.

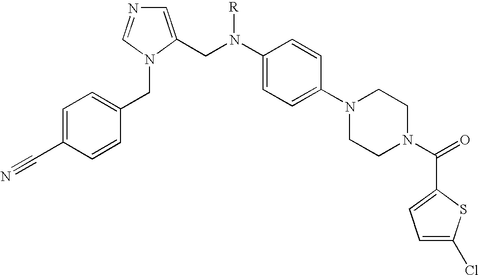

| | | | Mass |
|---|---|---|---|
| | | | spectrum |
| Example | R1 | Compound name | (M + H)+ |
| 203 | 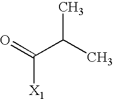 | N-{4-[4-(5-Chloro-2-thiophenecarbonyl)-1- piperazinyl]phenyl}-N-[3-(4-cyanobenzyl)- 3H-imidazol-4-ylmethyl]isobutyramide | 587 |
| 204 | 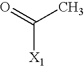 | N-{4-[4-(5-Chloro-2-thiophenecarbonyl)-1- piperazinyl]phenyl}-N-[3-(4-cyanobenzyl)- 3H-imidazol-4-ylmethyl]acetamide | 559 |
| 205 | 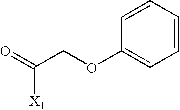 | N-{4-[4-(5-Chloro-2-thiophenecarbonyl)-1- piperazinyl]phenyl}-N-[3-(4-cyanobenzyl)- 3H-imidazol-4-ylmethyl]- 2-phenoxyacetamide | 651 |
| 206 | 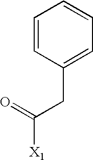 | N-{4-[4-(5-Chloro-2-thiophenecarbonyl)-1- piperazinyl]phenyl}-N-[3-(4-cyanobenzyl)- 3H-imidazol-4-ylmethyl]-2-phenylacetamide | 635 |
| 207 | 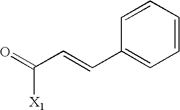 | N-{4-[4-(5-Chloro-2-thiophenecarbonyl)-1- piperazinyl]phenyl}-N-[3-(4-cyanobenzyl)- 3H-imidazol-4-ylmethyl]- 3-phenylacetamide | 647 |
| 208 | 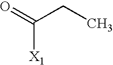 | N-{4-[4-(5-Chloro-2-thiophenecarbonyl)-1- piperazinyl]phenyl}-N-[3-(4-cyanobenzyl)- 3H-imidazol-4-ylmethyl]propionamide | 573 |
| 209 | 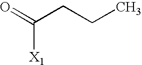 | N-{4-[4-(5-Chloro-2-thiophenecarbonyl)-1- piperazinyl]phenyl}-N-[3-(4-cyanobenzyl)- 3H-imidazol-4-ylmethyl]butyramide | 587 |
| 210 | 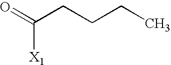 | Pentanoic acid {4-[4-(5-chloro-2- thiophenecarbonyl)-1-piperazinyl]phenyl}- [3-(4-cyanobenzyl)-3H-imidazol-4- ylmethyl]amide | 601 |
| 211 | 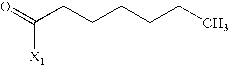 | Heptanoic acid {4-[4-(5-chloro-2- thiophenecarbonyl)-1-piperazinyl]phenyl}- [3-(4-cyanobenzyl)-3H-imidazol-4- ylmethyl]amide | 629 |
| 212 | 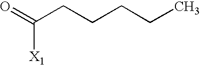 | Hexanoic acid {4-[4-(5-chloro-2- thiophenecarbonyl)-1-piperazinyl]phenyl}-[3-(4-cyanobenzyl)-3H-imidazol-4- ylmethyl]amide | 615 |
| 213 | 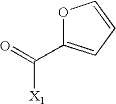 | Furan-2-carboxylic acid {4-[4-(5-chloro-2- thiophenecarbonyl)-1-piperazinyl]phenyl}- [3-(4-cyanobenzyl)-3H-imidazol-4- ylmethyl]amide | 611 |
| 214 | 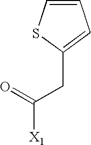 | N-{4-[4-(5-Chloro-2-thiophenecarbonyl)-1- piperazinyl]phenyl}-N-[3-(4-cyanobenzyl)- 3H-imidazol-4-ylmethyl]-2-thiophen- 2-ylacetamide | 641 |

### EXAMPLES 215 TO 218

Examples 215 to 218 are prepared from compound 107 (50 mg; 0.097 mmol) according to the procedure described for the preparation of Examples 125 to 128.

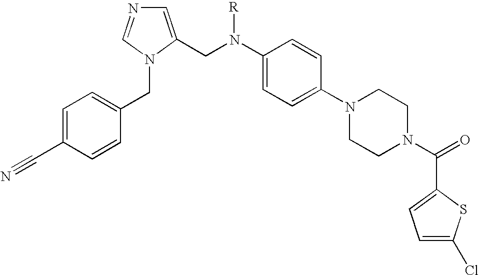

| | | | Mass |
|---|---|---|---|
| | | | spectrum |
| Example | R1 | Compound name | (M + H)+ |
| 215 | 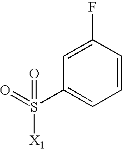 | N-{4-[4-(5-Chloro-2-thiophenecarbonyl)-1- piperazinyl]phenyl}-N-[3-(4-cyanobenzyl)- 3H-imidazol-4-ylmethyl]- 3-fluorobenzenesulfonamide | 675 |
| 216 | 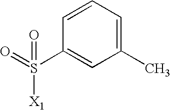 | N-{4-[4-(5-Chloro-2-thiophenecarbonyl)-1- piperazinyl]phenyl}-N-[3-(4-cyanobenzyl)- 3H-imidazol-4-ylmethyl]- 3-methylbenzenesulfonamide | 671 |
| 217 | 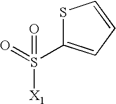 | Thiophene-2-sulfonic acid {4-[4-(5-chloro- 2-thiophenecarbonyl)-1- piperazinyl]phenyl}-[3-(4-cyanobenzyl)-3H- imidazol-4-ylmethyl]amide | 663 |
| 218 | 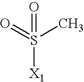 | N-{4-[4-(5-Chloro-2-thiophenecarbonyl)-1- piperazinyl]phenyl}-N-[3-(4-cyanobenzyl)- 3H-imidazol-4-ylmethyl]- methanesulfonamide | 595 |

### EXAMPLES 219 TO 226

Examples 219 to ₂₂₆ are prepared according to the following general formula: The carboxylic acids (400 mg) used for the preparation of this library are all first converted into acid chlorides by treatment with thionyl chloride (4 ml) at reflux for 5 hours. The intermediates formed are evaporated to dryness, coevaporated with dichloromethane and then dissolved in dichloromethane to a precise concentration. The acid chloride solutions are then used to treat compound 107 (50 mg; 0.097 mmol) according to the procedure described for the preparation of Examples 108 to 124.

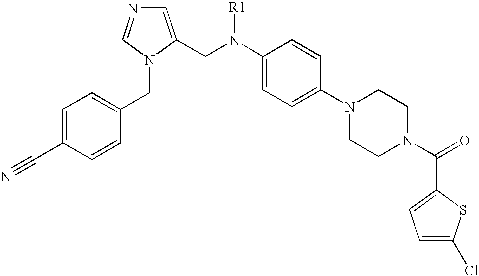

| | | | Mass |
|---|---|---|---|
| | | | spectrum |
| Example | R1 | Compound name | (M + H)+ |
| 219 | 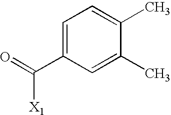 | N-{4-[4-(5-Chloro-2-thiophenecarbonyl)-1-pipera- zinyl]phenyl}-N-[3-(4-cyanobenzyl)-3H-imi- dazol-4-ylmethyl]-3,4-dimethylbenzamide | 649 |
| 220 | 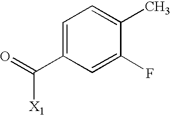 | N-{4-[4-(5-Chloro-2-thiophenecarbonyl)-1-pipera- zinyl]phenyl}-N-[3-(4-cyanobenzyl)-3H-imi- dazol-4-ylmethyl]-3-fluoro-4-methyl- benzamide | 653 |
| 221 | 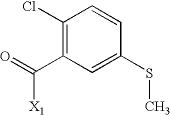 | 2-Chloro-N-{4-[4-(5-chloro-2-thiophene- carbonyl)-1-piperazinyl]phenyl}-N-[3-(4-cyano- benzyl)-3H-imidazol-4-ylmethyl]-5-methyl- sulfanylbenzamide | 701 |
| 222 | 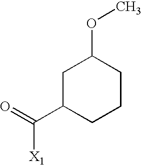 | 3-Methoxy-cyclohexanecarboxylic acid{4-[4-(5-chloro-2-thio- phenecarbonyl)-1-piperazinyl]phenyl}-[3-(4-cyano- benzyl)-3H-imidazol-4-ylmethyl]amide | 657 |
| 223 | 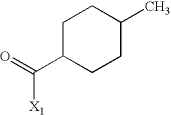 | 4-Methyl-cyclohexanecarboxylic acid{4-[4-(5-chloro-2-thio- phenecarbonyl)-1-pipera- zinyl]phenyl}[3-(4-cyanobenzyl)-3H-imi- dazol-4-ylmethyl]amide | 641 |
| 224 | 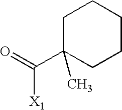 | 1-Methyl-cyclohexanecarboxylic acid{4-[4-(5-chloro-2-thio- phenecarbonyl)-1-pipera- zinyl]phenyl}[3-(4-cyano- benzyl)-3H-imidazol-4-ylmethyl]amide | 641 |
| 225 | 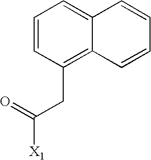 | N-{4-[4-(5-Chloro-2-thiophenecarbonyl)-1-pipera- zinyl]phenyl}-N-[3-(4-cyanobenzyl)-3H-imi- dazol-4-ylmethyl]-2-naphthalen-1-yl-aceta- mide | 685 |
| 226 | 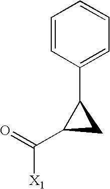 | 2-Phenyl-cyclopropanecarboxylic acid{4-[4-(5-chloro-2-thio- phenecarbonyl)-1-pipera- zinyl]phenyl}[3-(4-cyanobenzyl)-3H-imi- dazol-4-ylmethyl]amide | 661 |

### EXAMPLES ₂₂₇ TO 237

Examples 227 to 237 are prepared according to the following general procedure: Compound 107 (50 mg; 0.097 mmol) dissolved in MeOH (2.5 ml) is treated with various aldehydes (0.312 mmol) in the presence of AcOH (22 μl, 0.4 mmol) for 3 hours at room temperature. MP-cyanoborohydride resin (86 mg; 2.42 mmol/g; 0.2 mmol) is then added and the medium is stirred for 72 hours at room temperature. The medium is evaporated to dryness, taken up in dichloromethane (3 ml) and treated with PS-Trisamine resin (283 mg; 3.10 mmol). After stirring overnight at room temperature, the medium is filtered and the filtrate is evaporated to dryness to give compounds 227 to 237.

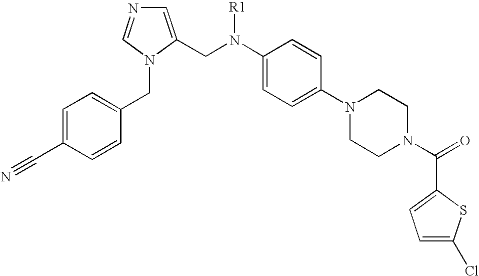

| | | | Mass |
|---|---|---|---|
| | | | spectrum |
| Example | R1 | Compound name | (M + H)+ |
| 227 | 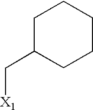 | 4-{5-[({4-[4-(5-Chloro-2-thiophenecarbonyl)-1-pipera- zinyl]phenyl}cyclohexylmethylamino)-meth- yl]imidazol-1-ylmethyl}benzonitrile | 613 |
| 228 | 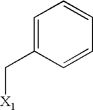 | 4-{5-[(Benzyl-{4-[4-(5-chloro-2-thio- phenecarbonyl)-1-piperazinyl]phenyl}-a- mino)methyl]imidazol-1-ylmethyl}benzonitrile | 607 |
| 229 | 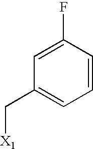 | 4-(5-{[{4-[4-(5-Chloro-2-thiophenecarbonyl)-1-pipera- zinyl]phenyl}(3-fluorobenzyl)amino]-meth- yl}imidazol-1-ylmethyl)benzonitrile | 625 |
| 230 | 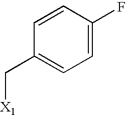 | 4-(5-{[{4-[4-(5-Chloro-2-thiophenecarbonyl)-1-pipera- zinyl]phenyl}-(4-fluorobenzyl)amino]-meth- yl}imidazol-1-ylmethyl)benzonitrile | 625 |
| 231 | 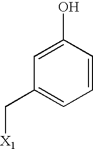 | 4-(5-{[{4-[4-(5-Chloro-2-thiophenecarbonyl)-1-pipera- zinyl]phenyl}(3-hydroxybenzyl)amino]-meth- yl}imidazol-1-ylmethyl)benzonitrile | 623 |
| 232 | 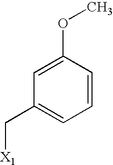 | 4-(5-{[{4-[4-(5-Chloro-2-thiophenecarbonyl)-1-pipera- zinyl]phenyl}(3-methoxy-benzyl)amino]-meth- yl}imidazol-1-ylmethyl)benzonitrile | 637 |
| 233 | 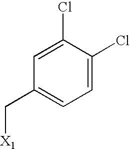 | 4-(5-{[{4-[4-(5-Chloro-2-thiophenecarbonyl)-1-pipera- zinyl]phenyl}-(3,4-dichlorobenzyl)-a- mino]methyl}imidazol-1-ylmethyl)benzonitrile | 675-677 |
| 234 | 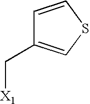 | 4-{5-[({4-[4-(5-Chloro-2-thiophenecarbonyl)-1-pipera- zinyl]phenyl}thiophen-3-ylmethylamino)-meth- yl]imidazol-1-ylmethyl}benzonitrile | 613 |
| 235 | 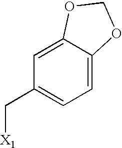 | 4-{5-[(Benzo[1,3]dioxol-5-ylmethyl-{4-[4-(5-chloro-2-thio- phenecarbonyl)-1-pipera- zinyl]phenyl}amino)methyl]imidazol-1-ylmeth- yl}benzonitrile | 651 |
| 236 | 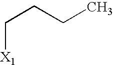 | 4-{5-[(Butyl-{4-[4-(5-chloro-2-thio- phenecarbonyl)-1-piperazinyl]phenyl}-a- mino)methyl]imidazol-1-ylmethyl}benzonitrile | 573 |
| 237 | 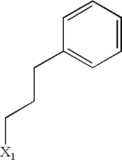 | 4-(5-{[{4-[4-(5-Chloro-2-thiophenecarbonyl)-1-pipera- zinyl]phenyl}-(3-phenylpropyl)amino]-meth- yl}imidazol-1-ylmethyl)benzonitrile | 635 |

### EXAMPLES 238 TO 246

Examples 238 to 246 are prepared from compound 106 (50 mg; 0.104 mmol) according to the procedure described for the preparation of Examples 227 to 237.

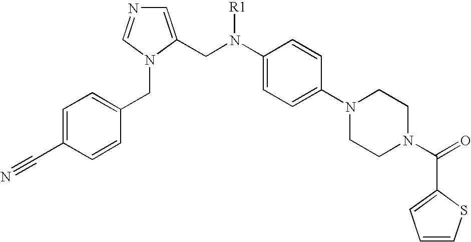

| | | | Mass |
|---|---|---|---|
| | | | spectrum |
| Example | R1 | Compound name | (M + H)+ |
| 238 | 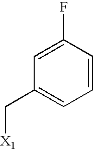 | 4-{5-[((3-Fluorobenzyl){4-[4-(2-thiophene-carbo- nyl)-1-piperazinyl]phenyl}amino)-meth- yl]imidazol-1-ylmethyl}benzonitrile | 591 |
| 239 | 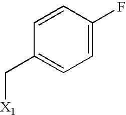 | 4-{5-[((4-Fluorobenzyl)-{4-[4-(2-thiophene-carbo- nyl)-1-piperazinyl]phenyl}amino)-meth- yl]imidazol-1-ylmethyl}benzonitrile | 591 |
| 240 | 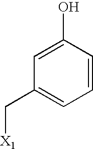 | 4-{5-[((4-Fluorobenzyl)-{4-[4-(2-thiophene-carbo- nyl)-1-piperazinyl]phenyl}amino)-meth- yl]imidazol-1-ylmethyl}benzonitrile | 589 |
| 241 | 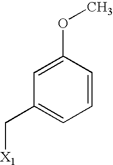 | 4-{5-[((3-Methoxybenzyl)-{4-[4-(thiophene-2-carbo- nyl)-1-piperazinyl]phenyl}amino)-meth- yl]imidazol-1-ylmethyl}benzonitrile | 603 |
| 242 | 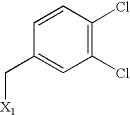 | 4-{5-[((3,4-Dichlorobenzyl)-{4-[4-(thio- phene-2-carbonyl)-1-pipera- zinyl]phenyl}amino)methyl]imidazol-1-ylmeth- yl}benzonitrile | 641 |
| 243 | 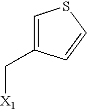 | 4-{5-[({4-[4-(2-Thiophenecarbonyl)-1-pipera- zinyl]phenyl}thiophen-3-ylmethyl- amino)methyl]imidazol-1-ylmethyl}-benzonitrile | 579 |
| 244 | 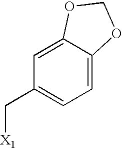 | 4-{5-[(Benzo[1,3]dioxol-5-ylmethyl-{4-[4-(thio- phene-2-carbonyl)-1-pipera- zinyl]phenyl}amino)methyl]imidazol-1-ylmeth- yl}benzonitrile | 617 |
| 245 | 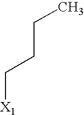 | 4-{5-[(Butyl-{4-[4-(thiophene-2-carbonyl)-1-pipera- zinyl]phenyl}amino)methyl]imidazol-1-ylmeth- yl}benzonitrile | 539 |
| 246 | 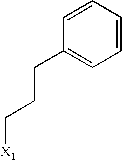 | 4-{5-[((3-Phenyl-propyl)-{4-[4-(thiophene-2-carbo- nyl)-1-piperazinyl]phenyl}amino)-meth- yl]imidazol-1-ylmethyl}benzonitrile | 601 |

### EXAMPLE 247

### 4-{5-[(4-1-piperazinyl-phenylamino)methyl]imidazol-1-ylmethyl}benzonitrile

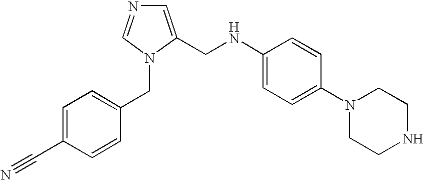

### EXAMPLE 247A

### 4-(4-{[3-(4-Cyanobenzyl)-3H-imidazol-4-ylmethyl]amino}phenyl)-piperazine-1carboxylic acid 9H-fluoren-9-ylmethyl ester

A mixture of compound 106A (3.7 g; 17.52 mmol) and compound 2B (7 g; 17.52 mmol) dissolved in dichloroethane (90 ml) in the presence of AcOH (5.5 ml; 105.1 mmol) is stirred for 5 minutes at room temperature and then treated with NaBH(OAc)₃ (4.1 g; 19.27 mmol). The mixture is stirred overnight at room temperature and is then diluted in EtOAc and washed with saturated NaHCO₃ solution, with water and with saturated NaCl solution. The organic phase is dried over Na₂SO₄ and evaporated to dryness. The product obtained in the form of a brown syrup is purified on a column of silica eluted with a 97.75/2/0.25 CH₂Cl₂/MeOH/NH₄OH mixture to give the pure product in the form of a beige-colored foam (5.68 g; 55%).

Mass spectrum (ESI): m/z 595 (MH⁺).

### EXAMPLE 247

### 4-{5-[(4-1-piperazinyl-phenylamino)methyl]imidazol-1-ylmethyl}-benzonitrile

Compound 247A (50 mg; 0.084 mmol) dissolved in DMF (1 ml) is treated with a 5% solution of piperidine in DMF (50 μl). The mixture is stirred for 5 minutes at room temperature and is then diluted with EtOAc and washed with water and with saturated NaCl solution. The organic phase is dried over Na₂SO₄ and evaporated to dryness. The product obtained in the form of a yellow syrup is purified on a column of silica eluted with a 90/9/1 CH₂Cl₂/MeOH/NH₄OH mixture to give a pure product in the form of yellow crystals (14 mg; 45%).

Mass spectrum (ESI): m/z 373 (MH⁺).

### EXAMPLE 248

### N-[3-(4-Cyanobenzyl)-3H-imidazol-4-ylmethyl]-N-(4-1-piperazinylphenyl)-benzenesulfonamide

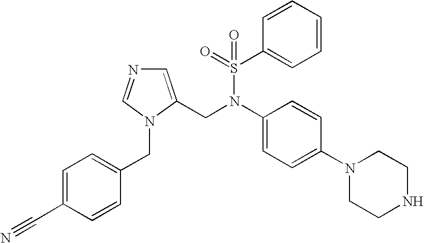

Compound 248 is prepared from compound 247A (100 mg; 0.168 mmol) according to the procedure described for the preparation of Examples 125 to 128. The intermediate obtained is then deprotected according to the procedure described for the preparation of Example 247, starting with 247A.

Mass spectrum (ESI): m/z 513 (MH⁺).

### EXAMPLE 249

### 4-Cyano-N-[3-(4-cyanobenzyl)-3H-imidazol-4-ylmethyl]-N-(4-1-piperazinylphenyl)-benzenesulfonamide

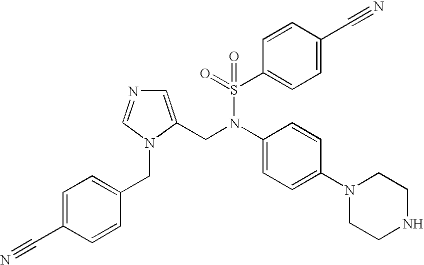

Compound 249 is prepared from compound 247A (585 mg; 0.984 mmol) and 4-cyanobenzenesulfonyl chloride (298 mg; 1.476 mmol) according to the procedure described for the preparation of Example 248.

Mass spectrum (ESI): m/z 760 (MH⁺).

### EXAMPLE 250

### 4-Cyano-N-[3-(4-cyanobenzyl)-3H-imidazol-4-ylmethyl]-N-(4-1-piperazinylphenyl)-benzamide

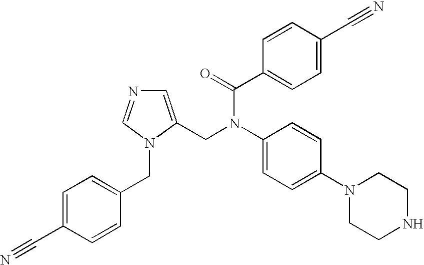

Compound 250 is prepared from compound 247A (585 mg; 0.984 mmol) and 4-cyanobenzoyl chloride (244 mg; 1.476 mmol) according to the procedure described for the preparation of Example 248.

Mass spectrum (ESI): m/z 724 (MH⁺).

### EXAMPLE 251

### N-[3-(4-Cyanobenzyl)-3H-imidazol-4-ylmethyl]-3-fluoro-N-(4-1-piperazinylphenyl)-benzamide

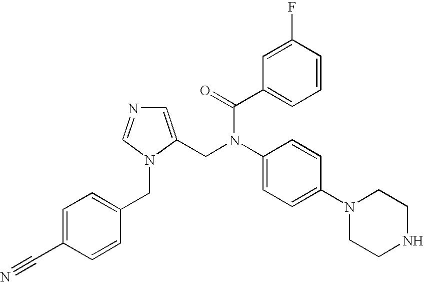

Compound 251 is prepared from compound 247A (585 mg; 0.984 mmol) and 3-fluorobenzoyl chloride (180 μl; 1.476 mmol) according to the procedure described for the preparation of Example 248.

Mass spectrum (ESI): m/z 717 (MH⁺).

### EXAMPLES 252 TO 271

Compounds 252 to 271 are prepared according to the following general procedure: Compounds 248 to 251 (0.035 to 0.082 mmol) dissolved in toluene (2 ml) are treated with various thioisocyanates (1.5 eq) and then heated at 60° C. for 3.3 hours. PS-trisamine resin (5 eq; 4.71 mmol/g) is then added and the mixtures are stirred overnight at room temperature. Each medium is filtered and the filtrate is evaporated to dryness to give compounds 252 to 271.

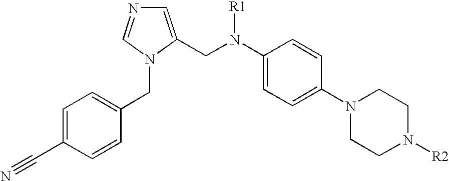

| | | | | Mass |
|---|---|---|---|---|
| | | | | spectrum |
| Example | R1 | R2 | Compound name | (M + H)+ |
| 252 | 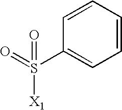 | 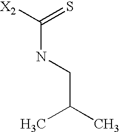 | 4-(4-{Benzenesulfonyl-[3-(4-cyano- benzyl)-3H-imidazol-4-ylmeth- yl]amino}phenyl)-1-pipera- zinecarbothioic acid isobutylamide | 628 |
| 253 | 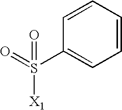 | 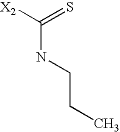 | 4-(4-{Benzenesulfonyl-[3-(4-cyano- benzyl)-3H-imidazol-4-ylmeth- yl]amino}phenyl)-1-pipera- zinecarbothioic acid propylamide | 614 |
| 254 | 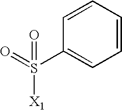 | 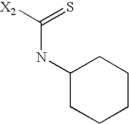 | 4-(4-{Benzenesulfonyl-[3-(4-cyano- benzyl)-3H-imidazol-4-ylmeth- yl]amino}phenyl)-1-pipera- zinecarbothioic acid cyclohexylamide | 654 |
| 255 | 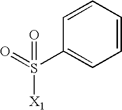 | 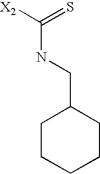 | 4-(4-{Benzenesulfonyl-[3-(4-cyano- benzyl)-3H-imidazol-4-ylmeth- yl]amino}phenyl)-1-pipera- zinecarbothioic acid cyclohexylmethylamide | 668 |
| 256 | 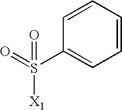 | 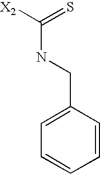 | 4-(4-{Benzenesulfonyl-[3-(4-cyano- benzyl)-3H-imidazol-4-ylmeth- yl]amino}phenyl)-1-pipera- zinecarbothioic acid benzylamide | 662 |
| 257 | 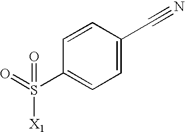 | 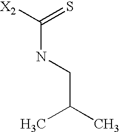 | 4-(4-{(4-Cyanobenzene- sulfonyl)-[3-(4-cyano- benzyl)-3H-imidazol-4-ylmeth- yl]amino}phenyl)-1-pipera- zinecarbothioic acid isobutylamide | 653 |
| 258 | 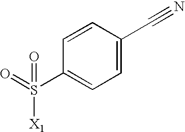 | 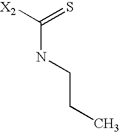 | 4-(4-{(4-Cyano- benzenesulfonyl)-[3-(4-cyano- benzyl)-3H-imidazol-4-ylmeth- yl]amino}phenyl)-1-pipera- zinecarbothioic acid propylamide | 639 |
| 259 | 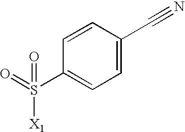 | 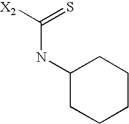 | 4-(4-{(4-Cyano- benzenesulfonyl)-[3-(4-cyano- benzyl)-3H-imidazol-4-ylmeth- yl]amino}phenyl)-1-pipera- zinecarbothioic acid cyclohexylamide | 679 |
| 260 | 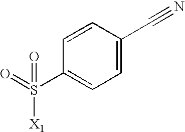 | 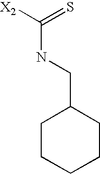 | 4-(4-{(4-Cyano- benzenesulfonyl)-[3-(4-cyano- benzyl)-3H-imidazol-4-ylmeth- yl]amino}phenyl)-1-pipera- zinecarbothioic acid cyclohexylmethylamide | 693 |
| 261 | 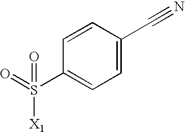 | 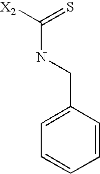 | 4-(4-{(4-Cyano- benzenesulfonyl)-[3-(4-cyano- benzyl)-3H-imidazol-4-ylmeth- yl]amino}phenyl)-1-pipera- zinecarbothioic acid benzylamide | 687 |
| 262 | 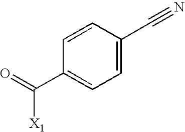 | 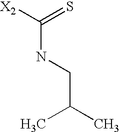 | 4-Cyano-N-[3-(4-cyano- benzyl)-3H-imidazol-4-ylmeth- yl]-N-[4-(4-iso- butylthiocarbamoyl-1-pipera- zinyl)phenyl]-benzamide | 617 |
| 263 | 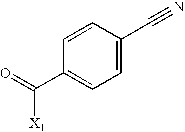 | 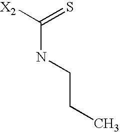 | 4-Cyano-N-[3-(4-cyano- benzyl)-3H-imidazol-4-ylmeth- yl]-N-[4-(4-propyl- thiocarbamoyl-1-pipera- zinyl)phenyl]-benzamide | 603 |
| 264 | 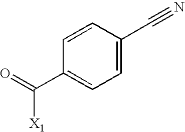 | 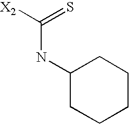 | 4-Cyano-N-[3-(4-cyano- benzyl)-3H-imidazol-4-ylmeth- yl]-N-[4-(4-cyclo- hexylthiocarbamoyl-1-pipera- zinyl)phenyl]-benzamide | 643 |
| 265 | 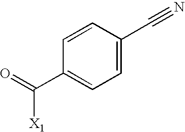 | 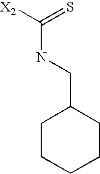 | 4-Cyano-N-[3-(4-cyano- benzyl)-3H-imidazol-4-ylmeth- yl]-N-{4-[4-(cyclo- hexylmethyl- thiocarbamoyl)-1-pipera- zinyl]phenyl}-benzamide | 657 |
| 266 | 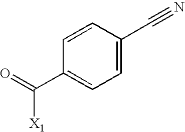 | 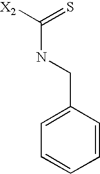 | N-[4-(4-Benzylthio- carbamoyl-1-pipera- zinyl)-phenyl]-4-cyano-N-[3-(4-cyano- benzyl)-3H-imidazol-4-ylmethyl]benzamide | 651 |
| 267 | 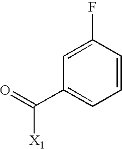 | 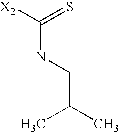 | N-[3-(4-Cyanobenzyl)-3H-imi- dazol-4-ylmethyl]-3-fluoro-N-[4-(4-iso- butylthiocarbamoyl-1-pipera- zinyl)phenyl]-benzamide | 610 |
| 268 | 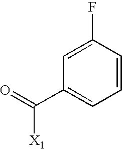 | 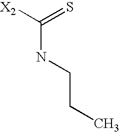 | N-[3-(4-Cyano-benzyl)-3H-imi- dazol-4-ylmethyl]-3-fluoro-N-[4-(4-propyl- thiocarbamoyl-1-pipera- zinyl)phenyl]-benzamide | 596 |
| 269 | 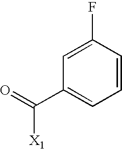 | 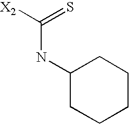 | N-[3-(4-Cyanobenzyl)-3H-imi- dazol-4-ylmethyl]-N-[4-(4-cyclo- hexylthiocarbamoyl-1-pipera- zinyl)phenyl]-3-fluorobenzamide | 636 |
| 270 | 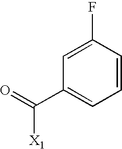 | 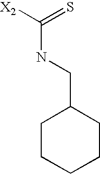 | N-[3-(4-Cyanobenzyl)-3H-imi- dazol-4-ylmethyl]-N-{4-[4-(cyclo- hexylmethyl- thiocarbamoyl)-1-pipera- zinyl]phenyl}-3-fluorobenzamide | 650 |
| 271 | 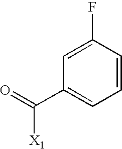 | 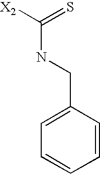 | N-[4-(4-Benzyl- thiocarbamoyl-1-pipera- zinyl)phenyl]-N-[3-(4-cyano- benzyl)-3H-imidazol-4-ylmeth- yl]-3-fluorobenzamide | 644 |

### EXAMPLE 272

### 1-[3-(4-Cyanobenzyl)-3H-imidazol-4-ylmethyl]-3-(2-methylsulfanylphenyl)-1-(4-1-piperazinylphenyl)urea

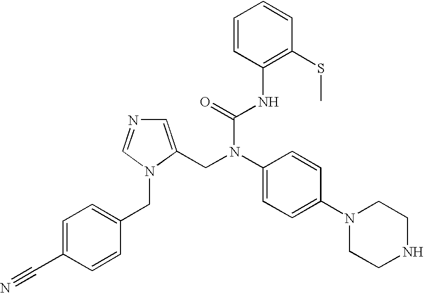

Intermediate 247A (585 mg; 0.984 mmol) in toluene (12 ml) is treated with 2-(methyltio)phenyl isocyanate (244 mg; 1.476 mmol) at 60° C. for 1 hour 30 minutes. The medium is cooled to room temperature and then treated with PS-trisamine resin (1 g; 4.71 mmol/g; 4.71 mmol) and stirred at room temperature for 4 hours. The resin is filtered off and the filtrate is evaporated to dryness to give the intermediate product, which is deprotected under the conditions described for the preparation of Exemple 247, starting with 247A, to give the desired product.

Mass spectrum (ESI): m/z 538 (MH⁺).

### EXAMPLE 273

### 1-[3-(4-Cyanobenzyl)-3H-imidazol-4-ylmethyl]-1-(4-1-piperazinyl-phenyl)-3-propylurea

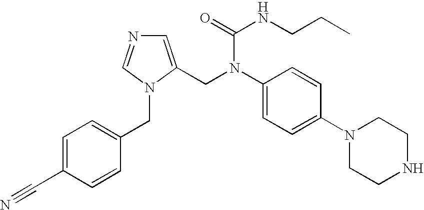

Compound 273 is prepared from compound 247A (585 mg; 0.984 mmol) and propyl isocyanate (139 μl; 1.476 mmol) according to the procedure described for the preparation of Example 272.

Mass spectrum (ESI): mn/z 458 (MH⁺).

### EXAMPLES 274 TO 283

Examples 274 to 283 are prepared from compound 272 (40 mg; 0.074 mmol) or 273 (40 mg; 0.087 mmol) according to the procedure described for the preparation of Examples 252 to 271.

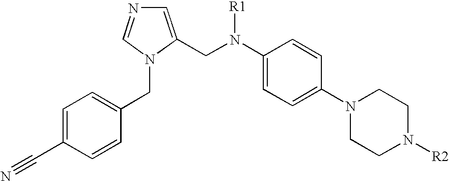

| | | | | Mass |
|---|---|---|---|---|
| | | | | spectrum |
| Example | R1 | R2 | Compound name | (M + H)+ |
| 274 | 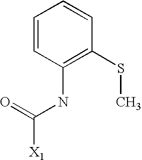 | 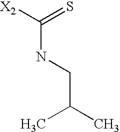 | 4-{4-[1-[3-(4-Cyanobenzyl)-3H-imi- dazol-4-ylmethyl]-3-(2-methyl- sulfanylphenyl)-ureido]phenyl}-1-pipera- zinecarbothioic acid isobutylamide | 653 |
| 275 | 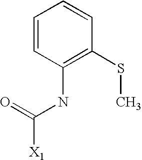 | 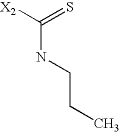 | 4-{4-[1-[3-(4-Cyanobenzyl)-3H-imi- dazol-4-ylmethyl]-3-(2-methyl- sulfanylphenyl)-ureido]phenyl}-1-pipera- zinecarbothioic acid propylamide | 639 |
| 276 | 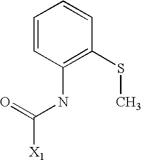 | 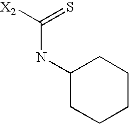 | 4-{4-[1-[3-(4-Cyanobenzyl)-3H-imi- dazol-4-ylmethyl]-3-(2-methyl- sulfanylphenyl)-ureido]phenyl}-1-piperazine- carbothioic acid cyclohexylamide | 679 |
| 277 | 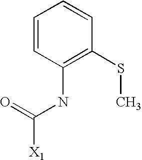 | 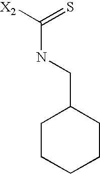 | 4-{4-[1-[3-(4-Cyanobenzyl)-3H-imi- dazol-4-ylmethyl]-3-(2-methyl- sulfanylphenyl)-ureido]phenyl}-1-piperazine- carbothioic acid cyclohexylmethylamide | 693 |
| 278 | 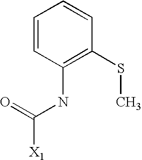 | 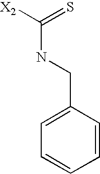 | 4-{4-[1-[3-(4-Cyanobenzyl)-3H-imi- dazol-4-ylmethyl]-3-(2-methyl- sulfanylphenyl)-ureido]phenyl}-1-piperazine- carbothioic acid benzylamide | 687 |
| 279 | 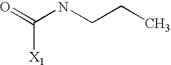 | 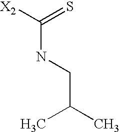 | 4-(4-{1-[3-(4-Cyanobenzyl)-3H-imi- dazol-4-ylmethyl]-3-propyl-ureido}phenyl)-1-piperazine- carbothioic acid isobutylamide | 573 |
| 280 | 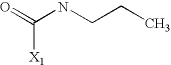 | 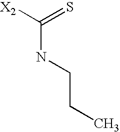 | 4-(4-{1-[3-(4-Cyanobenzyl)-3H-imi- dazol-4-ylmethyl]-3-propyl-ureido}phenyl)-1-piperazine- carbothioic acid propylamide | 559 |
| 281 | 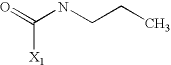 | 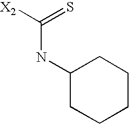 | 4-(4-{1-[3-(4-Cyanobenzyl)-3H-imi- dazol-4-ylmethyl]-3-propyl-ureido}phenyl)-1-piperazine- carbothioic acid cyclohexylamide | 599 |
| 282 | 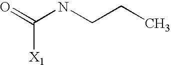 | 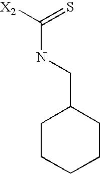 | 4-(4-{1-[3-(4-Cyanobenzyl)-3H-imi- dazol-4-ylmethyl]-3-propyl-ureido}phenyl)-1-piperazine- carbothioic acid cyclohexylmethylamide | 613 |
| 283 | 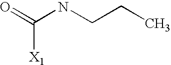 | 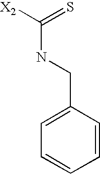 | 4-(4-{1-[3-(4-Cyanobenzyl)-3H-imi- dazol-4-ylmethyl]-3-propyl-ureido}phenyl)-1-piperazine- carbothioic acid benzylamide | 607 |

### EXAMPLE 284

### 4-(5-{[Benzyl-(4-1-piperazinylphenyl)amino]methyl}-1-imidazolylmethyl)-benzonitrile

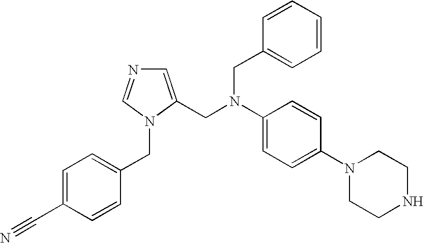

Compound 284 protected with an Fmoc group is prepared from compound 247A (585 mg; 0.984 mmol) and benzaldehyde (500 μl; 4.92 mmol) according to the procedure described for the preparation of compounds 227 to 237. The deprotection is then performed under the conditions described for the preparation of Example 247 starting with 247A, to give the desired product.

Mass spectrum (ESI): m/z 685 (MH⁺).

### EXAMPLE 285

### 4-(5-{[Cyclohexylmethyl-(4-1-piperazinylphenyl)amino]methyl}-1-imidazolylmethyl)benzonitrile

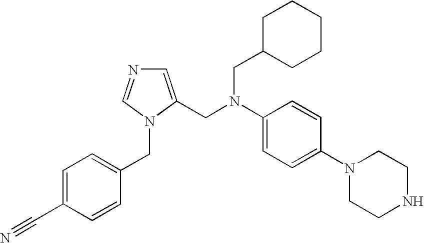

Compound 285 is prepared from compound 247A (585 mg; 0.984 mmol) and cyclohexanecarboxaldehyde (500 μl; 4.92 mmol) according to the procedure described for the preparation of Example 284.

Mass spectrum (ESI): m/z 469 (MH⁺).

### EXAMPLES 286 TO 295

Examples 286 to 295 are prepared from compound 284 (42.6 mg; 0.092 mmol) or 285 (40 mg; 0.085 mmol) according to the procedure described for the preparation of Examples 252 to 271.

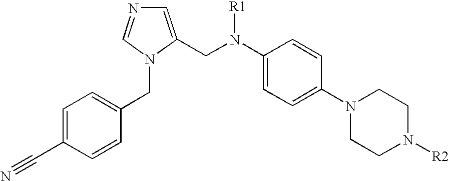

| | | | | Mass |
|---|---|---|---|---|
| | | | | spectrum |
| Example | R1 | R2 | Compound name | (M + )+ |
| 286 | 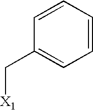 | 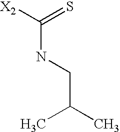 | 4-(4-{Benzyl-[3-(4-cyano- benzyl)-3H-imidazol-4-ylmeth- yl]amino}phenyl)-1-pipera- zinecarbothioic acid isobutylamide | 578 |
| 287 | 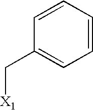 | 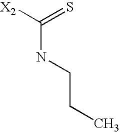 | 4-(4-{Benzyl-[3-(4-cyano- benzyl)-3H-imidazol-4-ylmeth- yl]amino}phenyl)-1-pipera- zinecarbothioic acid propylamide | 564 |
| 288 | 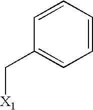 | 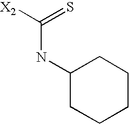 | 4-(4-{Benzyl-[3-(4-cyano- benzyl)-3H-imidazol-4-ylmeth- yl]amino}phenyl)-1-pipera- zinecarbothioic acid cyclohexylamide | 604 |
| 289 | 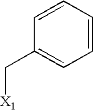 | 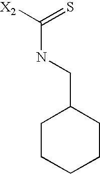 | 4-(4-{Benzyl-[3-(4-cyano- benzyl)-3H-imidazol-4-ylmeth- yl]amino}phenyl)-1-pipera- zinecarbothioic acid cyclohexylmethylamide | 618 |
| 290 | 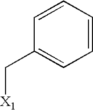 | 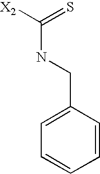 | 4-(4-{Benzyl-[3-(4-cyano- benzyl)-3H-imidazol-4-ylmeth- yl]amino}phenyl)-1-pipera- zinecarbothioic acid benzylamide | 612 |
| 291 | 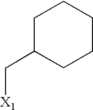 | 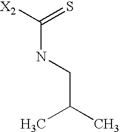 | 4-(4-{[3-(4-Cyanobenzyl)-3H-imi- dazol-4-ylmethyl]-cyclo- hexylmethylamino}-phenyl)-1-piperazine- carbothioic acid isobutylamide | 584 |
| 292 | 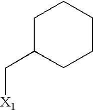 | 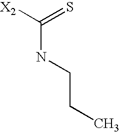 | 4-(4-{[3-(4-Cyanobenzyl)-3H-imi- dazol-4-ylmethyl]-cyclo- hexylmethylamino}-phenyl)-1-piperazine- carbothioic acid propylamide | 570 |
| 293 | 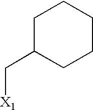 | 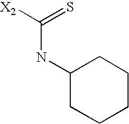 | 4-(4-{[3-(4-Cyanobenzyl)-3H-imi- dazol-4-ylmethyl]-cyclo- hexylmethylamino}-phenyl)-1-piperazine- carbothioic acid cyclohexylamide | 610 |
| 294 | 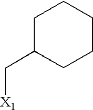 | 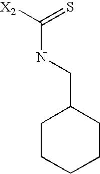 | 4-(4-{[3-(4-Cyanobenzyl)-3H-imi- dazol-4-ylmethyl]-cyclo- hexylmethylamino}-phenyl)-1-pipera- zinecarbothioic acid cyclohexylmethyl-amide | 624 |
| 295 | 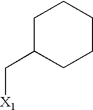 | 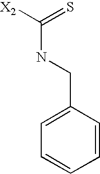 | 4-(4-{[3-(4-Cyanobenzyl)-3H-imi- dazol-4-ylmethyl]-cyclo- hexylmethylamino}-phenyl)-1-piperazine- carbothioic acid benzylamide | 618 |

### EXAMPLE 296

### N-[3(4-Cyanobenzyl)-3H-imidazol4-ylmethyl]-N-(4-1-piperazinylphenyl)benzamide

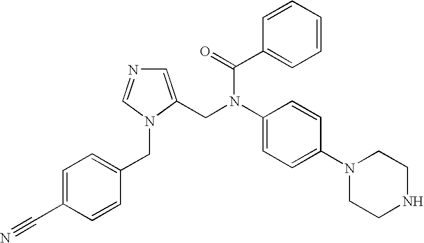

Compound 296 is prepared from compound 247A (3.0 g; 5.04 mmol) and benzoyl chloride (180 μl; 1.476 mmol) according to the procedure described for the preparation of Example 248.

Mass spectrum (ESI): m/z 477 (MH⁺).

### EXAMPLES 297 TO 303

Examples 297 to 303 are prepared from compound 296 (50 mg; 0.105 mmol) according to the procedure described for the preparation of Examples 108 to 124.

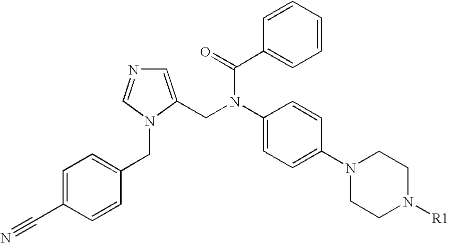

| | | | Mass |
|---|---|---|---|
| | | | spectrum |
| Example | R1 | Compound name | (M + H)+ |
| 297 | 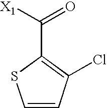 | N-{4-[4-(3-Chloro-2-thiophenecarbonyl)-1-pipera- zinyl]phenyl}-N-[3-(4-cyanobenzyl)-3H-imi- dazol-4-ylmethyl]benzamide | 621 |
| 298 | 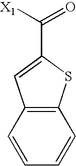 | N-{4-[4-(Benzo[b]-2-thiophenecarbonyl)-1-pipera- zinyl]phenyl}-N-[3-(4-cyanobenzyl)-3H-imi- dazol-4-ylmethyl]benzamide | 637 |
| 299 | 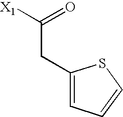 | N-{4-[4-(3-Chlorobenzo[b]-2-thio- phenecarbonyl)-1-piperazinyl]phenyl}-N-[3-(4-cyano- benzyl)-3H-imidazol-4-ylmethyl]benzamide | 671 |
| 300 | 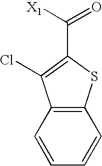 | N-[3-(4-Cyanobenzyl)-3H-imidazol-4-ylmeth- yl]-N-{4-[4-(2-thiophen-2-ylacetyl)-1-pipera- zinyl]phenyl}benzamide | 601 |
| 301 | 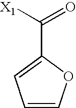 | N-[3-(4-Cyanobenzyl)-3H-imidazol-4-ylmeth- yl]-N-{4-[4-(2-furancarbonyl)-1-pipera- zinyl]phenyl}benzamide | 571 |
| 302 | 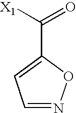 | N-[3-(4-Cyanobenzyl)-3H-imidazol-4-ylmeth- yl]-N-{4-[4-(5-isoxazolecarbonyl)-1-pipera- zinyl]phenyl}benzamide | 572 |
| 303 | 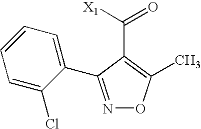 | N-(4-{4-[3-(2-Chlorophenyl)-5-methyl-4-isoxazole- carbonyl]-1-piperazinyl}phenyl)-N-[3-(4-cyano- benzyl)-3H-imidazol-4-ylmethyl]benzamide | 696 |

### EXAMPLES 304 TO 316

Examples 304 to 316 are prepared according to the following general procedure: Various carboxylic acids (0.158 mmol) dissolved in dichloromethane (3 ml) are treated with PS-carbodiimide (200 mg; 1.05 mmol/g; 0.210 mmol) and HOBT (24 mg; 0.178 mmol). After stirring for 30 minutes at room temperature, compound 296 (50 mg; 0.105 mmol) is added to each of the mixtures and the media are stirred for 4 hours at room temperature. MP-Carbonate resin (200 mg; 2.64 mmol/g; 0.52 mmol) is then added and the media are stirred overnight at room temperature. The various reactions are filtered, the resins are washed with dichloromethane and MeOH and the filtrates are evaporated to dryness. The products obtained are purified on columns of silica eluted with a 95/5 to 90/10 and then 80/20 CH₂Cl₂/MeOH gradient to give compounds 304 to 316.

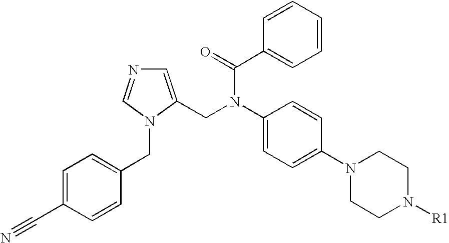

| | | | Mass |
|---|---|---|---|
| | | | spectrum |
| Example | R1 | Compound name | (M + H)+ |
| 304 | 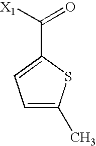 | N-[3-(4-Cyanobenzyl)-3H-imidazol-4-ylmeth- yl]-N-{4-[4-(5-methyl-2-thiophene- carbonyl)-1-piperazinyl]phenyl}-benzamide | 601 |
| 305 | 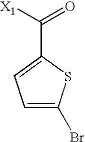 | N-{4-[4-(5-Bromo-2-thiophenecarbonyl)-1-pipera- zinyl]phenyl}-N-[3-(4-cyanobenzyl)-3H-imi- dazol-4-ylmethyl]benzamide | 665 and 667 |
| 306 | 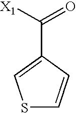 | N-[3-(4-Cyanobenzyl)-3H-imidazol-4-ylmeth- yl]-N-{4-[4-(3-thiophenecarbonyl)-1-pipera- zinyl]phenyl}benzamide | 587 |
| 307 | 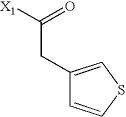 | N-[3-(4-Cyanobenzyl)-3H-imidazol-4-ylmeth- yl]-N-{4-[4-(2-thiophen-3-ylacetyl)-1-pipera- zinyl]phenyl}benzamide | 601 |
| 308 | 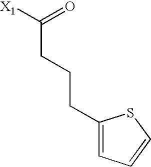 | N-[3-(4-Cyanobenzyl)-3H-imidazol-4-ylmeth- yl]-N-{4-[4-(4-thiophen-2-ylbutyryl)-1-pipera- zinyl]phenyl}benzamide | 629 |
| 309 | 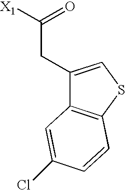 | N-(4-{4-[2-(5-Chlorobenzo[b]thiophen-3-yl)-acetyl]-1-pipera- zinyl}phenyl)-N-[3-(4-cyano- benzyl)-3H-imidazol-4-ylmethyl]benzamide | 685 |
| 310 | 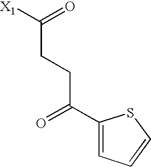 | N-[3-(4-Cyano-benzyl)-3H-imidazol-4-ylmeth- yl]-N-{4-[4-(4-oxo-4-thiophen-2-yl-butyryl)-1-pipera- zinyl]phenyl}benzamide | 643 |
| 311 | 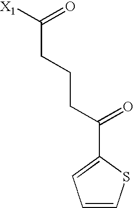 | N-[3-(4-Cyano-benzyl)-3H-imidazol-4-ylmeth- yl]-N-{4-[4-(5-oxo-5-thiophen-2-yl-penta- noyl)-1-piperazinyl]phenyl}benzamide | 657 |
| 312 | 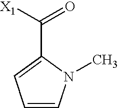 | N-[3-(4-Cyano-benzyl)-3H-imidazol-4-ylmeth- yl]-N-{4-[4-(1-methyl-1H-pyrrole-2-carbo- nyl)-1-piperazinyl]phenyl}benzamide | 584 |
| 313 | 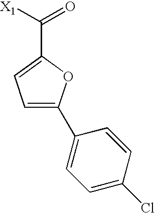 | N-(4-{4-[5-(4-Chlorophenyl)-2-furan- carbonyl]-1-piperazinyl}phenyl)-N-[3-(4-cyano- benzyl)-3H-imidazol-4-ylmethyl]benzamide | 681 |
| 314 | 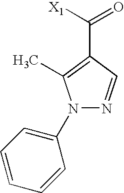 | N-[3-(4-Cyanobenzyl)-3H-imidazol-4-ylmeth- yl]-N-{4-[4-(5-methyl-1-phenyl-1H-py- razol-4-carbonyl)-1-pipera- zinyl]phenyl}benzamide | 661 |
| 315 | 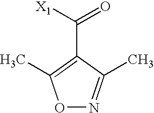 | N-[3-(4-Cyanobenzyl)-3H-imidazol-4-ylmeth- yl]-N-{4-[4-(3,5-dimethylisoxazole-4-carbo- nyl)-1-piperazinyl]phenyl}benzamide | 600 |
| 316 | 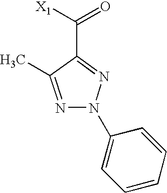 | N-[3-(4-Cyanobenzyl)-3H-imidazol-4-ylmeth- yl]-N-{4-[4-(5-methyl-2-phenyl-2H-[1,2,3]tria- zole-4-carbonyl)-1-piperazinyl]phenyl}benzamide | 662 |

### EXAMPLES 317 TO 320

### N-{4-[4-(5-Chloro-2-thiophenecarbonyl)-1-piperazinyl]phenyl}-N-(1-methyl-1H-benzoimidazol-2-ylmethyl)benzamide

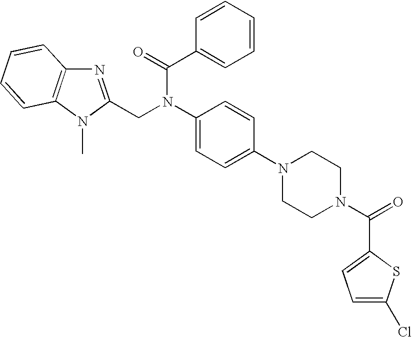

### EXAMPLE 317A

### (5-Chlorothiophen-2-yl)-(4-{4-[(1-methyl-1H-benzoimidazol-2-ylmethyl)amino]phenyl}-1-piperazinyl)methanone

Compound 317A is prepared from 1-methyl-2-formylbenzimidazole (37 mg; 0.233 mmol) and compound 57A (75 mg; 0.233 mmol) according to the conditions used in the preparation of Example 106.

### EXAMPLE 317

### N-{4-[4-(5-Chloro-2-thiophenecarbonyl)-1-piperazinyl]phenyl}-N-(1-methyl-1H-benzoimidazol-2-ylmethyl)benzamide

Intermediate 317A (37 mg; 0.079 mmol) is treated with benzoyl chloride (16 μl; 0.134 mmol) under the conditions described for the preparation of Examples 125 to 128, to give compound 317.

Mass spectrum (ESI): m/z 570 (MH⁺).

### EXAMPLE 318

### N-{4-[4-(5-Chloro-2-thiophenecarbonyl)-1-piperazinyl]phenyl}-N-(2-methyl-3-Himidazol-4-ylmethyl)benzamide

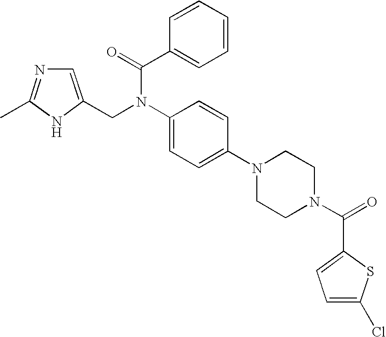

Compound 318 is prepared from 4-formyl-2-methylimidazole (26 mg; 0.233 mmol) and compound 57A (75 mg; 0.233 mmol) according to the conditions used for the preparation of Example 317.

Mass spectrum (ESI): m/z 520 (MH⁺).

### EXAMPLE 319

### N-{4-[4-(5-Chloro-2-thiophenecarbonyl)-1-piperazinyl]phenyl}-N-thiazol-2-ylmethyl-benzamide

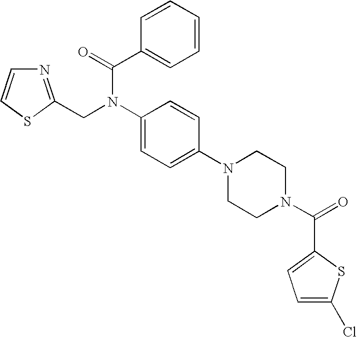

Compound 319 is prepared from 2-formylthiazole (20 μl; 0.233 mmol) and compound 57A (75 mg; 0.233 mmol) according to the conditions used for the preparation of Example 317.

Mass spectrum (ESI): m/z 523 (MH⁺).

### EXAMPLE 320

### N-{4-[4-(5-Chloro-2-thiophenecarbonyl)-1-piperazinyl]phenyl}-N-(2-phenyl-1Himidazol-4-ylmethyl)benzamide

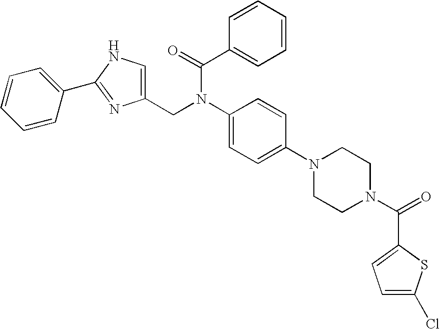

Compound 320 is prepared from 4-formyl-2-phenylimidazole (40 mg; 0.233 mmol) and compound 57A (75 mg; 0.233 mmol) according to the conditions used for the preparation of Example 317.

Mass spectrum (ESI): m/z 582 (MH⁺).

The derivatives of the present invention are inhibitors of the prenylation of proteins and more particularly of the famesylation of ras proteins, as shown by the inhibition studies on protein famesyl transferase and on protein geranylgeranyl transferase.

A) Evaluation of the Inhibition of Protein Famesyl Transferase:

Principle:

The famesylation of the dansylated peptide GCVLS, catalyzed with the enzyme protein famesyl transferase results in a change in the emission spectrum of the dansyl group, and especially an increase in the emission at 505 nm when the molecule is excited at 340 nm. When measured using the spectrofluorimeter, this emission is proportional to the activity of the enzyme (Pompliano et al., J. Am. Chem. Soc. 1992; 114: 7945-7946).

Materials

Reaction Buffer:

55 mM TRIS/HCl pH 7.5; 5.5 mM DTT; 5.5 mM MgCl₂; 110 μM ZnCl₂, 0.22% B-octyl-B D-glucopyrannoside.

Substrates:

Farnesyl pyrophosphate (FPP), (Sigma)

Dansylated peptide dansyl-GCVLS (Neosystem/Strasbourg, France)

Enzyme:

The protein farnesyl transferase is partially purified from bovine brain by ion-exchange chromatography on Q-sepharose (Pharmacia) (Moores et al., J. Biol. Chem. 1991, 266: 14603-14610, Reiss et al., Cell 1990, 62: 81-88).

Method

The reaction mixture containing 2 μM of FPP, 2 μM of dansyl GCVLS with or without (zero) the amount of enzyme giving an intensity of 100 on the spectrofluorimeter after incubation for 10 minutes at 37° C., is prepared on ice.

In an Eppendorf tube, 360 μL of reaction mixture are mixed with 40 μl of 10× concentrated test product or of solvent, and incubated for 10 minutes at 37° C. The reaction is quenched on ice and the fluorescence intensity is measured (excitation at 340 nm, 4 nm slit, emission at 505 nm, 10 nm slit). The tests are performed in duplicate. The results are expressed as a percentage of inhibition. Under these conditions, the derivatives of the present invention were identified as powerful inhibitors of protein farnesyl transferase (IC₅₀<10 μM).

Adaptation of the method to a 96-well format:

The procedure is similar to that above, except that the measurements are performed in a “Black Fluorotrack 200” 96-well device (Greiner, Poitiers, France) and the readings are performed using a “Spectrametrix Gemini” 96-well fluorimeter (Molecular Devices, Sunnyvale, Calif., USA)

B) Evaluation of the Inhibition of Geranyl Geraryl Transferase Protein I:

Materials

Reaction Buffer:

55 mM TRIS/HCl pH 7.5; 5.5 mM DTT; 5.5 mM MgCl₂; 110 μM ZnCl₂, 0.22% N-octyl-B D-glucopyrannoside.

Substrates:

³H-geranylgeranyl pyrophosphate (GGPP), 66 μM, 15 CI/mmol, (Isotopchim) Rho-GST recombinant protein

Enzyme:

GGPT I is partially purified from bovine brain by ion-exchange chromatography on Q-sepharose (Pharmacia); elution at 0.23 and 0.4 M NaCl, respectively.

(Moores et al., J. Biol. Chem. 1991, 266: 14603-14610; Reiss et al., Cell 1990, 62: 81-88).

Method

The reaction mixture containing 0.2 μM of ³H-GGPP, 1 μM of RhoA-GST with or without (zero) 5 μl of GGPT/test, is prepared on ice.

In an Eppendorf tube, 45 μl of reaction mixture are mixed with 5 μl of 10× concentrated test product or of solvent, and incubated for 45 minutes at 37° C. A 45 μl aliquot is placed on a phosphocellulose P81 filter (Whatman, Maidstone, UK) numbered, washed with 50% ethanol, phosphoric acid (0.5%) and counted by scintillation.

The tests are performed in duplicate. The results are expressed as a percentage of inhibition.

Adaptation of the Method to a 96-well Format:

The procedure is similar to that above, except that the measurements are performed in 96-well plates (Nunc, France) and the reactions are then passed through a 96-well “Unifilter” (Whatman, Maidstone, UK) containing a phosphocellulose P81 buffer using a “Filtermate 196” system (Packard, France). After washing with 50% ethanol and phosphoric acid (0.5%), the filters are counted by scintillation on a “Packard Topcount” instrument.

The tests are performed in triplicate. The results are expressed as a percentage of inhibition.

The derivatives of the present invention are inhibitors of enzymes that catalyze the prenylation of proteins and more particularly of PFTase. They are distinguished from the closest derivatives of the prior art not only by their novel chemical structure, but also by their biological activity and more particularly by their efficacy in inhibiting PFTase.

C)Results:

The compounds of the present invention described in the above examples were tested to determin their inhibitory activity on PFIase according to the above method. They were found to inihibit PFase with an IC₅₀ value <1 μM.

The few examples that follow, chosen from the compounds of the present invention, illustrate entirely unexpected capacity of the compounds to display powerful inhibition of PFTase and occasionally selective inhibition relative to PGGTase:

| Example | IC₅₀ (PFTase, nM) | IC₅₀ (PGGTase, nM) |
|---|---|---|
| 45 | 6 | 10000 |
| 82 | 7 | 10000 |
| 108 | 2 | — |
| 109 | 1 | 700 |
| 110 | 2 | — |
| 113 | 2 | — |
| 114 | 2 | 10000 |
| 123 | 2 | 10000 |
| 124 | 2 | — |
| 126 | 0.9 | 10000 |
| 141 | 9 | — |

Pharmaceutical compositions containing, as active ingredients, a compound of general formula (I) or a physiologically acceptable salt of a compound of general Formula (I) combined with one or more therapeutic agents such as, for example, anticancer agents such as, for example, cytotoxic anticancer agents such as navelbine, vinflunine, taxol, taxotere, 5-fluorouracil, methotrexate, doxorubicin, camptothecin, gemcitabin, etoposide, cisplatin or BCNU, or hormonal anticancer agents, for instance tamoxifen or medroxyprogesterone, should also be considered as forming part of the present invention. Alternatively, in combination with an inhibitor of the biosynthesis of farnesyl and geranylgeranyl pyrophosphates, such as an inhibitor of HMG-CoA reductase, for instance lovastatin, simvastatin, pravastatin, fluvastatin, atorvastatin or cerivastatin. Treatment with radiation (X-rays or gamma rays), which may be delivered using an external source or by implanting minute internal radioactive sources, may also be combined with the administration of an inhibitor of protein farnesyl transferase belonging to the present invention. These treatments may be used for treating or preventing cancers, such as cancer of the lungs, of the pancreas, of the skin, of the head, of the neck, of the uterus, or of the ovaries, anal cancer, cancer of the stomach, of the colon, of the breast, of the esophagus, of the small intestine, of the thyroid gland, of the prostate, of the kidney or of the bladder, acute or chronic leukemias, or alternatively a combination or 2 or more of these cancers. These treatments may also be used for treating or preventing restenosis or atherosclerosis, infections associated with PFTase such as delta hepatitis, or benign proliferative disorders.

A subject of the present invention is also pharmaceutical compositions containing as active principle a compound of general formula (I) or a pharmaceutically acceptable salt thereof, mixed or combined with a suitable excipient. These compositions may be, for example, in the form of solid or liquid compositions, emulsions, lotions or creams.

Solid compositions for oral administration that may be used include tablets, pills, powders (gelatin capsules or wafer capsules) or granules. In these compositions, the active principle according to the invention is mixed with one or more inert diluents such as starch, cellulose, sucrose, lactose or silica, under a stream of argon. These compositions may also comprise substances other than diluents, for example one or more lubricants such as magnesium stearate or talc, a colorant, a coating (dragees) or a varnish.

Liquid compositions for oral administration that may be used include pharmaceutically acceptable solutions, suspensions, emulsions, syrups and elixirs containing inert diluents such as water, ethanol, glycerol, plant oils or liquid paraffin. These compositions may comprise substances other than diluents, for example wetting, sweetening, thickening, flavoring or stabilizing products.

The sterile compositions for parenteral administration may preferably be aqueous or nonaqueous solutions, suspensions or emulsions.

Solvents or vehicles that may be used include water, propylene glycol, a polyethylene glycol, plant oils, in particular olive oil, and injectable organic esters, for example ethyl oleate, or other suitable organic solvents. These compositions may also contain adjuvants, in particular wetting agents, isotonic agents, emulsifiers, dispersants and stabilizers. The sterilization may be performed in several ways, for example by aseptic filtration, by incorporating sterilizing agents into the composition, by irradiation or by heating. They may also be prepared in the form of sterile solid compositions that may be dissolved at the time use in sterile water or any other injectable sterile medium.

The compositions for rectal administration are suppositories or rectal capsules containing, in addition to the active product, excipients such as cocoa butter, semisynthetic glycerides or polyethylene glycols.

The compositions for topical administration may be, for example, creams, lotions, eyedrops, mouth washes, nasal drops or aerosols.

The doses depend on the desired effect, the duration of the treatment and the administration route used; they are generally between 0.001 g and 1 g (preferably between 0.005 g and 0.75 g) per day, preferably orally, for an adult, with unit doses ranging from 0.1 mg to 500 mg of active substance.

In general, the doctor will determine the appropriate dosage as a function of the age and weight and all the other personal factors of the individual to be treated.

1. A compound corresponding to the general formula (I): 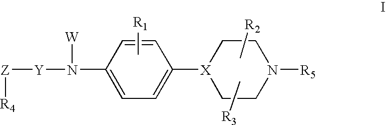
in which
W represents:
hydrogen, COR₆, CSR₆, SO₂R₆, CO(CH₂)₆R₆, (CH₂)ₙR₇
X represents:
CH or N.
Y represents:
(CH₂)ₙ, CO, CH₂CO, CH═CHCO, CH₂CH₂CO.
when Y=CO, CH₂CO, CH═CH—CO or CH₂CH₂—CO then W represents only hydrogen,
Z represents:
imidazole, benzimidazole, isoxazole, tetrazole, oxadiazole, thiazole, thiadiazole, pyridine, quinazoline, quinoxaline. These heterocycles may be unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH, CONHOH, SO₂NH₂, CONH₂.
When Z=pyridine, then Y is other than CO.
R₁ represents:
hydrogen, C₁-C₆ alkyl, halogen, OCH₃, CF₃
R₂ and R₃, which may be identical or different, represent:
hydrogen, C₁-C₆alkyl.
R₄ represents:
a) hydrogen,
b) C₁-C₆ alkyl which is unsubstituted or substituted with an aryl, a cyanophenyl, a nitrophenyl, an aminophenyl, a methoxyphenyl, a hydroxyphenyl, a heterocycle, a halogen, CN, NO₂, OR₂, SR₂, NR₂R₃ COOR₂.
c) an aryl,
d) a heterocycle,
R₅ represents:
hydrogen, COR₇, SO₂R₇, CO(CH₂)ₙSR₇, CO(CH₂)ₙOR₇, CONR₇R₈, CSNR₇R₈, CO(CH₂)ₘCOR₇.
R₆ represents:
a) a phenyl or a naphthyl which is unsubstituted or substituted with one or more residues chosen from C₁-C₆ alkyl, halogen, phenyl, naphthyl, NO₂, CN, CF₃, OR₇, SR₇, NR₇R₈, COOR₇, CONR₇R₈, COR₇,
b) a C₁-C₆ alkyl, a cycloalkyl,
c) a heterocycle.
d) NR₇R₈,
R₇ and R₈, which may be identical or different, represent:
a) hydrogen; C₁-C₁₅ alkyl, which is unsubstituted or substituted with a halogen, COOMe, COOH, OMe, OH, CF₃, CN, SMe; a cycloalkyl which is unsubstituted or substituted with a halogen, OMe, OH, CF₃, CN, SMe; an alkylcycloalkyl which is unsubstituted or substituted with a halogen, OMe, OH, CF₃, CN, SMe; or R₇ and R₈ when they are adjacent, taken together, can form a 4- to 6-membered ring with the nitrogen atom to which they are attached,
b) an heterocycle, an alkylheterocycle,
c) an aryl, an alkylaryl,
n represents:
0 to 10
m represents:
2 to 10
on condition, however, that Z represents a quinazoline or benzimidazole group, then R₅ is other than CH₂Ph or methyl and n is other than zero,
and also the therapeutically acceptable salts and solvates thereof, the abovementioned compound possibly being in the form of pure optical isomers or mixtures of optical isomers in all proportions, including the racemic form.

2. The compound as claimed in claim 1, characterized in that R₁, R₂, R₃ and R₄ each represent a hydrogen and Y is a methylene (CH₂).

3. The compound as claimed in either of claims 1 and 2, characterized in that Z represents an imidazolyl or pyridyl residue.

4. The compound as claimed in claim 3, characterized in that Z represents an imidazolyl residue and R₄ is a benzyl group substituted with a nitrile, nitro or methoxy group in position 4.

5. The compound as claimed in one of claims 1 to 4, characterized in that X represents a nitrogen atom.

6. The compound as claimed in one of claims 1 to 4, characterized in that X represents a carbon atom.

7. The compound as claimed in one of claims 1 to 6, characterized in that R₅ represents 2-thiophenecarbonyl or 5-chloro-2-thiophenecarbonyl.

8. The compound as claimed in one of claims 1 to 6, characterized in that R₅ represents CSNR₇R₈

9. The compound as claimed in one of claims 1 to 8, characterized in that W represents COR₆.

10. The compound as claimed in one of claims 1 to 8, characterized in that W represents (CH₂)ₙR₇.

11. The compound as claimed in claim 1, selected from:
N-(3H-imidazol-4-ylmethyl)-4-nitro-N-{4-[4-(thiophene-2-carbonyl)-1-piperazinyl]phenyl}benzamide
4-{4-[benzenesulfonyl-(3H-imidazol-4-ylmethyl)amino]phenyl}-1-piperazinecarbothioic acid propylamide
4-{4-[benzenesulfonyl-(3H-imidazol-4-ylmethyl)amino]phenyl}-1-piperazinecarbothioic acid cyclohexylamide
4-{4-[benzenesulfonyl-(3H-imidazol-4-ylmethyl)amino]phenyl}-1-piperazinecarbothioic acid cyclohexylmethyl-amide
4-{4-[benzenesulfonyl-(3H-imidazol-4-ylmethyl)amino]phenyl}-1-piperazinecarbothioic acid benzylamide
4-{4-[benzenesulfonyl-(3H-imidazol-4-ylmethyl)amino]phenyl}-1-piperazinecarbothioic acid isobutylamide
4-{4-[benzenesulfonyl-(3H-imidazol-4-ylmethyl)amino]phenyl}-1-piperazinecarbothioic acid (2-methoxyethyl)amide
N-{4-[4-(5-chloro-2-thiophenecarbonyl)-1-piperazinyl]phenyl}-3-fluoro-N-(3H-imidazol-4-ylmethyl)benzamide
4-[5-({4-[4-(5-chloro-2-thiophenecarbonyl)-1-piperazinyl]phenylamino}methyl)-imidazol-1-ylmethyl]benzonitrile
N-{4-[4-(5-chloro-2-thiophenecarbonyl)-1-piperazinyl]phenyl}-N-[3-(4-cyano-benzyl)-3H-imidazol-4-ylmethyl]benzamide
N-{4-[4-(5-chloro-2-thiophenecarbonyl)-1-piperazinyl]phenyl}-N-[3-(4-cyano-benzyl)-3H-imidazol-4-ylmethyl]benzenesulfonamide
N-{4-[4-(5-chloro-2-thiophenecarbonyl)-1-piperazinyl]phenyl}-4-cyano-N-(3H-imidazol-4-ylmethyl)benzamide thiophene-2-carboxylic acid {4-[4-(5-chloro-2-thiophenecarbonyl)-1-piperazinyl]phenyl}[3-(4-cyanobenzyl)-3H-imidazol-4-ylmethyl]amide
2-chloro-N-{4-[4-(5-chloro-2-thiophenecarbonyl)-1-piperazinyl]phenyl}-N-[3-(4-cyanobenzyl)-3H-imidazol-4-ylmethyl]benzamide
3-chloro-N-{4-[4-(5-chloro-2-thiophenecarbonyl)-1-piperazinyl]phenyl}-N-[3-(4-cyanobenzyl)-3H-imidazol-4-ylmethyl]benzamide
4-chloro-N-{4-[4-(5-chloro-2-thiophenecarbonyl)-1-piperazinyl]phenyl}-N-[3-(4-cyanobenzyl)-3H-imidazol-4-ylmethyl]benzamide
N-{4-[4-(5-chloro-2-thiophenecarbonyl)-1-piperazinyl]phenyl}-N-[3-(4-cyano-benzyl)-3H-imidazol-4-ylmethyl]-2-fluorobenzamide
N-{4-[4-(5-chloro-2-thiophenecarbonyl)-1-piperazinyl]phenyl}-N-[3-(4-cyano-benzyl)-3H-imidazol-4-ylmethyl]-3-fluorobenzamide
N-{4-[4-(5-chloro-2-thiophenecarbonyl)-1-piperazinyl]phenyl}-N-[3-(4-cyano-benzyl)-3H-imidazol-4-ylmethyl]-4-fluorobenzamide
N-{4-[4-(5-chloro-2-thiophenecarbonyl)-1-piperazinyl]phenyl}-N-[3-(4-cyanobenzyl)-3H-imidazol-4-ylmethyl]-2-trifluoromethylbenzamide
N-{4-[4-(5-chloro-2-thiophenecarbonyl)-1-piperazinyl]phenyl}-N-[3-(4-cyanobenzyl)-3H-imidazol-4-ylmethyl]-2-methoxybenzamide
N-{4-[4-(5-chloro-2-thiophenecarbonyl)-1-piperazinyl]phenyl}-N-[3-(4-cyanobenzyl)-3H-imidazol-4-ylmethyl]-3-methoxybenzamide
N-{4-[4-(5-chloro-2-thiophenecarbonyl)-1-piperazinyl]phenyl}-N-[3-(4-cyanobenzyl)-3H-imidazol-4-ylmethyl]-4-methoxybenzamide
N-{4-[4-(5-chloro-2-thiophenecarbonyl)-1-piperazinyl]phenyl}-N-[3-(4-cyanobenzyl)-3H-imidazol-4-ylmethyl]-2-nitrobenzamide
N-{4-[4-(5-chloro-2-thiophenecarbonyl)-1-piperazinyl]phenyl}-N-[3-(4-cyanobenzyl)-3H-imidazol-4-ylmethyl]-3-nitrobenzamide
N-{4-[4-(5-chloro-2-thiophenecarbonyl)-1-piperazinyl]phenyl}-N-[3-(4-cyanobenzyl)-3H-imidazol-4-ylmethyl]-4-nitrobenzamide
N-{4-[4-(5-chloro-2-thiophenecarbonyl)-1-piperazinyl]phenyl}-4-cyano-N-[3-(4-cyanobenzyl)-3H-imidazol-4-ylmethyl]-benzamide cyclohexanecarboxylic acid {4-[4-(5-chloro-thiophene-2-carbonyl)-1-piperazinyl]phenyl}-[3-(4-cyanobenzyl)-3H-imidazol-4-ylmethyl]-amide
N-{4-[4-(5-chloro-2-thiophenecarbonyl)-1-piperazinyl]phenyl}-2-cyano-N-[3-(4-cyanobenzyl)-3H-imidazol-4-ylmethyl]-benzenesulfonamide
N-{4-[4-(5-chloro-2-thiophenecarbonyl)-1-piperazinyl]phenyl}-3-cyano-N-[3-(4-cyanobenzyl)-3H-imidazol-4-ylmethyl]benzenesulfonamide
N-{4-[4-(5-chloro-2-thiophenecarbonyl)-1-piperazinyl]phenyl}-4-cyano-N-[3-(4-cyanobenzyl)-3H-imidazol-4-ylmethyl]benzenesulfonamide
1-{4-[4-(5-chloro-2-thiophenecarbonyl)-1-piperazinyl]phenyl}-1-(3H-imidazol-4-ylmethyl)-3-propylurea
1-{4-[4-(5-chloro-2-thiophenecarbonyl)-1-piperazinyl]phenyl}-1-(3H-imidazol-4-ylmethyl)-3-(2-methylsulfanylphenyl)urea
N-(3H-imidazol4-ylmethyl)-4-nitro-N-[4-(4-propylthiocarbamoyl-1-piperazinyl)phenyl]benzamide
N-[4-(4-cyclohexylthiocarbamoyl-1-piperazinyl)phenyl]-N-(3H-imidazol-4-ylmethyl)-4-nitrobenzamide
N-(3H-imidazol-4-ylmethyl)-N-[4-(4-isobutylthiocarbamoyl-1-piperazinyl)phenyl]-4-nitrobenzamide
4-{4-[benzenesulfonyl-(3H-imidazol-4-ylmethyl)amino]phenyl}piperidine-1-carbothioic acid isobutylamide
N-(3H-imidazol-4-ylmethyl)-N-[4-(1-isobutylthiocarbamoylpiperidin-4-yl)phenyl]-4-nitrobenzamide cyclohexanecarboxylic acid (3H-imidazol-4-ylmethyl)-[4-(1-isobutylthiocarbamoyl-piperidin-4-yl)-phenyl]amide
4-{4-[(4-cyano-benzenesulfonyl)(3H-imidazol-4-ylmethyl)amino]phenyl}piperidine-1-carbothioic acid cyclohexylamide
N-{4-[4-(5-chloro-2-thiophenecarbonyl)-1-piperazinyl]phenyl}-3-(3H-imidazol-4-yl)acryamide
N-{4-[4-(5-chloro-2-thiophenecarbonyl)-1-piperazinyl]phenyl}-3-[3-(4-cyano-benzyl)-3H-imidazol-4-yl]propionamide
4-(5-{[{4-[4-(5-chloro-2-thiophenecarbonyl)-1-piperazinyl]phenyl}(3-fluoro-benzyl)amino]methyl}imidazol-1-ylmethyl)benzonitrile
4-(5-{[{4-[4-(5-chloro-2-thiophenecarbonyl)-1-piperazinyl]phenyl}(4-fluoro-benzyl)amino]methyl}imidazol-1-ylmethyl)benzonitrile
4-(5-{[{4-[4-(5-chloro-2-thiophenecarbonyl)-1-piperazinyl]phenyl}(3-hydroxy-benzyl)amino]methyl}imidazol-1-ylmethyl)-benzonitrile
4-(5-{[{4-[4-(5-chloro-2-thiophenecarbonyl)-1-piperazinyl]phenyl}(3-methoxyl-benzyl)amino]methyl}imidazol-1-ylmethyl)benzonitrile
4-(5-{[{4-[4-(5-chloro-2-thiophenecarbonyl)-1-piperazinyl]phenyl}-(3,4-dichloro-benzyl)amino]methyl}imidazol-1-ylmethyl)benzonitrile
4-{5-[({4-[4-(5-chloro-2-thiophenecarbonyl)-1-piperazinyl]phenyl}thiophen-3-ylmethylamino)methyl]imidazol-1-ylmethyl}benzonitrile
4-{5-[(benzo[1,3]dioxol-5-ylmethyl-{4-[4-(5-chloro-2-thiophenecarbonyl)-1-piperazinyl]phenyl}amino)methyl]imidazol-1-ylmethyl}benzonitrile
4-{5-[(butyl-{4-[4-(5-chloro-2-thiophenecarbonyl)-1-piperazinyl]phenyl}amino)-methyl]imidazol-1-ylmethyl}benzonitrile
4-(5-{[{4-[4-(5-chloro-2-thiophenecarbonyl)-1-piperazinyl]phenyl}(3-phenyl-propyl)amino]methyl}imidazol-1-ylmethyl)benzonitrile
4-{5-[((3-fluorobenzyl)-{4-[4-(thiophene-2-carbonyl)-1-piperazinyl]phenyl}amino)-methyl]imidazol-1-ylmethyl}benzonitrile
4-{5-[((4-fluorobenzyl)-{4-[4-(thiophene-2-carbonyl)-1-piperazinyl]phenyl}amino)-methyl]imidazol-1-ylmethyl}benzonitrile
4-{5-[((4-fluorobenzyl)-{4-[4-(thiophene-2-carbonyl)-1-piperazinyl]phenyl}amino)-methyl]imidazol-1-ylmethyl}benzonitrile
4-{5-[((3-methoxybenzyl)-{4-[4-(thiophene-2-carbonyl)-1-piperazinyl]phenyl}-amino)methyl9 imidazol-1-ylmethyl}benzonitrile
4-{5-[((3,4-dichlorobenzyl)-{4-[4-(thiophene-2-carbonyl)-1-piperazinyl]phenyl}-amino)methyl]imidazol-1-ylmethyl}benzonitrile
4-{5-[({4-[4-(thiophene-2-carbonyl)-1-piperazinyl]phenyl}thiophen-3-ylmethyl-amino)methyl]imidazol-1-ylmethyl}benzonitrile
4-{5-[(benzo[1,3]dioxol-5-ylmethyl-{4-[4-(thiophene-2-carbonyl)-1-piperazinyl}amino)methyl]imidazol-1-ylmethyl}benzonitrile
4-{5-[(butyl-{4-[4-(thiophene-2-carbonyl)-1-piperazinyl]phenyl}amino)methyl]-imidazol-1-ylmethyl}benzonitrile
4-{5-[((3-phenylpropyl)-{4-[4-(thiophene-2-carbonyl)-1-piperazinyl]phenyl}-amino)methyl]imidazol-1-ylmethyl}benzonitrile
N-{4-[4-(5-chloro-2-thiophenecarbonyl)-1-piperazinyl]phenyl}-N-(1-methyl-1H-benzoimidazol-2-ylmethyl)benzamide
N-{4-[4-(5-chloro-2-thiophenecarbonyl)-1-piperazinyl]phenyl}-N-(2-methyl-3H-imidazol-4-ylmethyl)benzamide
N-{4-[4-(5-chloro-2-thiophenecarbonyl)-1-piperazinyl]phenyl}-N-thiazol-2-ylmethylbenzamide
N-{4-[4-(3-chloro-2-thiophenecarbonyl)-1-piperazinyl]phenyl}-N-[3-(4-cyanobenzyl)-3H-imidazol-4-ylmethyl]benzamide
N-{4-[4-(benzo[b]thiophene-2-carbonyl)-1-piperazinyl]phenyl}-N-[3-(4-cyanobenzyl)-3H-imidazol-4-ylmethyl]benzamide
N-{4-[4-(3-chlorobenzo[b]thiophene-2-carbonyl)-1-piperazinyl]phenyl}-N-[3-(4-cyanobenzyl)-3H-imidazol-4-ylmethyl]benzamide
N-[3-(4-cyanobenzyl)-3H-imidazol-4-ylmethyl]-N-{4-[4-(2-thiophen-2-ylacetyl)-1-piperazinyl]phenyl}benzamide
N-[3-(4-cyanobenzyl)-3H-imidazol-4-ylmethyl]-N-{4-[4-(furan-2-carbonyl)-1-piperazinyl]phenyl}benzamide
N-[3-(4-cyanobenzyl)-3H-imidazol-4-ylmethyl]-N-{4-[4-(isoxazole-5-carbonyl)-1-piperazinyl]phenyl}benzamide
N-(4-{4-[3-(2-chlorophenyl)-5-methylisoxazole-4-carbonyl]-1-piperazinyl}phenyl)-N-[3-(4-cyanobenzyl)-3H-imidazol-4-ylmethyl]benzamide
N-[3-(4-cyanobenzyl)-3H-imidazol-4-ylmethyl]-N-{4-[4-(5-methylthiophene-2-carbonyl)-1-piperazinyl]phenyl}benzamide
N-{4-[4-(5-bromothiophene-2-carbonyl)-1-piperazinyl]phenyl}-N-[3-(4-cyanobenzyl)-3H-imidazol-4-ylmethyl]benzamide
N-[3-(4-cyanobenzyl)-3H-imidazol-4-ylmethyl]-N-{4-[4-(thiophene-3-carbonyl)-1-piperazinyl]phenyl}benzamide
N-[3-(4-cyanobenzyl)-3H-imidazol-4-ylmethyl]-N-{4-[4-(2-thiophen-3-ylacetyl)-1-piperazinyl]phenyl}benzamide
N-[3-(4-cyanobenzyl)-3H-imidazol-4-ylmethyl]-N-{4-[4-(4-thiophen-2-ylbutyryl)-1-piperazinyl]phenyl}benzamide
N-(4-{4-[2-(5-chlorobenzo[b]thiophen-3-yl)acetyl]-1-piperazinyl}phenyl)-N-[3-(4-cyanobenzyl)-3H-imidazol-4-ylmethyl]benzamide
N-[3-(4-cyanobenzyl)-3H-imidazol-4-ylmethyl]-N-{4-[4-(4-oxo4-thiophen-2-ylbutyryl)-1-piperazinyl]phenyl}benzamide
N-[3-(4-cyanobenzyl)-3H-imidazol-4-ylmethyl]-N-{4-[4-(5-oxo-5-thiophen-2-ylpentanoyl)-1-piperazinyl]phenyl}benzamide
N-[3-(4-cyanobenzyl)-3H-imidazol-4-ylmethyl]-N-{4-[4-(1-methyl-1H-pyrrole-2-carbonyl)-1-piperazinyl]phenyl}benzamide
N-(4-{4-[5-(4-chlorophenyl)-furan-2-carbonyl]-1-piperazinyl}-phenyl)-N-[3-(4-cyanobenzyl)-3H-imidazol-4-ylmethyl]benzamide
N-[3-(4-cyanobenzyl)-3H-imidazol-4-ylmethyl]-N-{4-[4-(5-methyl-1-phenyl-1H-pyrazole-4-carbonyl)-1-piperazinyl]phenyl}benzamide
N-[3-(4-cyanobenzyl)-3H-imidazol-4-ylmethyl]-N-{4-[4-(3,5-dimethylisoxazole-4-carbonyl)-1-piperazinyl]phenyl}benzamide
N-[3-(4-cyanobenzyl)-3H-imidazol-4-ylmethyl]-N-{4-[4-(5-methyl-2-phenyl-2H-[1,2,3]triazole-4-carbonyl)-1-piperazinyl]phenyl}benzamide
N-{4-[4-(5-chlorothiophen-2-ylmethyl)-1-piperazinyl]phenyl}-N-[3-(4-cyanobenzyl)-3H-imidazol-4-ylmethyl]benzamide
N-[3-(4-cyanobenzyl)-3H-imidazol-4-ylmethyl]-N-{4-[4-(3-methylthiophen-2-ylmethyl)-1-piperazinyl]phenyl}benzamide
N-[3-(4-cyanobenzyl)-3H-imidazol-4-ylmethyl]-N-{4-[4-(5-methylthiophen-2-ylmethyl)-1-piperazinyl]phenyl}benzamide
N-[3-(4-cyanobenzyl)-3H-imidazol-4-ylmethyl]-N-{4-[4-(5-ethylthiophen-2-ylmethyl)-1-piperazinyl]phenyl}benzamide
N-{4-[4-(5-chloro-2-thiophenecarbonyl)-1-piperazinyl]phenyl}-N-(2-phenyl-1H-imidazol-4-ylmethyl)benzamide
4-(4-{benzenesulfonyl-[3-(4-cyanobenzyl)-3H-imidazol-4-ylmethyl]amino}phenyl)-1-piperazinecarbothioic acid isobutylamide
4-(4-{benzenesulfonyl-[3-(4-cyanobenzyl)-3H-imidazol-4-ylmethyl]amino}phenyl)-1-piperazinecarbothioic acid propylamide
4-(4-{benzenesulfonyl-[3-(4-cyanobenzyl)-3H-imidazol-4-ylmethyl]amino}phenyl)-1-piperazinecarbothioic acid cyclohexylamide
4-(4-{benzenesulfonyl-[3-(4-cyanobenzyl)-3H-imidazol-4-ylmethyl]amino}phenyl)-1-piperazinecarbothioic acid benzylamide
4-(4-{(4-cyanobenzenesulfonyl)-[3-(4-cyanobenzyl)-3H-imidazol-4-ylmethyl]-amino}phenyl)-1-piperazinecarbothioic acid isobutylamide
4-(4-{(4-cyanobenzenesulfonyl)-[3-(4-cyanobenzyl)-3H-imidazol-4-ylmethyl]-amino}phenyl)-1-piperazinecarbothioic acid propylamide
4-(4-{(4-cyanobenzenesulfonyl)-[3-(4-cyanobenzyl)-3H-imidazol-4-ylmethyl]-amino}phenyl)-1-piperazinecarbothioic acid cyclohexylamide
4-(4-{(4-cyanobenzenesulfonyl)-[3-(4-cyanobenzyl)-3H-imidazol-4-ylmethyl]-amino}phenyl)-1-piperazinecarbothioic acid cyclohexylmethylamide
4-(4-{(4-cyanobenzenesulfonyl)-[3-(4-cyanobenzyl)-3H-imidazol-4-ylmethyl]-amino}phenyl)-1-piperazinecarbothioic acid benzylamide
4-cyano-N-[3-(4-cyanobenzyl)-3H-imidazol-4-ylmethyl]-N-[4-(4-isobutylthiocarbamoyl-1-piperazinyl)phenyl]benzamide
4-cyano-N-[3-(4-cyanobenzyl)-3H-imidazol-4-ylmethyl]-N-[4-(4-propylthiocarbamoyl-1-piperazinyl)phenyl]benzamide
4-cyano-N-[3-(4-cyanobenzyl)-3H-imidazol-4-ylmethyl]-N-[4-(4-cyclohexylthiocarbamoyl-1-piperazinyl)phenyl]benzamide
4-cyano-N-[3-(4-cyanobenzyl)-3H-imidazol-4-ylmethyl]-N-{4-[4-(cyclohexylmethylthiocarbamoyl)-1-piperazinyl]phenyl}benzamide
N-[4-(4-benzylthiocarbamoyl-1-piperazinyl)-phenyl]-4-cyano-N-[3-(4-cyanobenzyl)-3H-imidazol-4-ylmethyl]benzamide
N-[3-(4-cyano-benzyl)-3H-imidazol-4-ylmethyl]-3-fluoro-N-[4-(4-isobutylthiocarbamoyl-1-piperazinyl)-phenyl]benzamide
N-[3-(4-cyanobenzyl)-3H-imidazol-4-ylmethyl]-3-fluoro-N-[4-(4-propylthiocarbamoyl-1-piperazinyl)phenyl]benzamide
N-[3-(4-cyanobenzyl)-3H-imidazol-4-ylmethyl]-N-[4-(4-cyclohexylthiocarbamoyl1-piperazinyl)phenyl]-3-fluorobenzamide
N-[4-(4-benzylthiocarbamoyl-1-piperazinyl)phenyl]-N-[3-(4-cyanobenzyl)-3H-imidazol-4-ylmethyl]-3-fluorobenzamide
4-{4-[1-[3-(4-cyanobenzyl)-3H-imidazol-4-ylmethyl]-3-(2-methylsulfanylphenyl)-ureido]phenyl}-1-piperazinecarbothioic acid propylamide
4-(4-{1-[3-(4-cyanobenzyl)-3H-imidazol-4-ylmethyl]-3-propylureido}phenyl)-1-piperazinecarbothioic acid isobutylamide
4-(4-{1-[3-(4-cyanobenzyl)-3H-imidazol-4-ylmethyl]-3-propylureido}phenyl)-1-piperazinecarbothioic acid propylamide
4-(4-{1-[3-(4-cyanobenzyl)-3H-imidazol-4-ylmethyl]-3-propylureido}phenyl)-1-piperazinecarbothioic acid cyclohexylamide
4-(4-{1-[3-(4-cyanobenzyl)-3H-imidazol-4-ylmethyl]-3-propylureido}phenyl)-1-piperazinecarbothioic acid cyclohexylmethylamide
4-(4-{1-[3-(4-cyanobenzyl)-3H-imidazol-4-ylmethyl]-3-propylureido}phenyl)-1-piperazinecarbothioic acid benzylarnide
N-(3H-imidazol-4-ylmethyl)-N-[4-(4-isobutylthiocarbamoyl-1-piperazinyl)phenyl]-3-methoxybenzamide
3-fluoro-N-(3H-imidazol-4-ylmethyl)-N-[4-(4-isobutylthiocarbamoyl-1-piperazinyl)phenyl]benzamide cyclopropanecarboxylic acid {4-[4-(5-chloro-2-thiophenecarbonyl)-1-piperazinyl]phenyl}-[3-(4-cyanobenzyl)-3H-imidazol-4-ylmethyl]amide cyclobutanecarboxylic acid {4-[4-(5-chloro-2-thiophenecarbonyl)-1-piperazinyl]phenyl}-[3-(4-cyanobenzyl)-3H-imidazol-4-ylmethyl]amide cyclopentanecarboxylic acid {4-[4-(5-chloro-2-thiophenecarbonyl)-1-piperazinyl]phenyl}-[3-(4-cyanobenzyl)-3H-imidazol-4-ylmethyl]amide
N-{4-[4-(5-chloro-2-thiophenecarbonyl)-1-piperazinyl]phenyl}-N-[3-(4-cyanobenzyl)-3H-imidazol-4-ylmethyl]-3-cyclopentylpropionamide
N-{4-[4-(5-chloro-2-thiophenecarbonyl)-1-piperazinyl]phenyl}-N-[3-(4-cyanobenzyl)-3H-imidazol-4-ylmethyl]-2-cyclopentylacetamide naphthalene-1-carboxylic acid {4-[4-(5-chloro-2-thiophenecarbonyl)-1-piperazinyl]phenyl]}-[3-(4-cyanobenzyl)-3H-imidazol-4-ylmethyl]amide biphenyl-4-carboxylic acid {4-[4-(5-chloro-2-thiophenecarbonyl)-1-piperazinyl]phenyl}-[3-(4-cyanobenzyl)-3H-imidazol-4-ylmethyl]amide
N-{4-[4-(5-chloro-2-thiophenecarbonyl)-1-piperazinyl]phenyl}-N-[3-(4-cyanobenzyl)-3H-imidazol-4-ylmethyl]-3,5-dimethoxybenzamide
N-{4-[4-(5-chloro-2-thiophenecarbonyl)-1-piperazinyl]phenyl}-N-[3-(4-cyanobenzyl)-3H-imidazol-4-ylmethyl]-3-methylbenzamide
N-{4-[4-(5-chloro-2-thiophenecarbonyl)-1-piperazinyl]phenyl}-N-[3-(4-cyanobenzyl)-3H-imidazol-4-ylmethyl]-4-ethoxybenzamide
N-{4-[4-(5-chloro-2-thiophenecarbonyl)-1-piperazinyl]phenyl}-N-[3-(4cyanobenzyl)-3H-imidazol-4-ylmethyl]-4-ethylbenzamide
N-{4-[4-(5-chloro-2-thiophenecarbonyl)-1-piperazinyl]phenyl}-N-[3-(4cyanobenzyl)-3H-imidazol-4-ylmethyl]-4-hexylbenzamide
N-{4-[4-(5-chloro-2-thiophenecarbonyl)-1-piperazinyl]phenyl}-N-[3-(4-cyanobenzyl)-3H-imidazol-4-ylmethyl-4-methylbenzamide
4-butyl-N-{4-[4-(5-chloro-2-thiophenecarbonyl)-1-piperazinyl]phenyl}-N-[3-(4-cyanobenzyl)-3H-imidazol-4-ylmethyl]benzamide
N-{4-[4-(5-chloro-2-thiophenecarbonyl)-1-piperazinyl]phenyl}-N-[3-(4-cyanobenzyl)-3H-imidazol-4-ylmethyl]-3,4-difluorobenzamide
N-{4-[4-(5-chloro-2-thiophenecarbonyl)-1-piperazinyl]phenyl}-N-[3-(4-cyanobenzyl)-3H-imidazol-4-ylmethyl]-3-trifluoromethoxybenzamide
N-{4-[4-(5-chloro-2-thiophenecarbonyl)-1-piperazinyl]phenyl}-N-[3-(4-cyanobenzyl)-3H-imidazol-4-ylmethyl]acetamide
N-{4-[4-(5-chloro-2-thiophenecarbonyl)-1-piperazinyl]phenyl}-N-[3-(4-cyanobenzyl)-3H-imidazol-4-ylmethyl]-2-phenoxyacetamide
N-{4-[4-(5-chloro-2-thiophenecarbonyl)-1-piperazinyl]phenyl}-N-[3-(4-cyanobenzyl)-3H-imidazol-4-ylmethyl]-2-phenylacetamide
N-{4-[4-(5-chloro-2-thiophenecarbonyl)-1-piperazinyl]phenyl}-N-[3-(4-cyanobenzyl)-3H-imidazol-4-ylmethyl]propinamide
N-{4-[4-(5-chloro-2-thiophenecarbonyl)-1-piperazinyl]phenyl}-N-[3-(4-cyanobenzyl)-3H-imidazol-4-ylmethyl]butyramide furan-2-carboxylic acid {4-[4-(5-chloro-2-thiophenecarbonyl)-1-piperazinyl]phenyl}-[3-(4-cyanobenzyl)-3H-imidazol-4-ylmethyl]amide
N-{4-[4-(5-chloro-2-thiophenecarbonyl)-1-piperazinyl]phenyl}-N-[3-(4-cyanobenzyl)-3H-imidazol-4-ylmethyl]-2-thiophen-2-ylacetamide
N-{4-[4-(5-chloro-2-thiophenecarbonyl)-1-piperazinyl]phenyl}-N-[3-(4-cyanobenzyl)-3H-imidazol-4-ylmethyl]-methanesulfonamide
N-{4-[4-(5-chloro-2-thiophenecarbonyl)-1-piperazinyl]phenyl}-N-[3-(4-cyanobenzyl)-3H-imidazol-4-ylmethyl]-3,4-dimethylbenzamide
N-{4-[4-(5-chloro-2-thiophenecarbonyl)-1-piperazinyl]phenyl}-N-[3-(4-cyanobenzyl)-3H-imidazol-4-ylmethyl]-3-fluoro-4-methylbenzamide
2-chloro-N-{4-[4-(5-chloro-2-thiophenecarbonyl)-1-piperazinyl]phenyl}-N-[3-(4-cyanobenzyl)-3H-imidazol-4-ylmethyl]-5-methylsulfanylbenzamide
3-methoxycyclohexanecarboxylic acid {4-[4-(5-chloro-2-thiophenecarbonyl)-1-piperazinyl]phenyl}[3-(4-cyanobenzyl)-3H-imidazol-4-ylmethyl]amide
4-methylcyclohexanecarboxylic acid {4-[4-(5-chloro-2-thiophenecarbonyl)-1-piperazinyl]phenyl}[3-(4-cyanobenzyl)-3H-imidazol-4-ylmethyl]amide
1-methylcyclohexanecarboxylic acid {4-[4-(5-chloro-2-thiophenecarbonyl)-1-piperazinyl]phenyl}-[3-(4-cyanobenzyl)-3H-imidazol-4-ylmethyl]amide
N-{4-[4-(5-chloro-2-thiophenecarbonyl)-1-piperazinyl]phenyl}-N-[3-(4-cyanobenzyl)-3H-imidazol-4-ylmethyl]-2-naphthalen-1-ylacetamide
2-phenylcyclopropanecarboxylic acid {4-[4-(5-chloro-2-thiophenecarbonyl)-1-piperazinyl]phenyl}[3-(4-cyanobenzyl)-3H-imidazol-4-ylmethyl]amide
4-{5-[(benzyl-{4-[4-(5-chloro-2-thiophenecarbonyl)-1-piperazinyl]phenyl}amino)-methy]imidazol-1-ylmethyl}benzonitrile,
and also the therapeutically acceptable salts and solvates thereof.

12. A process for preparing the compounds of general formula (I) as claimed in one of claims 1 to 11, characterized in that an intermediate of general formula (V) 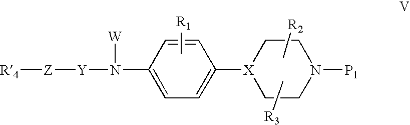
in which R₁, R₂, R₃, W, X, Y and Z are defined as above, R′₄ represents a precursor of R₄ and P₁ represents a protecting group which is removed just before the condensation to give the free amine, is condensed with an intermediate of formula R₅-L₃ in which R₅ is defined as above and L₃ represents either a leaving group such as, for example, Cl, Br, I, OSO₂CH₃, OSO₂CF₃ or O-tosyl, or a fluorine atom, or the species R₅-L₃ may also represent an isocyanate or an isothiocyanate.

13. The process for preparing the compounds of general formula (I) as claimed in one of claims 1 to 11, characterized in that an intermediate of general formula (VIII) 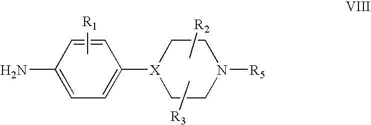
in which R₁, R₂, R₃, X and R₅ are defined as above, is condensed with an intermediate of general formula R′₄—Z—Y—L₁ in which Z and Y are defined as above, R′₄ represents a precursor of R₄ and L₁ represents either a leaving group such as, for example, Cl, Br, I, OSO₂CH₃, OSO₂CF₃ or O-tosyl, or a hydroxyl; in this case, the reaction with the amine of general formula (VIII) amounts to the formation of an amide by condensation between this amine and a carboxylic acid derivative.

14. A pharmaceutical composition containing, as active substance, a compound as claimed in one of claims 1 to 11 in combination with an acceptable pharmaceutical vehicle.

15. A pharmaceutical composition containing, as active substance, a compound as claimed in one of claims 1 to 11 in combination with an acceptable pharmaceutical vehicle, for the curative or preventive treatment of disorders associated with farnesylation and/or geranylgeranylation of proteins.

16. A pharmaceutical composition containing, as active substance, a compound as claimed in claims 1 to 11 in combination with an acceptable pharmaceutical vehicle, for treating or preventing cancers such as cancer of the lung, of the pancreas, of the skin, of the head, of the neck, of the uterus, of the ovaries, anal cancer, cancer of the stomach, of the colon, of the breast, of the esophagus, of the small intestine, of the thyroid gland, of the prostate, of the kidney, of the bladder, acute or chronic leukemia, or a combination of two or more of these cancers.

17. A pharmaceutical composition as claimed in one of claims 14 to 16 in combination especially with an anticancer agent such as cytotoxic anticancer agents, especially navelbine, vinflunine, taxol, taxotere, 5-fluorouracil, le methotrexate, doxorubicin, camptothecin, gemcitabine, etoposide, cisplatin or BCNU, or hormonal anticancer agents, for instance tamoxifen or medroxyprogesterone, for their simultaneous, separate or sequential use, for the treatment or prevention of cancers.

18. The pharmaceutical composition as claimed in one of claims 14 to 16, in combination with an agent for inhibiting the biosynthesis of farnesyl and geranylgeranyl pyrophosphates, such as an HMG-CoA reductase inhibitor, for instance lovastatin, simvastatin, pravastatin, fluvastatin, atorvastatin or cerivastatin, for their simultaneous, separate or sequential use.

19. The pharmaceutical composition as claimed in one of claims 14 to 16, in combination with a treatment with radiation (X-rays or gamma rays) for their simultaneous, separate or sequential use for treating or preventing cancers.

20. The pharmaceutical composition as claimed in one of claims 14 to 16, for treating or preventing restenosis or atherosclerosis.

21. The pharmaceutical composition as claimed in one of claims 14 to 16, for treating or preventing infections associated with PFTase such as delta hepatitis.

22. The pharmaceutical composition as claimed in one of claims 14 to 16, for treating or preventing benign proliferative disorders.

23. A compound of formula (I): 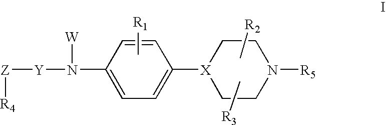
in which
W represents
hydrogen, COR₆, CSR₆, SO₂R₆, CO(CH₂)ₙR₆, (CH₂)ₙR₇,
X represents:
CH or N,
Y represents:
(CH₂)ₙ, CO, CH₂CO, CH═CHCO, CH₂CH₂CO,
when Y=CO, CH₂CO, CH═CH—CO or CH₂CH₂—CO then W represents only hydrogen,
Z represents:
imidazole, benzimidazole, isoxazole, tetrazole, oxadiazole, thiazole, thiadiazole, pyridine, quinazoline, quinoxaline, these heterocycles may be unsubstituted or substituted with one or more groups chosen from C₁-C₁₅ alkyl, halogen, OMe, CN, NO₂, OH, CF₃, OCF₃, OCH₂Ph, SMe, COOMe, COOEt, COOH, CONHOH, SO₂NH₂, CONH₂,
When Z=pyridine, then Y is other than CO,
R₁ represents:
hydrogen, C₁-C₆ alkyl, halogen, OCH₃, CF₃,
R₂ and R₃, which may be identical or different, represent:
hydrogen, C₁-C₆ alkyl,
R₄ represents:
a) hydrogen,
b) C₁-C₆ alkyl which is unsubstituted or substituted with an aryl, a cyanophenyl, a nitrophenyl, an aminophenyl, a methoxyphenyl, a hydroxyphenyl, a heterocycle, a halogen, CN, NO₂, OR₂, SR₂, NR₂R₃ COOR₂,
c) an aryl,
d) a heterocycle,
R₅ represents:
hydrogen, COR₇, SO₂R₇, CO(CH₂)ₙSR₇, CO(CH₂)ₙOR₇, CONR₇R₈, CSNR₇R₈, CO(CH₂)ₘCOR₇,
R₆ represents:
a) a phenyl or a naphthyl which is unsubstituted or substituted with one or more residues chosen from C₁-C₆ alkyl, halogen, phenyl, naphthyl, NO₂, CN, CF₃, OR₇, SR₇, NR₇R, COOR₇, CONR₇R₈, COR₇,
b) a C₁-C₆ alkyl, a cycloalkyl,
c) a heterocycle,
d) NR₇R₈,
R₇ and R₈, which may be identical or different, represent:
a) hydrogen; C₁-C₁₅ alkyl, which is unsubstituted or substituted with a halogen, COOMe, COOH, OMe, OH, CF₃, CN, SMe; cycloalkyl which is unsubstituted or substituted with a halogen, OMe, OH, CF₃, CN, SMe; alkylcycloalkyl which is unsubstituted or substituted with a halogen, OMe, OH, CF₃, CN, SMe; or R₇ and R₈ when they are adjacent, taken together, can form a 4- to 6-membered ring with the nitrogen atom to which they are attached,
b) a heterocycle, an alkylheterocycle,
c) an aryl, an alkylaryl,
n represents:
0 to 10,
m represents:
2 to 10,
with the proviso that when Z represents a quinazoline or benzimidazole group, then R₅ is other than CH₂Ph or methyl and n is other than zero,
and also the therapeutically acceptable salts and solvates thereof, the abovementioned compound possibly being in the form of pure optical isomers or mixtures of optical isomers in all proportions, including the racemic form.

24. A compound of claim 23, wherein R₁, R₂, R₃ and R₄ each represent a hydrogen and Y is a methylene (CH₂).

25. A compound of claim 23, wherein Z represents an imidazolyl or pyridyl residue.

26. A compound of claim 25, wherein Z represents an imidazolyl residue and R₄ is a benzyl group substituted with a nitrile, nitro or methoxy group in position 4.

27. A compound of claim 23, wherein X represents a nitrogen atom.

28. A compound of claim 23, wherein X represents a carbon atom.

29. A compound of claim 23, wherein R₅ represents 2-thiophenecarbonyl or 5-chloro-2-thiophenecarbonyl.

30. A compound of claim 23, wherein R₅ represents CSNR₇R₈

31. A compound of claim 23, wherein W represents COR₆.

32. A compound of claim 23, wherein W represents (CH₂)ₙR₇.

33. A compound of claim 23, selected from:
N-(3H-imidazol-4-ylmethyl)-4-nitro-N-{4-[4-(thiophene-2-carbonyl)-1-piperazinyl]phenyl}benzamide,
4-{4-[benzenesulfonyl-(3H-imidazol-4-ylmethyl)amino]phenyl}-1-piperazinecarbothioic acid propylamide,
4-{4-[benzenesulfonyl-(3H-imidazol-4-ylmethyl)amino]phenyl}-1-piperazinecarbothioic acid cyclohexylamide,
4-{4-[benzenesulfonyl-(3H-imidazol-4-ylmethyl)amino]phenyl}-1-piperazinecarbothioic acid cyclohexylmethyl-amide,
4-{4-[benzenesulfonyl-(3H-imidazol-4-ylmethyl)amino]phenyl}-1-piperazinecarbothioic acid benzylamide,
4-{4-[benzenesulfonyl-(3H-imidazol-4-ylmethyl)amino]phenyl}-1-piperazinecarbothioic acid isobutylamide,
4-{4-[benzenesulfonyl-(3H-imidazol-4-ylmethyl)amino]phenyl}-1-piperazinecarbothioic acid (2-methoxyethyl)amide,
N-{4-[4-(5-chloro-2-thiophenecarbonyl)-1-piperazinyl]phenyl}-3-fluoro-N-(3H-imidazol-4-ylmethyl)benzamide,
4-[5-({4-[4-(5-chloro-2-thiophenecarbonyl)-1-piperazinyl]phenylamino}methyl)-imidazol-1-ylmethyl]benzonitrile,
N-{4-[4-(5-chloro-2-thiophenecarbonyl)-1-piperazinyl]phenyl}-N-[3-(4-cyanobenzyl)-3H-imidazol-4-ylmethyl]benzamide,
N-{4-[4-(5-chloro-2-thiophenecarbonyl)-1-piperazinyl]phenyl}-N-[3-(4-cyanobenzyl)-3H-imidazol-4-ylmethyl]benzenesulfonamide,
N-{4-[4-(5-chloro-2-thiophenecarbonyl)-1-piperazinyl]phenyl}-4-cyano-N-(3H-imidazol-4-ylmethyl)benzamide,
thiophene-2-carboxylic acid {4-[4-(5-chloro-2-thiophenecarbonyl)-1-piperazinyl]phenyl}[3-(4-cyanobenzyl)-3H-imidazol-4-ylmethyl]amide,
2-chloro-N-{4-[4-(5-chloro-2-thiophenecarbonyl)-1-piperazinyl]phenyl}-N-[3-(4-cyanobenzyl)-3H-imidazol-4-ylmethyl]benzamide,
3-chloro-N-{4-[4-(5-chloro-2-thiophenecarbonyl)-1-piperazinyl]phenyl}-N-[3-(4-cyanobenzyl)-3H-imidazol-4-ylmethyl]benzamide,
4-chloro-N-{4-[4-(5-chloro-2-thiophenecarbonyl)-1-piperazinyl]phenyl}-N-[3-(4-cyanobenzyl)-3H-imidazol-4-ylmethyl]benzamide,
N-{4-[4-(5-chloro-2-thiophenecarbonyl)-1-piperazinyl]phenyl}-N-[3-(4-cyanobenzyl)-3H-imidazol-4-ylmethyl]-2-fluorobenzamide,
N-{4-[4-(5-chloro-2-thiophenecarbonyl)-1-piperazinyl]phenyl}-N-[3-(4-cyanobenzyl)-3H-imidazol-4-ylmethyl]-3-fluorobenzamide,
N-{4-[4-(5-chloro-2-thiophenecarbonyl)-1-piperazinyl]phenyl}-N-[3-(4-cyanobenzyl)-3H-imidazol-4-ylmethyl]-4-fluorobenzamide,
N-{4-[4-(5-chloro-2-thiophenecarbonyl)-1-piperazinyl]phenyl}-N-[3-(4-cyanobenzyl)-3H-imidazol-4-ylmethyl]-2-trifluoromethylbenzamide,
N-{4-[4-(5-chloro-2-thiophenecarbonyl)-1-piperazinyl]phenyl}-N-[3-(4-cyanobenzyl)-3H-imidazol-4-ylmethyl]-2-methoxybenzamide,
N-{4-[4-(5-chloro-2-thiophenecarbonyl)-1-piperazinyl]phenyl}-N-[3-(4-cyanobenzyl)-3H-imidazol-4-ylmethyl]-3-methoxybenzamide,
N-{4-[4-(5-chloro-2-thiophenecarbonyl)-1-piperazinyl]phenyl}-N-[3-(4-cyanobenzyl)-3H-imidazol-4-ylmethyl]-4-methoxybenzamide,
N-{4-[45-chloro-2-thiophenecarbonyl)-1-piperazinyl]phenyl}-N-[3-(4-cyanobenzyl)-3H-imidazol-4-ylmethyl]-2-nitrobenzamide,
N-{4-[4-(5-chloro-2-thiophenecarbonyl)-1-piperazinyl]phenyl}-N-[3-(4-cyanobenzyl)-3H-imidazol-4-ylmethyl]-3-nitrobenzamide,
N-{4-[4-(5-chloro-2-thiophenecarbonyl)-1-piperazinyl]phenyl}-N-[3-(4-cyanobenzyl)-3H-imidazol-4-ylmethyl]-4-nitrobenzamide,
N-{4-[4-(5-chloro-2-thiophenecarbonyl)-1-piperazinyl]phenyl}-4-cyano-N-[3-(4-cyanobenzyl)-3H-imidazol-4-ylmethyl]-benzamide,
cyclohexanecarboxylic acid {4-[4-(5-chloro-thiophene-2-carbonyl)-1-piperazinyl]phenyl}-[3-(4-cyanobenzyl)-3H-imidazol-4-ylmethyl]-amide,
N-{4-[4-(5-chloro-2-thiophenecarbonyl)-1-piperazinyl]phenyl}-2-cyano-N-[3-(4-cyanobenzyl)-3H-imidazol-4-ylmethyl]-benzenesulfonamide,
N-{4-[4-(5-chloro-2-thiophenecarbonyl)-1-piperazinyl]phenyl}-3-cyano-N-[3-(4-cyanobenzyl)-3H-imidazol-4-ylmethyl]benzenesulfonamide,
N-{4-[4-(5-chloro-2-thiophenecarbonyl)-1-piperazinyl]phenyl}-4-cyano-N-[3-(4-cyanobenzyl)-3H-imidazol-4-ylmethyl]benzenesulfonamide,
1-{4-[4-(5-chloro-2-thiophenecarbonyl)-1-piperazinyl]phenyl}-1-(3H-imidazol-4-ylmethyl)-3propylurea,
1-{4-[4-(5-chloro-2-thiophenecarbonyl)-1-piperazinyl]phenyl}-1-(3H-imidazol-4-ylmethyl)-3-(2-methylsulfanylphenyl)urea,
N-(3H-imidazol-4-ylmethyl)-4-nitro-N-[4-(4-propylthiocarbamoyl-1-piperazinyl)phenyl]benzamide,
N-[4-(4-cyclohexylthiocarbamoyl-1-piperazinyl)phenyl]-N-(3H-imidazol-4-ylmethyl)-4-nitrobenzamide,
N-(3H-imidazol-4-ylmethyl)-N-[4-(4-isobutylthiocarbamoyl-1-piperazinyl)phenyl]-4-nitrobenzamide,
4-{4-[benzenesulfonyl-(3H-imidazol-4-ylmethyl)amino]phenyl}piperidine-1-carbothioic acid isobutylamide,
N-(3H-imidazol-4-ylmethyl)-N-[4-(1-isobutylthiocarbamoylpiperidin-4-yl)phenyl]-4-nitrobenzamide,
cyclohexanecarboxylic acid (3H-imidazol-4-ylmethyl)-[4-(1-isobutylthiocarbamoyl-piperidin-4-yl)-phenyl]amide,
4-{4-[(4-cyano-benzenesulfonyl)(3H-imidazol-4-ylmethyl)amino]phenyl}piperidine-1-carbothioic acid cyclohexylamide,
N-{4-[4-(5-chloro-2-thiophenecarbonyl)-1-piperazinyl]phenyl}-3-(3H-imidazol-4-yl)acrylamide,
N-{4-[4-(5-chloro-2-thiophenecarbonyl)-1-piperazinyl]phenyl}-3-[3-(4-cyano-benzyl)-3H-imidazol-4-yl]propionamide,
4-(5-{[{4-[4-(5-chloro-2-thiophenecarbonyl)-1-piperazinyl]phenyl}(3-fluoro-benzyl)amino]methyl}imidazol-1-ylmethyl)benzonitrile,
4-(5-{[{4-[4-(5-chloro-2-thiophenecarbonyl)-1-piperazinyl]phenyl}(4-fluoro-benzyl)amino]methyl}imidazol-1-ylmethyl)benzonitrile,
4-(5-{[{4-[4-(5-chloro-2-thiophenecarbonyl)-1-piperazinyl]phenyl}(3-hydroxy-benzyl)amino]methyl}imidazol-1-ylmethyl)-benzonitrile,
4-(5-{[{4-[4-(5-chloro-2-thiophenecarbonyl)-1-piperazinyl]phenyl}(3-methoxy-benzyl)amino]methyl}imidazol-1-ylmethyl)benzonitrile,
4-(5-{[{4-[4-(5-chloro-2-thiophenecarbonyl)-1-piperazinyl]phenyl}-(3,4-dichloro-benzyl)amino methyl}imidazol-1-ylmethyl)benzonitrile,
4-{5-[({4-[4-(5-chloro-2-thiophenecarbonyl)-1-piperazinyl]phenyl}thiophen-3-ylmethylamino)methyl]imidazol-1-ylmethyl}benzonitrile,
4-{5-[(benzo[1,3]dioxol-5-ylmethyl-{4-[4-(5-chloro-2-thiophenecarbonyl)-1-piperazinyl]phenyl}amino)methyl]imidazol-1-ylmethyl}benzonitrile,
4-{5-[(butyl-{4-[4-(5-chloro-2-thiophenecarbonyl)-1-piperazinyl]phenyl}amino)-methyl]imidazol-1-ylmethyl}benzonitrile,
4-(5-{[{4-[4-(5-chloro-2-thiophenecarbonyl)-1-piperazinyl]phenyl}(3-phenyl-propyl)amino]methyl}imidazol-1-ylmethyl)benzonitrile,
4-{5-[((3-fluorobenzyl)-{4-[4-(thiophene-2-carbonyl)-1-piperazinyl]phenyl}amino)-methyl]imidazol-1-ylmethyl}benzonitrile,
4-{5-[((4-fluorobenzyl)-{4-[4-(thiophene-2-carbonyl)-1-piperazinyl]phenyl}amino)-methyl]imidazol-1-ylmethyl}benzonitrile,
4-{5-[((4-fluorobenzyl)-{4-[4-(thiophene-2-carbonyl)-1-piperazinyl]phenyl}amino)-methyl]imidazol-1-ylmethyl}benzonitrile,
4-{5-[((3-methoxybenzyl)-{4-[4-(thiophene-2-carbonyl)-1-piperazinyl]phenyl}-amino)-methyl]imidazol-1-ylmethyl}benzonitrile,
4-{5-[((3,4-dichlorobenzyl)-{4-[4-(thiophene-2-carbonyl)-1-piperazinyl]phenyl}-amino)methyl]imidazol-1-ylmethyl}benzonitrile,
4-{5-[({4-[4-(thiophene-2-carbonyl)-1-piperazinyl]phenyl}thiophen-3-ylmethyl-amino)methyl]imidazol-1-ylmethyl}benzonitrile,
4-{5-[(benzo[1,3]dioxol-5-ylmethyl-{4-[4-(thiophene-2-carbonyl)-1-piperazinyl]phenyl}amino)methyl]imidazol-1-ylmethyl}benzonitrile,
4-{5-[(butyl-{4-[4-(thiophene-2-carbonyl)-1-piperazinyl]phenyl}amino)methyl]-imidazol-1-ylmethyl}benzonitrile,
4-{5-[((3-phenylpropyl)-{4-[4-(thiophene-2-carbonyl)-1-piperazinyl]phenyl}-amino)methyl]imidazol-1-ylmethyl}benzonitrile,
N-{4-[4-(5-chloro-2-thiophenecarbonyl)-1-piperazinyl]phenyl}-N-(1-methyl-1H-benzoimidazol-2-ylmethyl)benzamide,
N-{4-[4-(5-chloro-2-thiophenecarbonyl)-1-piperazinyl]phenyl}-N-(2-methyl-3H-imidazol-4-ylmethyl)benzamide,
N-{4-[4-(5-chloro-2-thiophenecarbonyl)-1-piperazinyl]phenyl}-N-thiazol-2-ylmethylbenzamide,
N-{4-[4-(3-chloro-2-thiophenecarbonyl)-1-piperazinyl]phenyl}-N-[3-(4-cyanobenzyl)-3H-imidazol-4-ylmethyl]benzamide,
N-{4-[4-(benzo[b]thiophene-2-carbonyl)-1-piperazinyl]phenyl}-N-[3-(4-cyanobenzyl)-3H-imidazol-4-ylmethyl]benzamide,
N-{4-[4-(3-chlorobenzo[b]thiophene-2-carbonyl)-1-piperazinyl]phenyl}-N-[3-(4-cyanobenzyl)-3H-imidazol-4-ylmethyl]benzamide,
N-[3-(4-cyanobenzyl)-3H-imidazol-4-ylmethyl]-N-{4-[4-(2-thiophen-2-ylacetyl)-1-piperazinyl]phenyl}benzamide,
N-[3-(4-cyanobenzyl)-3H-imidazol-4-ylmethyl]-N-{4-[4-(furan-2-carbonyl)-1piperazinyl]phenyl}benzamide,
N-[3-(4-cyanobenzyl)-3H-imidazol-4-ylmethyl]-N-{4-[4-(isoxazole-5-carbonyl)-1-piperazinyl]phenyl}benzamide,
N-(4-{4-[3-(2-chlorophenyl)-5-methylisoxazole-4-carbonyl]-1-piperazinyl}phenyl)-N-[3-(4-cyanobenzyl)-3H-imidazol-4-ylmethyl]benzamide,
N-[3-(4-cyanobenzyl)-3H-imidazol-4-ylmethyl]-N-{4-[4-(5-methylthiophene-2-carbonyl)-1-piperazinyl]phenyl}benzamide,
N-{4-[4-(5-bromothiophene-2-carbonyl)-1-piperazinyl]phenyl}-N-[3-(4-cyanobenzyl)-3H-imidazol-4-ylmethyl]benzamide,
N-[3-(4-cyanobenzyl)-3H-imidazol-4-ylmethyl]-N-{4-[4-(thiophene-3-carbonyl)-1-piperazinyl]phenyl}benzamide,
N-[3-(4-cyanobenzyl)-3H-imidazol-4-ylmethyl]-N-{4-[4-(2-thiophen-3-ylacetyl)-1-piperazinyl]phenyl}benzamide,
N-[3-(4-cyanobenzyl)-3H-imidazol-4-ylmethyl]-N-{4-[4-(4-thiophen-2-ylbutyryl)-1-piperazinyl]phenyl}benzamide,
N-(4-{4-[2-(5-chlorobenzo[b]thiophen-3-yl)acetyl]-1-piperazinyl}phenyl)-N-[3-(4-cyanobenzyl)-3H-imidazol-4-ylmethyl]benzamide,
N-[3-(4-cyanobenzyl)-3H-imidazol-4-ylmethyl]-N-{4-[4-(4-oxo-4-thiophen-2-ylbutyryl)-1-piperazinyl]phenyl}benzamide,
N-[3-(4-cyanobenzyl)-3H-imidazol-4-ylmethyl]-N-{4-[4-(5-oxo-5-thiophen-2-ylpentanoyl)-1-piperazinyl]phenyl}benzamide,
N-[3-(4-cyanobenzyl)-3H-imidazol-4-ylmethyl]-N-{4-[4-(1-methyl-1H-pyrrole-2-carbonyl)-1-piperazinyl]phenyl}benzamide,
N-(4-{4-[5-(4-chlorophenyl)-furan-2-carbonyl]-1-piperazinyl}-phenyl)-N-[3-(4-cyanobenzyl)-3H-imidazol-4-ylmethyl]benzamide,
N-[3-(4-cyanobenzyl)-3H-imidazol-4-ylmethyl]-N-{4-[4-(5-methyl-1-phenyl-1H-pyazole-4-carbonyl)-1-piperazinyl]phenyl}benzamide,
N-[3-(4-cyanobenzyl)-3H-imidazol-4-ylmethyl]-N-{4-[4-(3,5-dimethylisoxazole-4-carbonyl)-1-piperazinyl]phenyl}benzamide,
N-[3-(4-cyanobenzyl)-3H-imidazol-4-ylmethyl]-N-{4-[4-(5-methyl-2-phenyl-2H-[1,2,3]triazole-4-carbonyl)-1-piperazinyl]phenyl}benzamide,
N-{4-[4-(5-chlorothiophen-2-ylmethyl)-1-piperazinyl]phenyl}-N-[3-(4-cyanobenzyl)-3H-imidazol-4-ylmethyl]benzamide,
N-[3-(4-cyanobenzyl)-3H-imidazol-4-ylmethyl]-N-{4-[4-(3-methylthiophen-2-ylmethyl)-1-piperazinyl]phenyl}benzamide,
N-[3-(4-cyanobenzyl)-3H-imidazol-4-ylmethyl]-N-{4-[4-(5-methylthiophen-2-ylmethyl)-1-piperazinyl]phenyl}benzamide,
N-[3-(4-cyanobenzyl)-3H-imidazol-4-ylmethyl]-N-{4-[4-(5-ethylthiophen-2-ylmethyl)-1-piperazinyl]phenyl}benzamide,
N-{4-[4-(5-chloro-2-thiophenecarbonyl)-1-piperazinyl]phenyl}-N-(2-phenyl-1-Himidazol-4-ylmethyl)benzamide,
4-(4-{benzenesulfonyl-[3-(4-cyanobenzyl)-3H-imidazol-4-ylmethyl]amino}phenyl)-1-piperazinecarbothioic acid isobutylamide,
4-(4-{benzenesulfonyl-[3-(4-cyanobenzyl)-3H-imidazol-4-ylmethyl]amino}phenyl)-1-piperazinecarbothioic acid propylamide,
4-(4-{benzenesulfonyl-[3-(4-cyanobenzyl)-3H-imidazol-4-ylmethyl]amino}phenyl)-1-piperazinecarbothioic acid cyclohexylamide,
4-(4-{benzenesulfonyl-[3-(4-cyanobenzyl)-3H-imidazol-4-ylmethyl]amino}phenyl)-1-piperazinecarbothioic acid benzylamide,
4-(4-{(4-cyanobenzenesulfonyl)-[3-(4-cyanobenzyl)-3H-imidazol-4-ylmethyl]amino}phenyl)-1-piperazinecarbothioic acid isobutylamide,
4-(4-{(4-cyanobenzenesulfonyl)-[3-(4-cyanobenzyl)-3H-imidazol-4-ylmethyl]amino}phenyl)-1-piperazinecarbothioic acid propylamide,
4-(4-{(4-cyanobenzenesulfonyl)-[3-(4-cyanobenzyl)-3H-imidazol-4-ylmethyl]amino}phenyl)-1-piperazinecarbothioic acid cyclohexylamide,
4-(4-{(4-cyanobenzenesulfonyl)-[3-(4-cyanobenzyl)-3H-imidazol-4-ylmethyl]amino}phenyl)-1-piperazinecarbothioic acid cyclohexylmethylamide,
4-(4-{(4-cyanobenzenesulfonyl)-[3-(4-cyanobenzyl)-3H-imidazol-4-ylmethyl]amino}phenyl)-1-piperazinecarbothioic acid benzylamide,
4-cyano-N-[3-(4-cyanobenzyl)-3H-imidazol-4-ylmethyl]-N-[4-(4-isobutylthiocarbamoyl-1-piperazinyl)phenyl]benzamide,
4-cyano-N-[3-(4-cyanobenzyl)-3H-imidazol-4-ylmethyl]-N-[4-(4-propylthiocarbamoyl-1-piperazinyl)phenyl]benzamide,
4-cyano-N-[3-(4-cyanobenzyl)-3H-imidazol-4-ylmethyl]-N-[4-(4-cyclohexylthiocarbamoyl-1-piperazinyl)phenyl]benzamide,
4-cyano-N-[3-(4-cyanobenzyl)-3H-imidazol-4-ylmethyl]-N-{4-[4-(cyclohexylmethylthiocarbamoyl)-1-piperazinyl]phenyl}benzamide,
N-[4-(4-benzylthiocarbamoyl-1-piperazinyl)-phenyl]-4-cyano-N-[3-(4-cyanobenzyl)-3H-imidazol-4-ylmethyl]benzamide,
N-[3-(4-cyano-benzyl)-3H-imidazol-4-ylmethyl]-3-fluoro-N-[4-(4-isobutylthiocarbamoyl-1-piperazinyl)-phenyl]benzamide,
N-[3-(4-cyanobenzyl)-3H-imidazol-4-ylmethyl]-3-fluoro-N-[4-(4-propylthiocarbamoyl-1-piperazinyl)phenyl]benzamide,
N-[3-(4-cyanobenzyl)-3H-imidazol-4-ylmethyl]-N-[4-(4-cyclohexylthiocarbamoyl1-piperazinyl)phenyl]-3-fluorobenzamide,
N-[4-(4-benzylthiocarbamoyl-1-piperazinyl)phenyl]-N-[3-(4-cyanobenzyl)-3-Himidazol-4-ylmethyl]-3-fluorobenzamide,
4-{4-[1-[3-(4-cyanobenzyl)-3H-imidazol-4-ylmethyl]-3-(2-methylsulfanylpheenyl)-ureido]phenyl}-1-piperazinecarbothioic acid propylamide,
4-(4-{1-[3-(4-cyanobenzyl)-3H-imidazol-4-ylmethyl]-3-propylureido}phenyl)-1-piperazinecarbothioic acid isobutylamide,
4-(4-{1-[3-(4-cyanobenzyl)-3H-imidazol-4-ylmethyl]-3-propylureido}phenyl)-1-piperzinecarbothioic acid propylamide,
4-(4-{1-[3-(4-cyanobenzyl)-3H-imidazol-4-ylmethyl]-3-propylureido}phenyl)-1-piperzinecarbothioic acid cyclohexylamide,
4-(4-{1-[3-(4-cyanobenzyl)-3H-imidazol-4-ylmethyl]-3-propylureido}phenyl)-1-piperzinecarbothioic acid cyclohexylmethylamide,
4-(4-{1-[3-(4-cyanobenzyl)-3H-imidazol-4-ylmethyl]-3-propylureido}phenyl)-1-piperazinecarbothioic acid benzylamide,
N-(3H-imidazol-4-ylmethyl)-N-[4-(4-isobutylthiocarbamoyl-1-piperazinyl)phenyl]-3-methoxybenzamide,
3-fluoro-N-(3H-imidazol-4-ylmethyl)-N-[4-(4-isobutylthiocarbamoyl-1-piperazinyl)phenyl]benzamide,
cyclopropanecarboxylic acid {4-[4-(5-chloro-2-thiophenecarbonyl)-1-piperazinyl]phenyl}-]3-(4-cyanobenzyl)-3H-imidazol-4-ylmethyl]amide,
cyclobutanecarboxylic acid {4-[4-(5-chloro-2-thiophenecarbonyl)-1-piperazinyl]phenyl}-3-(4-cyanobenzyl)-3H-imidazol-4-ylmethyl]amide,
cyclopentanecarboxylic acid {4-[4-(5-chloro-2-thiophenecarbonyl)-1-piperazinyl]phenyl}-[3-(4-cyanobezyl)-3H-imidazol-4-ylmethyl]amide,
N-{4-[4-(5-chloro-2-thiophenecarbonyl)-1-piperazinyl]phenyl}-N-[3-(4-cyanobenzyl)-3H-imidazol-4-ylmethyl]-3-cyclopentylpropionamide,
N-{4-[4-(5-chloro-2-thiophenecarbonyl)-1-piperazinyl]phenyl}-N-[3-(4-cyanobenzyl)-3H-imidazol-4-ylmethyl]-2-cyclopentylacetamide,
naphthalene-1-carboxylic acid {4-[4-(5-chloro-2-thiophenecarbonyl)-1-piperazinyl]phenyl}-[3-(4-cyanobenzyl)-3H-imidazol-4-ylmethyl]amide,
biphenyl-4-carboxylic acid {4-[4-(5-chloro-2-thiophenecarbonyl)-1-piperazinyl]phenyl}-[3-(4-cyanobenzyl)-3H-imidazol-4-ylmethyl]amide,
N-{4-[4-(5-chloro-2-thiophenecarbonyl)-1-piperazinyl]phenyl}-N-[3-(4-cyanobenzyl)-3-H-imidazol-4-ylmethyl]-3,5-dimethoxybenzamide,
N-{4-[4-(5-chloro-2-thiophenecarbonyl)-1-piperazinyl]phenyl}-N-[3-(4-cyanobenzyl)-3H-imidazol-4-ylmethyl]-3-methylbenzamide,
N-{4-[4-(5-chloro-2-thiophenecarbonyl)-1-piperazinyl]phenyl}-N-[3-(4-cyanobenzyl)-3H-imidazol-4-ylmethyl]-4-etoxybenzamide,
N-{4-[4-(5-chloro-2-thiophenecarbonyl)-1-piperazinyl]phenyl}-N-[3-(4-cyanobenzyl)-3-H-imidazol-4-ylmethyl]-4-ethylbenzamide,
N-{4-[4-(5-chloro-2-thiophenecarbonyl)-1-piperazinyl]phenyl}-N-[3-(4-cyanobenzyl)-3H-imidazol-4-ylmethyl]-9-hexylbenzamide,
N-{4-[4-(5-chloro-2-thiophenecarbonyl)-1-piperazinyl]phenyl}-N-[3-(4-cyanobenzyl)-3H-imidazol-4-ylmethyl]-4-methylbenzamide,
4-butyl-N-{4-[4-(5-chloro-2-thiophenecarbonyl)-1-piperazinyl]phenyl}-N-[3-(4-cyanobenzyl)-3H-imidazol-4-ylmethyl]benzamide,
N-{4-[4-(5-chloro-2-thiophenecarbonyl)-1-piperazinyl]phenyl}-N-[3-(4-cyanobenzyl)-3H-imidazol-4-ylmethyl]-3,4-difluorobenzamide,
N-{4-[4-(5-chloro-2-thiophenecarbonyl)-1-piperazinyl]phenyl}-N-[3-(4-cyanobenzyl)-3H-imidazol-4-ylmethyl]-3-trifluoromethoxybenzamide,
N-{4-[4-(5-chloro-2-thiophenecarbonyl)-1-piperazinyl]phenyl}-N-[3-(4-cyanobenzyl)-3H-imidazol-4-ylmethyl]acetamide,
N-{4-[4-(5-chloro-2-thiophenecarbonyl)-1-piperazinyl]phenyl}-N-[3-(4-cyanobenzyl)-3-imidazol-4-ylmethyl]-2-phenoxyacetamide,
N-{4-[4-(5-chloro-2-thiophenecarbonyl)-1-piperazinyl]phenyl}-N-[3-(4-cyanobenzyl)-3H-imidazol-4-ylmethyl]-2-phenylacetamide,
N-{4-[4-(5-chloro-2-thiophenecarbonyl)-1-piperazinyl]phenyl}-N-[3-(4-cyanobenzyl)-3H-imidazol-4-ylmethyl]propionamide,
N-{4-[4-(5-chloro-2-thiophenecarbonyl)-1-piperazinyl]phenyl}-N-[3-(4-cyanobenzyl)-3H-imidazol-4-ylmethyl]butyramide,
furan-2-carboxylic acid {4-[4-(5-chloro-2-thiophenecarbonyl)-1-piperazinyl]phenyl}-[3-(4-cyanobenzyl)-3H-imidazol-4-ylmethyl]amide,
N-{4-[4-(5-chloro-2-thiophenecarbonyl)-1-piperazinyl]phenyl}-N-[3-(4-cyanobenzyl)-3H-imidazol-4-ylmethyl]-2-thiophen-2-ylacetamide,
N-{4-[4-(5-chloro-2-thiophenecarbonyl)-1-piperazinyl]phenyl}-N-[3-(4-cyanobenzyl)-3H-imidazol-4-ylmethyl]-methanesulfonamide,
N-{4-[4-(5-chloro-2-thiophenecarbonyl)-1-piperazinyl]phenyl}-N-[3-(4-cyanobenzyl)-3H-imidazol-4-ylmethyl]-3,4-dimethylbenzarnide,
N-{4-[4-(5-chloro-2-thiophenecarbonyl)-1-piperazinyl]phenyl}-N-[3-(4-cyanobenzyl)-3H-imidazol-4-ylmethyl]-3-fluoro-4-methylbenzamide,
2-chloro-N-{4-[4-(5-chloro-2-thiophenecarbonyl)-1-piperazinyl]phenyl}-N-[3-(4-cyanobenzyl)-3H-imidazol-4-ylmethyl]-5-methylsulfanylbenzamide,
3-methoxycyclohexanecarboxylic acid {4-[4-(5-chloro-2-thiophenecarbonyl)-1-piperazinyl]phenyl}[3-(4-cyanobenzyl)-3H-imidazol-4-ylmethyl]amide,
4-methylcyclohexanecarboxylic acid {4-[4-(5-chloro-2-thiophenecarbonyl)-1-piperazinyl]phenyl}[3-(4-cyanobenzyl)-3H-imidazol-4-ylmethyl]amide,
1-methylcyclohexanecarboxylic acid {4-[4-(5-chloro-2-thiophenecarbonyl)-1-piperazinyl]phenyl}-[3-(4-cyanobenzyl)-3H-imidazol-4-ylmethyl]amide,
N-{4-[4-(5-chloro-2-thiophenecarbonyl)-1-piperazinyl]phenyl}-N-[3-(4-cyanobenzyl)-3H-imidazol-4-ylmethyl]-2-naphthalen-1-ylacetamide,
2-phenylcyclopropanecarboxylic acid {4-[4-(5-chloro-2-thiophenecarbonyl)-1-piperazinyl]phenyl}[3-(4-cyanobenzyl)-3H-imidazol-4-ylmethyl]amide,
4-{5-[(benzyl-{4-[4-(5-chloro-2-thiophenecarbonyl)-1-piperazinyl]phenyl}amino)-methyl]imidazol-1-ylmethyl}benzonitrile,
and the therapeutically acceptable salts and solvates thereof.

34. A process for preparing a compound of claim 23, wherein an intermediate of general formula (V) 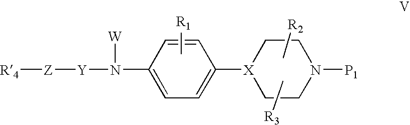
in which R′₄ is a precursor of R₄ and P₁ is a protecting group which is removed just before the condensation to give the free amine, is condensed with an intermediate of formula R₅-L₃ in which L₃ is either a leaving group such as Cl, Br, I, OSO₂CH₃, OSO₂CF₃ or O-tosyl, or a fluorine atom, or the species R₅-L₃ is an isocyanate or an isothiocyanate.

35. The process for preparing a compound of claim 23, wherein an intermediate of general formula (VIII) 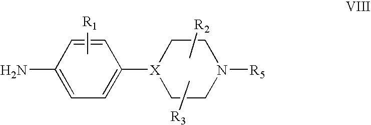
is condensed with an intermediate of general formula R′₄—Z—Y—L₁ in which R′₄ is a precursor of R₄ and L₁ is either a leaving group such as Cl, Br, I, OSO₂CH₃, OSO₂CF₃ or O-tosyl, or a hydroxyl; wherein the reaction with the amine of general formula (VIII) amounts to the formation of an amide by condensation between this amine and a carboxylic acid derivative.

36. A pharmaceutical composition containing, as active principal, a compound of claim 23 in combination with a pharmaceutically acceptable vehicle.

37. A method for treating a living animal body afflicted with a condition selected from disorders associated with farnesylation and/or geranylgeranylation of proteins, comprising the step of administering to the living animal body an effective amount of a compound of claim 23 for alleviation of the condition.

38. A method for treating a living animal body afflicted with a condition selected from cancers such as cancer of the lung, of the pancreas, of the skin, of the head, of the neck, of the uterus, of the ovaries, anal cancer, cancer of the stomach, of the colon, of the breast, of the esophagus, of the small intestine, of the thyroid gland, of the prostate, of the kidney, of the bladder, acute or chronic leukemia, or a combination of two or more of these cancers, comprising the step of administering to the living animal body an effective amount of a compound of claim 23 for alleviation of the condition.

39. A pharmaceutical composition of claim 36 in combination especially with an anticancer agent such as cytotoxic anticancer agents, especially navelbine, vinflunine, taxol, taxotere, 5-fluorouracil, le methotrexate, doxorubicin, camptothecin, gemcitabine, etoposide, cisplatin or BCNU, or hormonal anticancer agents, for instance tamoxifen or medroxyprogesterone, for their simultaneous, separate or sequential use, for the treatment or prevention of cancers.

40. A pharmaceutical composition of claim 36, in combination with an agent for inhibiting the biosynthesis of farnesyl and geranylgeranyl pyrophosphates, such as an HMG-CoA reductase inhibitor, for instance lovastatin, simvastatin, pravastatin, fluvastatin, atorvastatin or cerivastatin, for their simultaneous, separate or sequential use.

41. A pharmaceutical composition of claim 36, in combination with a treatment with radiation (X-rays or gamma rays) for their simultaneous, separate or sequential use for treating or preventing cancers.

42. A method of preventing or treating restenosis or atherosclerosis in a living animal body, comprising the step of administering to the living animal body a compound of claim 23 which is effective for preventing and treating such affliction.

43. A method of preventing or treating infections associated with PFTase such as delta hepatitis in a living animal body, comprising the step of administering to the living animal body a compound of claim 23 which is effective for preventing and treating such affliction.

44. A method of preventing or treating benign proliferative disorders in a living animal body, comprising the step of administering to the living animal body a compound of claim 23 which is effective for preventing and treating such affliction.
